(19) United States
(12) Patent Application Publication
(10) Pub. No.: US 2013/0303497 A1
(43) Pub. Date: Nov. 14, 2013

(54) DEUTERATED 5-HT1A RECEPTOR AGONISTS

(71) Applicants: John Bondo Hansen, Jyderup (DK); Mikael Søndergaard Thomsen, Hvidovre (DK)

(72) Inventors: John Bondo Hansen, Jyderup (DK); Mikael Søndergaard Thomsen, Hvidovre (DK)

(73) Assignee: Conrig Pharma ApS, Jyderup (DK)

(21) Appl. No.: 13/814,391

(22) PCT Filed: Aug. 4, 2011
(86) PCT No.: PCT/DK2011/050302

§ 371 Date: May 23, 2013

(30) Foreign Application Priority Data
Aug. 5, 2010 (DK) PA201070349

(51) Int. Cl. C07D 403/12; A61K 45/06; A61K 31/506

(52) U.S. Cl. CPC C07D 403/12; A61K 31/506; A61K 45/06
USPC 514/171; 544/295; 514/252.19; 514/221

(57) ABSTRACT

The present invention relates to new deuterated derivatives of serotonin 5-HT1A receptor agonists of formula 1 and in particular to compositions and methods for therapeutic use.

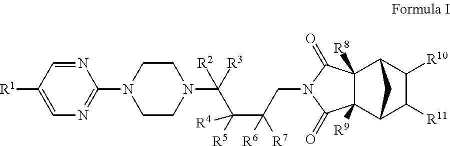

All patent and non-patent references cited in the application, or in the present application, are also hereby incorporated by reference in their entirety.

### FIELD OF INVENTION

The present invention relates to new deuterated derivatives of serotonin 5-HT1A receptor agonists and in particular to compositions and methods for therapeutic use.

### BACKGROUND OF INVENTION

Tandospirone ((1R,2R,6S,7S)-4-{4-[4-(pyrimidin-2-yl)piperazin-1-yl]butyl}-4-azatricyclo[5.2.1.0^{2,6}]decane-3,5-dione) is a member of the azapirone and piperazine chemical classes.

Tandospirone acts as a potent and selective serotonin 5-HT1A receptor partial agonist, with a Ki affinity value of 27±5 nM (Hamik et al. 1990) and approximately 55-85% intrinsic activity (Tanaka et al. 1995 and Yabuuchi et al. 2004). However, there is evidence of tandospirone having significant antagonistic activity at the α2-adrenergic receptor through its active metabolite 1-(2-pyrimidinyl)piperazine (1-PP) (Blier et al. 1991 and Miller et al. 1992).

Tandospirone and tandospirone salts have been described in several patents and patent applications. These describe pharmaceutical compositions of tandospirone alone and in combination with other drugs for treatment of human disease and include EP 0437026 (Treatment of depression), WO 1994016699 (Compositions containing tandospirone or its analogues), EP 0082402 (Succinimide derivates and process for preparation thereof), JP 2002020291 (Therapeutic agents for cognition disorders), JP 2003335678 (Therapeutic agents for neurogenic pain), WO 2004002487 (Methods for treating attention deficit disorder), JP 2005225844 (Agents for the treatment of irritable bowel syndrome), WO 2005117886 (Adhesive patch), WO 2008044336 (Crystal-containing adhesive preparation) and WO 2010065730 (Pharmaceutical suspension).

Metabolism of tandospirone is primarily mediated by CYP3A4 and to a lesser extent CYP2D6. Whereas hydroxylation of the pyrimidine ring is the major metabolite formed with CYP2D6 (M1), hydroxylation of the azatricyclo[5.2.1.0^{2,6}]decane-3,5-dione ring (M2) and 1-PP (oxidative cleavage of the butyl chain) predominates upon incubation with CYP3A4. The metabolite profile on incubation of tandospirone with CYP3A4 was qualitatively and quantitatively similar to that obtained with human liver microsomes suggesting that these metabolites are formed upon administration to humans (Natsui et al. 2007).

In humans, tandospirone has a high clearance rate, leading to a short elimination half-life in the systemic circulation around 1-2 h (Nakashima and Kanemaru 1992). In China, tandospirone (Sediel®) is therefore typically dosed 10-20 mg three times daily to maintain therapeutically relevant plasma exposure for the treatment of anxiety disorders (Lin 2011).

To fully benefit from the pharmacological profile offered by tandospirone, there is a need to improve the pharmacokinetic properties or the stability of the drug.

### SUMMARY OF THE INVENTION

The present invention provides compounds and pharmaceutical compositions comprising new tandospirone analogues wherein one or more protons are substituted with deuterium.

The inventors have surprisingly found that deuterated tandospirone, wherein one or more protons in specific positions are substituted with deuterium have properties compared to tandospirone.

The present invention provides compounds and pharmaceutical compositions comprising compounds according to Formula I:

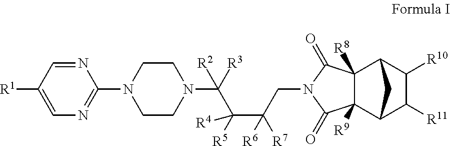

wherein R1, R2, R3, R4, R5, R6, R7, R8, R9, R10, and R11 are individually selected from the group consisting of hydrogen (H) and deuterium (D), with the proviso that at least one of R1, R2, R3, R4, R5, R6, R7, R8, R9, R10, and R11 is deuterium.

In one embodiment of the present invention, the compounds as defined herein have increased stability and/or altered pharmacokinetic profile compared to the compound of formula I wherein all of R1, R2, R3, R4, R5, R6, R7, R8, R9, R10 and R11 are hydrogen (tandospirone). For example, in such an embodiment, the rate of intrinsic clearance of the deuterated compound as defined herein measured by incubation with human liver microsomes can be increased compared to non-deuterated tandospirone, for example such as increased to a range of 1 ml/min/kg to 540 ml/min/kg. In another embodiment, altered pharmacokinetic profile is indicated by a reduced plasma protein binding of the deuterated compound as defined herein compared to tandospirone, such as a plasma protein binding in the range of 1-99% compared to tandospirone, preferably 1-83% compared to tandospirone. In other embodiments altered pharmacokinetic profile of the deuterated compounds as defined herein is indicated by increased apparent permeability through a biomembrane, and/or decreased inhibition of CYP34A mediated metabolism compared to tandospirone.

In preferred embodiments of the present invention compounds according to formula I have deuterium in one or both of the positions R2 and R3 and the positions R1, R4, R5, R6, R7, R8, R9, R10 and R11 are selected from deuterium or hydrogen,

In an even more preferred embodiment of the present invention, the compounds according to formula I are selected from the group of (1R,2R,6S,7S)-4-{4-[4-(pyrimidin-2-yl)piperazin-1-yl]-(4,4-²H₂)butyl}-4-azatricyclo-[5.2.1.0^{2,6}]decane-3,5-dione (II), (1R,2R,6S,7S)-4-{4-[4-((5-²H)pyrimidin-2-yl)piperazin-1-yl]-(4,4-²H₂)butyl}-4-azatricyclo[5.2.1.0^{2,6}]decane-3,5-dione (III), (1R,2R,6S,7S)-4-{4-[4-(pyrimidin-2-yl)piperazin-1-yl]-(4,4-²H₂)butyl}-(8,9-²H₂)-4-azatricyclo[5.2.1.0^{2,6}]decane-3,5-dione (VI), (1R,2R,6S,7S)-4-{4-[4-(((5-²H)pyrimidin-2-yl)piperazin-1-yl]-(4,4-²H₂)butyl}-(2,6-²H₂)-4-azatricyclo[5.2.1.0^{2,6}]decane-3,5-dione (VII), (1R,2R,6S,7S)-4-{4-[4-(pyrimidin-2-yl)piperazin-1-yl]-(4-²H₂)butyl}-(2,6-²H₂)-4-azatricyclo[5.2.1.0^{2,6}]decane-3,5-dione XV, (1R,2R,6S,7S)-4-{4-[4-(pyrimidin-2-yl)piperazin-1-yl]-(2,2,3,3,4,4-²H₆)butyl}-4-azatricyclo[5.2.1.0^{2,6}]decane-3,5-dione (XVIII).

The present invention further provides pharmaceutical composition comprising a compound defined in any of the preceding claims, wherein deuterium is incorporated in one or more of R1, R2, R3, R4, R5, R6, R7, R8, R9, R10, and R11 in at least 50% of the compounds, such as in at least 55% of the compounds, such as at least 60% of the compounds, such as at least 65% of the compounds, such as at least 70% of the compounds, such as at least 75% of the compounds, such as at least 80% of the compounds, such as at least 85% of the compounds, such as at least 90% of the compounds, such as at least 95% of the compounds, such as at least 96% of the compounds, such as at least 97% of the compounds, such as at least 98% of the compounds, such as at least 99% of the compounds, such as at least 99.5% of the compounds, such as at least 99.9% of the compounds, or pharmaceutically acceptable salts, acid addition salts or base addition salts thereof and a pharmaceutically acceptable carrier.

The compounds, pharmaceutical compositions and methods according the present invention are useful for treatment of diseases or conditions where activation of the serotonin 5-HT1A receptor will have a beneficial therapeutic effect, or for diseases associated with dysfunction of the serotonin 5-HT1A receptor.

In one particular embodiment, the present invention to provides compounds, pharmaceutical compositions and methods for treatment of dermatological disorders selected from the group of atopic dermatitis, seborrhoeic dermatitis, diaper dermatitis, allergic contact dermatitis, irritant contact dermatitis, unspecified contact dermatitis, infective dermatitis, exfoliative dermatitis, lichen simplex chronicus, lichen planus, pruritus/itch, pityriasis rosea, rosacea, psoriasis, urticaria (allergic and unspecified), erythema, sunburn, pemphigus and other acantholytic disorders, dermatological disorders associated with stress and treatment of dermatological disorders associated with diseases of the central nervous system such as anxiety and depressions, dermatological disorders associated with stress, and dermatological disorders associated with diseases of the central nervous system such as anxiety and depressions.

In another embodiment of the present invention, the compounds as defined herein are used for treatment of disorders of the central nervous system, cognitive impairment/dysfunction disorders, eating disorders, dyspepsia, treatment of development of tolerance to the treatment effects of morphine, opiates and alcohol, treatment of dependency of alcohol or tobacco smoking, treatment of dyspepsia, acute, chronic or idiopathic cough, age related macular degeneration (AMD) and sexual dysfunction, or impairments, and or dysfunctions caused by cerebral ischemia, or movement disorders.

In another embodiment of the present invention, the compounds or pharmaceutical compositions as defined herein are used for treatment of acute pain, chronic pain, visceral pain, neuropathic pain.

In another embodiment of the present invention, the compounds or pharmaceutical compositions as defined herein are used for treatment or prevention of postoperative nausea and vomiting (PONV), cancer-induced nausea and vomiting (CINV).

The pharmaceutical compositions of the present invention may further comprise one or more second active ingredients.

In one embodiment of the present invention, the second active ingredient is selected from the group of serotonin reuptake inhibitors, corticosteroids, antihistamines, immunomodulators, vitamin derivatives, biologics and NK-1 antagonists.

In another embodiment of the present invention, the second active ingredient is selected from analgesic medication classes including NSAIDs, COX-2 inhibitors, acetaminophen, other anti-inflammatory, tricyclic antidepressants, anticonvulsant agents, voltage gated calcium channel blockers, N-type calcium channel blockers, other calcium channel modulators, SNRI and other monoamine reuptake inhibitors, sodium channel blockers, NMDA antagonists, AMPA antagonists, other glutamate modulators, GABA modulators, CRMP-2 modulators, NK-1 antagonists, TRPV1 agonists, cannabinoids, adenosine agonists, nicotinic agonists, p38 MAP kinase inhibitors, corticosteroids, triptans used for treatment and prevention of migraine, strong and weak opioids selected from fentanyl, oxycodone, codeine, dihydrocodeine, hydrocodone, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papvereturn, alfentanil, buprenorphine and tramadol and other analgesic drug classes, wherein preferred opioids are selected from the group of hydrocodone, oxycodone, codeine or tramadol.

In yet another embodiment of the present invention, the pharmaceutical composition comprises a second active ingredient selected from antiemetic agents including 5-HT3 antagonists, NK-1 antagonists, dopamine antagonists, H1 histamine receptor antagonists, cannabinoids, benzodiazepines, anticholinergic compounds and steroid compounds.

The pharmaceutical compositions according to the present invention are suitable for oral, rectal, nasal, pulmonary, buccal, sublingual, transdermal and parenteral administration.

In a preferred embodiment of the present invention, compounds and pharmacological compositions of the present invention are administered orally.

The pharmaceutical compositions according to the present invention for allow for administering the compounds as defined by formula I in a therapeutically effective amount, such as doses of 0.001 to 1000 mg, such as 0.01 to 600 mg, or such as 0.5 mg to 200 mg.

The present invention further provides a kit of parts comprising the pharmaceutical compositions as defined by the present invention for simultaneous, sequential or separate administration which may comprise a second active ingredient as defined by the present invention.

The methods for treatment of diseases or disorders according to the present invention comprise separate, sequential or/and simultaneous administration of a therapeutically effective amount of a pharmaceutical compositions according to the present invention to an individual in need thereof.

Further, the present invention provides methods for synthesis of deuterated compounds according to formula I:

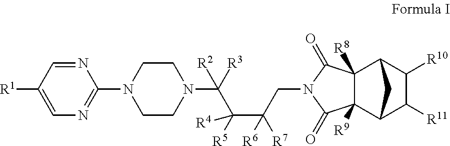

wherein R1, R2, R3, R10 and R11 are selected from the group consisting of hydrogen (H) and deuterium (D) with the proviso that at least one of R1, R2, R3, R10 and R11 is deuterium.

### DEFINITIONS

### Compound

The term “compound” as used herein, refers to a collection of molecules having an identical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure.

### Impure Isotopologue

The term “impure isotopologue” refers to a species that differs from specific compounds of this invention only in the isotopic composition thereof. It will be recognized that some variations of the natural isotopic abundance occurs in a synthesized compound depending upon the origin of chemical materials used in the synthesis. Thus, a preparation of deuterated compound according to formula I will inherently contain small amounts of impure isotopologues.

### Isotopic Enrichment Factor

The term “isotopic enrichment factor” as used herein means the ratio between the isotopic abundance and the natural abundance of a specific isotope. When a position is designated specifically as “D” or “deuterium”, the position is understood to have deuterium at an abundance that is substantially greater than natural abundance of deuterium which is 0.015%. All percentages given for the amount of deuterium present are mole percentages. It is thus understood that pharmaceutical compositions according to the present invention comprise compounds which have isotopic enrichment factors significantly above 1.

### Isotopologue

The term “isotopologue” refers to a species that differs from specific compounds of this invention only in the isotopic composition thereof.

### Pharmaceutical Composition

The term “pharmaceutical composition” as used herein, refers to compositions comprising compounds according to formula I, which have identical structure, except that there may be isotopic variation among the constituent atoms of the molecules. Thus, it will be clear to those of skill in the art that a compound represented by a particular chemical structure containing indicated deuterium atoms, will also contain lesser amounts of impure isotopologues having hydrogen atoms at one or more of the designated deuterium positions in that structure.

### Pharmaceutically Acceptable Salt

In the present context, the term “pharmaceutically acceptable salt” is intended to indicate a salt which is not harmful to the patient. Such salts include pharmaceutically acceptable basic or acid addition salts as well as pharmaceutically acceptable metal salts, ammonium salts and alkylated ammonium salts.

### Prodrug

As used herein, the term “prodrug” includes derivatives of compounds of the invention such as biohydrolyzable amides and biohydrolyzable esters thereof, or compounds defined as follows:
a) compounds in which the biohydrolyzable functionality in such a prodrug is encompassed in the compound according to the present invention; and
b) compounds which may be oxidized or reduced biologically at a given functional group to yield drug substances according to the present invention.

Examples of the latter type of functional group include 1,4-dihydropyridine, N-alkylcarbonyl-1,4-dihydropyridine, 1,4-cyclohexadiene, tert-butyl and the like.

### Solvate

As used herein, the term “solvate” refers to a complex of defined stoichiometry formed by a solute (in casu, a compound according to the present invention) and a solvent. Solvents according to the present invention include, by way of example, water, ethanol and acetic acid.

### Therapeutically Effective Amount

The term “therapeutically effective amount” of a compound as used herein refers to an amount sufficient to cure, alleviate or partially arrest the clinical manifestations of a given disease or disorder and its complications. An amount adequate to accomplish this is defined as a “therapeutically effective amount”.

### Treatment

The terms “treatment” and “treating” as used herein refer to the management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” is to be understood to refer to the management and care of a patient for the purpose of hindering or decreasing the risk of the development of the condition, disease or disorder, and includes the administration of the active compounds to prevent the onset of symptoms or complications. The patient to be treated is preferably a mammal, in particular a human being. Treatment of animals, such as dogs, cats, cows, sheep and pigs, is, however, also within the scope of the present invention. The patients to be treated according to the present invention are of various ages.

### Tandospirone

The compound tandospirone as mentioned herein denotes non-deuterated tandospirone, thus a compound according to Formula I wherein all positions R1, R2, R3, R4, R5, R6, R7, R8, R9, R10 and R11 are hydrogen. A preparation of tandospirone (non-deuterated tandospirone) as mentioned herein may comprise compounds wherein deuterium is incorporated in abundance in the range of the natural abundance of deuterium or where the isotopic enrichment factor is close to or equal to 1.

### DETAILED DESCRIPTION OF THE INVENTION

The current invention relates to new analogues of tandospirone, to methods of synthesis and to methods for therapeutic use. In the new tandospirone analogues, one or more protons are substituted with deuterium.

The inventors have surprisingly found that tandospirone analogues wherein one or more protons are substituted with deuterium in specific positions have altered properties compared to tandospirone.

The new analogues are compounds of the Formula I:

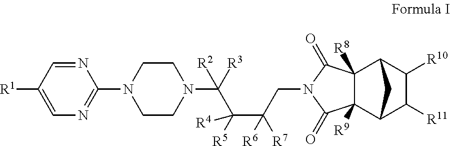

or pharmaceutical acceptable salts thereof,
wherein R1, R2, R3, R4, R5, R6, R7, R8, R9, R10, and R11 are individually selected from the group consisting of hydrogen (H) and deuterium (D), and with the proviso that at least one of R1, R2, R3, R4, R5, R6, R7, R8, R9, R10, and R11 is deuterium.

The new analogues of tandospirone may thus be isotopic labeled with deuterium in one or more of the positions selected from R1, R2, R3, R4, R5, R6, R7, R8, R9, R10, and R11 according to formula I.

Thus, according to the present invention, the new analogues of tandospirone may be labeled with deuterium in one or more of the positions as indicated in Table I below, wherein R1, R2, R3, R4, R5, R6, R7, R8, R9, R10 and R11 indicate positions in formula I, “+” denotes that the corresponding positions is a deuterium, and space (the absence of “+”) denotes that the corresponding positions are hydrogen:

TABLE I

| R1 | R2 | R3 | R4 | R5 | R6 | R7 | R8 | R9 | R1 | R1 |
|---|---|---|---|---|---|---|---|---|---|---|
| + | | | | | | | | | | |
| | + | | | | | | | | | |
| | | + | | | | | | | | |
| | | | + | | | | | | | |
| | | | | + | | | | | | |
| | | | | | + | | | | | |
| | | | | | | + | | | | |
| | | | | | | | + | | | |
| | | | | | | | | + | | |
| | | | | | | | | | + | |
| | | | | | | | | | | + |
| + | + | | | | | | | | | |
| + | | + | | | | | | | | |
| + | | | + | | | | | | | |
| + | | | | + | | | | | | |
| + | | | | | + | | | | | |
| + | | | | | | + | | | | |
| + | | | | | | | + | | | |
| + | | | | | | | | + | | |
| + | | | | | | | | | + | |
| + | | | | | | | | | | + |
| | + | + | | | | | | | | |
| | + | | + | | | | | | | |
| | + | | | + | | | | | | |
| | + | | | | + | | | | | |
| | + | | | | | + | | | | |
| | + | | | | | | + | | | |
| | + | | | | | | | + | | |
| | + | | | | | | | | + | |
| | + | | | | | | | | | + |
| | | + | + | | | | | | | |
| | | + | | + | | | | | | |
| | | + | | | + | | | | | |
| | | + | | | | + | | | | |
| | | + | | | | | + | | | |
| | | + | | | | | | + | | |
| | | + | | | | | | | + | |
| | | + | | | | | | | | + |
| | | | + | + | | | | | | |
| | | | + | | + | | | | | |
| | | | + | | | + | | | | |
| | | | + | | | | + | | | |
| | | | + | | | | | + | | |
| | | | + | | | | | | + | |
| | | | + | | | | | | | + |
| | | | | + | + | | | | | |
| | | | | + | | + | | | | |
| | | | | + | | | + | | | |
| | | | | + | | | | + | | |
| | | | | + | | | | | + | |
| | | | | + | | | | | | + |
| | | | | | + | + | | | | |
| | | | | | + | | + | | | |
| | | | | | + | | | + | | |
| | | | | | + | | | | + | |
| | | | | | + | | | | | + |
| | | | | | | + | + | | | |
| | | | | | | + | | + | | |
| | | | | | | + | | | + | |
| | | | | | | + | | | | + |
| | | | | | | | + | + | | |
| | | | | | | | + | | + | |
| | | | | | | | + | | | + |
| | | | | | | | | + | + | |
| | | | | | | | | + | | + |
| | | | | | | | | | + | + |
| + | + | + | | | | | | | | |
| + | + | | + | | | | | | | |
| + | + | | | + | | | | | | |
| + | + | | | | + | | | | | |
| + | + | | | | | + | | | | |
| + | + | | | | | | + | | | |
| + | + | | | | | | | + | | |
| + | + | | | | | | | | + | |
| + | | + | + | | | | | | | |
| + | | + | | + | | | | | | |
| + | | + | | | + | | | | | |
| + | | + | | | | + | | | | |
| + | | + | | | | | + | | | |
| + | | + | | | | | | + | | |
| + | | + | | | | | | | + | |
| + | | + | | | | | | | | + |
| + | | | + | + | | | | | | |
| + | | | + | | + | | | | | |
| + | | | + | | | + | | | | |
| + | | | + | | | | + | | | |
| + | | | + | | | | | + | | |
| + | | | + | | | | | | + | |
| + | | | + | | | | | | | + |
| + | | | | + | + | | | | | |
| + | | | | + | | + | | | | |
| + | | | | + | | | + | | | |
| + | | | | + | | | | + | | |
| + | | | | + | | | | | + | |
| + | | | | + | | | | | | + |
| + | | | | | + | + | | | | |
| + | | | | | + | | + | | | |
| + | | | | | + | | | + | | |
| + | | | | | + | | | | + | |
| + | | | | | + | | | | | + |
| + | | | | | | + | + | | | |
| + | | | | | | + | | + | | |
| + | | | | | | + | | | + | |
| + | | | | | | + | | | | + |
| + | | | | | | | + | + | | |
| + | | | | | | | + | | + | |
| + | | | | | | | + | | | + |
| + | | | | | | | | + | + | |
| + | | | | | | | | + | | + |
| | + | + | + | | | | | | | |
| | + | + | | + | | | | | | |
| | + | + | | | + | | | | | |
| | + | + | | | | + | | | | |
| | + | + | | | | | + | | | |
| | + | + | | | | | | + | | |
| | + | + | | | | | | | + | |
| | + | + | | | | | | | | + |
| | + | | + | + | | | | | | |
| | + | | + | | + | | | | | |
| | + | | + | | | + | | | | |
| | + | | + | | | | + | | | |
| | + | | + | | | | | + | | |
| | + | | + | | | | | | + | |
| | + | | + | | | | | | | + |
| | + | | | + | + | | | | | |
| | + | | | + | | + | | | | |
| | + | | | + | | | + | | | |
| | + | | | + | | | | + | | |
| | + | | | + | | | | | + | |
| | + | | | + | | | | | | + |
| | + | | | | + | + | | | | |
| | + | | | | + | | + | | | |
| | + | | | | + | | | + | | |
| | + | | | | + | | | | + | |
| | + | | | | + | | | | | + |
| | + | | | | | + | + | | | |
| | + | | | | | + | | + | | |
| | + | | | | | + | | | + | |
| | + | | | | | + | | | | + |
| | + | | | | | | + | + | | |
| | + | | | | | | + | | + | |
| | + | | | | | | + | | | + |
| | + | | | | | | | + | + | |
| | + | | | | | | | + | | + |
| | + | | | | | | | | + | + |
| | | + | + | + | | | | | | |
| | | + | + | | + | | | | | |
| | | + | + | | | + | | | | |
| | | + | + | | | | + | | | |
| | | + | + | | | | | + | | |
| | | + | + | | | | | | + | |
| | | + | + | | | | | | | + |
| | | + | | + | + | | | | | |
| | | + | | + | | + | | | | |
| | | + | | + | | | + | | | |
| | | + | | + | | | | + | | |
| | | + | | + | | | | | + | |
| | | + | | + | | | | | | + |
| | | + | | | + | + | | | | |
| | | + | | | + | | + | | | |
| | | + | | | + | | | + | | |
| | | + | | | + | | | | + | |
| | | + | | | + | | | | | + |
| | | + | | | | + | + | | | |
| | | + | | | | + | | + | | |
| | | + | | | | + | | | + | |
| | | + | | | | + | | | | + |
| | | + | | | | | + | + | | |
| | | + | | | | | + | | + | |
| | | + | | | | | + | | | + |
| | | + | | | | | | + | + | |
| | | + | | | | | | + | | + |
| | | + | | | | | | | + | + |
| | | | + | + | + | | | | | |
| | | | + | + | | + | | | | |
| | | | + | + | | | + | | | |
| | | | + | + | | | | + | | |
| | | | + | + | | | | | + | |
| | | | + | + | | | | | | + |
| | | | + | | + | + | | | | |
| | | | + | | + | | + | | | |
| | | | + | | + | | | + | | |
| | | | + | | + | | | | + | |
| | | | + | | + | | | | | + |
| | | | + | | | + | + | | | |
| | | | + | | | + | | + | | |
| | | | + | | | + | | | + | |
| | | | + | | | + | | | | + |
| | | | + | | | | + | + | | |
| | | | + | | | | + | | + | |
| | | | + | | | | + | | | + |
| | | | + | | | | | + | + | |
| | | | + | | | | | + | | + |
| | | | + | | | | | | + | + |
| | | | | + | + | + | | | | |
| | | | | + | + | | + | | | |
| | | | | + | + | | | + | | |
| | | | | + | + | | | | + | |
| | | | | + | + | | | | | + |
| | | | | + | | + | + | | | |
| | | | | + | | + | | + | | |
| | | | | + | | + | | | + | |
| | | | | + | | + | | | | + |
| | | | | + | | | + | + | | |
| | | | | + | | | + | | + | |
| | | | | + | | | + | | | + |
| | | | | + | | | | + | + | |
| | | | | + | | | | + | | + |
| | | | | + | | | | | + | + |
| | | | | | + | + | + | | | |
| | | | | | + | + | | + | | |
| | | | | | + | + | | | + | |
| | | | | | + | + | | | | + |
| | | | | | + | | + | + | | |
| | | | | | + | | + | | + | |
| | | | | | + | | + | | | + |
| | | | | | + | | | + | + | |
| | | | | | + | | | + | | + |
| | | | | | + | | | | + | + |
| | | | | | | + | + | + | | |
| | | | | | | + | + | | + | |
| | | | | | | + | + | | | + |
| | | | | | | + | | + | + | |
| | | | | | | + | | + | | + |
| | | | | | | + | | | + | + |
| | | | | | | | + | + | + | |
| | | | | | | | + | + | | + |
| | | | | | | | + | | + | + |
| | | | | | | | | + | + | + |
| + | + | + | + | | | | | | | |
| + | + | + | | + | | | | | | |
| + | + | + | | | + | | | | | |
| + | + | + | | | | + | | | | |
| + | + | + | | | | | + | | | |
| + | + | + | | | | | | + | | |
| + | + | + | | | | | | | + | |
| + | + | + | | | | | | | | + |
| + | + | | + | + | | | | | | |
| + | + | | + | | + | | | | | |
| + | + | | + | | | + | | | | |
| + | + | | + | | | | + | | | |
| + | + | | + | | | | | + | | |
| + | + | | + | | | | | | + | |
| + | + | | + | | | | | | | + |
| + | + | | | + | + | | | | | |
| + | + | | | + | | + | | | | |
| + | + | | | + | | | + | | | |
| + | + | | | + | | | | + | | |
| + | + | | | + | | | | | + | |
| + | + | | | + | | | | | | + |
| + | + | | | | + | + | | | | |
| + | + | | | | + | | + | | | |
| + | + | | | | + | | | + | | |
| + | + | | | | + | | | | + | |
| + | + | | | | + | | | | | + |
| + | + | | | | | + | + | | | |
| + | + | | | | | + | | + | | |
| + | + | | | | | + | | | + | |
| + | + | | | | | + | | | | + |
| + | + | | | | | | + | + | | |
| + | + | | | | | | + | | + | |
| + | + | | | | | | + | | | + |
| + | + | | | | | | | + | + | |
| + | + | | | | | | | + | | + |
| + | + | | | | | | | | + | + |
| + | | + | + | + | | | | | | |
| + | | + | + | | + | | | | | |
| + | | + | + | | | + | | | | |
| + | | + | + | | | | + | | | |
| + | | + | + | | | | | + | | |
| + | | + | + | | | | | | + | |
| + | | + | + | | | | | | | + |
| + | | + | | + | + | | | | | |
| + | | + | | + | | + | | | | |
| + | | + | | + | | | + | | | |
| + | | + | | + | | | | + | | |
| + | | + | | + | | | | | + | |
| + | | + | | + | | | | | | + |
| + | | + | | | + | + | | | | |
| + | | + | | | + | | + | | | |
| + | | + | | | + | | | + | | |
| + | | + | | | + | | | | + | |
| + | | + | | | + | | | | | + |
| + | | + | | | | + | + | | | |
| + | | + | | | | + | | + | | |
| + | | + | | | | + | | | + | |
| + | | + | | | | + | | | | + |
| + | | + | | | | | + | + | | |
| + | | + | | | | | + | | + | |
| + | | + | | | | | + | | | + |
| + | | + | | | | | | + | + | |
| + | | + | | | | | | + | | + |
| + | | + | | | | | | | + | + |
| + | | | + | + | + | | | | | |
| + | | | + | + | | + | | | | |
| + | | | + | + | | | + | | | |
| + | | | + | + | | | | + | | |
| + | | | + | + | | | | | + | |
| + | | | + | + | | | | | | + |
| + | | | + | | + | + | | | | |
| + | | | + | | + | | + | | | |
| + | | | + | | + | | | + | | |
| + | | | + | | + | | | | + | |
| + | | | + | | + | | | | | + |
| + | | | + | | | + | + | | | |
| + | | | + | | | + | | + | | |
| + | | | + | | | + | | | + | |
| + | | | + | | | + | | | | + |
| + | | | + | | | | + | + | | |
| + | | | + | | | | + | | + | |
| + | | | + | | | | + | | | + |
| + | | | + | | | | | + | + | |
| + | | | + | | | | | + | | + |
| + | | | + | | | | | | + | + |
| + | | | | + | + | + | | | | |
| + | | | | + | + | | + | | | |
| + | | | | + | + | | | + | | |
| + | | | | + | + | | | | + | |
| + | | | | + | + | | | | | + |
| + | | | | + | | + | + | | | |
| + | | | | + | | + | | + | | |
| + | | | | + | | + | | | + | |
| + | | | | + | | + | | | | + |
| + | | | | + | | | + | + | | |
| + | | | | + | | | + | | + | |
| + | | | | + | | | + | | | + |
| + | | | | + | | | | + | + | |
| + | | | | + | | | | + | | + |
| + | | | | + | | | | | + | + |
| + | | | | | + | + | + | | | |
| + | | | | | + | + | | + | | |
| + | | | | | + | + | | | + | |
| + | | | | | + | + | | | | + |
| + | | | | | + | | + | + | | |
| + | | | | | + | | + | | + | |
| + | | | | | + | | + | | | + |
| + | | | | | + | | | + | + | |
| + | | | | | + | | | + | | + |
| + | | | | | + | | | | + | + |
| + | | | | | | + | + | + | | |
| + | | | | | | + | + | | + | |
| + | | | | | | + | + | | | + |
| + | | | | | | + | | + | + | |
| + | | | | | | + | | + | | + |
| + | | | | | | | + | + | + | |
| + | | | | | | | + | + | | + |
| + | | | | | | | + | | + | + |
| + | | | | | | | | + | + | + |
| | + | + | + | + | | | | | | |
| | + | + | + | | + | | | | | |
| | + | + | + | | | + | | | | |
| | + | + | + | | | | + | | | |
| | + | + | + | | | | | + | | |
| | + | + | + | | | | | | + | |
| | + | + | + | | | | | | | + |
| | + | + | | + | + | | | | | |
| | + | + | | + | | + | | | | |
| | + | + | | + | | | + | | | |
| | + | + | | + | | | | + | | |
| | + | + | | + | | | | | + | |
| | + | + | | + | | | | | | + |
| | + | + | | | + | + | | | | |
| | + | + | | | + | | + | | | |
| | + | + | | | + | | | + | | |
| | + | + | | | + | | | | + | |
| | + | + | | | + | | | | | + |
| | + | + | | | | + | + | | | |
| | + | + | | | | + | | + | | |
| | + | + | | | | + | | | + | |
| | + | + | | | | + | | | | + |
| | + | + | | | | | + | + | | |
| | + | + | | | | | + | | + | |
| | + | + | | | | | + | | | + |
| | + | + | | | | | | + | + | |
| | + | + | | | | | | + | | + |
| | + | + | | | | | | | + | + |
| | + | | + | + | + | | | | | |
| | + | | + | + | | + | | | | |
| | + | | + | + | | | + | | | |
| | + | | + | + | | | | + | | |
| | + | | + | + | | | | | + | |
| | + | | + | + | | | | | | + |
| | + | | + | | + | + | | | | |
| | + | | + | | + | | + | | | |
| | + | | + | | + | | | + | | |
| | + | | + | | + | | | | + | |
| | + | | + | | + | | | | | + |
| | + | | + | | | + | + | | | |
| | + | | + | | | + | | + | | |
| | + | | + | | | + | | | + | |
| | + | | + | | | + | | | | + |
| | + | | + | | | | + | + | | |
| | + | | + | | | | + | | + | |
| | + | | + | | | | + | | | + |
| | + | | + | | | | | + | + | |
| | + | | + | | | | | + | | + |
| | + | | + | | | | | | + | + |
| | + | | | + | + | + | | | | |
| | + | | | + | + | | + | | | |
| | + | | | + | + | | | + | | |
| | + | | | + | + | | | | + | |
| | + | | | + | + | | | | | + |
| | + | | | + | | + | + | | | |
| | + | | | + | | + | | + | | |
| | + | | | + | | + | | | + | |
| | + | | | + | | + | | | | + |
| | + | | | + | | | + | + | | |
| | + | | | + | | | + | | + | |
| | + | | | + | | | + | | | + |
| | + | | | + | | | | + | + | |
| | + | | | + | | | | + | | + |
| | + | | | + | | | | | + | + |
| | + | | | | + | + | + | | | |
| | + | | | | + | + | | + | | |
| | + | | | | + | + | | | + | |
| | + | | | | + | + | | | | + |
| | + | | | | + | | + | + | | |
| | + | | | | + | | + | | + | |
| | + | | | | + | | + | | | + |
| | + | | | | + | | | + | + | |
| | + | | | | + | | | + | | + |
| | + | | | | + | | | | + | + |
| | + | | | | | + | + | + | | |
| | + | | | | | + | + | | + | |
| | + | | | | | + | + | | | + |
| | + | | | | | + | | + | + | |
| | + | | | | | + | | + | | + |
| | + | | | | | + | | | + | + |
| | + | | | | | | + | + | + | |
| | + | | | | | | + | + | | + |
| | + | | | | | | + | | + | + |
| | + | | | | | | | + | + | + |
| | | + | + | + | + | | | | | |
| | | + | + | + | | + | | | | |
| | | + | + | + | | | + | | | |
| | | + | + | + | | | | + | | |
| | | + | + | + | | | | | + | |
| | | + | + | + | | | | | | + |
| | | + | + | | + | + | | | | |
| | | + | + | | + | | + | | | |
| | | + | + | | + | | | + | | |
| | | + | + | | + | | | | + | |
| | | + | + | | + | | | | | + |
| | | + | + | | | + | + | | | |
| | | + | + | | | + | | + | | |
| | | + | + | | | + | | | + | |
| | | + | + | | | + | | | | + |
| | | + | + | | | | + | + | | |
| | | + | + | | | | + | | + | |
| | | + | + | | | | + | | | + |
| | | + | + | | | | | + | + | |
| | | + | + | | | | | + | | + |
| | | + | + | | | | | | + | + |
| | | + | | + | + | + | | | | |
| | | + | | + | + | | + | | | |
| | | + | | + | + | | | + | | |
| | | + | | + | + | | | | + | |
| | | + | | + | + | | | | | + |
| | | + | | + | | + | + | | | |
| | | + | | + | | + | | + | | |
| | | + | | + | | + | | | + | |
| | | + | | + | | + | | | | + |
| | | + | | + | | | + | + | | |
| | | + | | + | | | + | | + | |
| | | + | | + | | | + | | | + |
| | | + | | + | | | | + | + | |
| | | + | | + | | | | + | | + |
| | | + | | + | | | | | + | + |
| | | + | | | + | + | + | | | |
| | | + | | | + | + | | + | | |
| | | + | | | + | + | | | + | |
| | | + | | | + | + | | | | + |
| | | + | | | + | | + | + | | |
| | | + | | | + | | + | | + | |
| | | + | | | + | | + | | | + |
| | | + | | | + | | | + | + | |
| | | + | | | + | | | + | | + |
| | | + | | | + | | | | + | + |
| | | + | | | | + | + | + | | |
| | | + | | | | + | + | | + | |
| | | + | | | | + | + | | | + |
| | | + | | | | + | | + | + | |
| | | + | | | | + | | + | | + |
| | | + | | | | + | | | + | + |
| | | + | | | | | + | + | + | |
| | | + | | | | | + | + | | + |
| | | + | | | | | + | | + | + |
| | | + | | | | | | + | + | + |
| | | | + | + | + | + | | | | |
| | | | + | + | + | | + | | | |
| | | | + | + | + | | | + | | |
| | | | + | + | + | | | | + | |
| | | | + | + | + | | | | | + |
| | | | + | + | | + | + | | | |
| | | | + | + | | + | | + | | |
| | | | + | + | | + | | | + | |
| | | | + | + | | + | | | | + |
| | | | + | + | | | + | + | | |
| | | | + | + | | | + | | + | |
| | | | + | + | | | + | | | + |
| | | | + | + | | | | + | + | |
| | | | + | + | | | | + | | + |
| | | | + | + | | | | | + | + |
| | | | + | | + | + | + | | | |
| | | | + | | + | + | | + | | |
| | | | + | | + | + | | | + | |
| | | | + | | + | + | | | | + |
| | | | + | | + | | + | + | | |
| | | | + | | + | | + | | + | |
| | | | + | | + | | + | | | + |
| | | | + | | + | | | + | + | |
| | | | + | | + | | | + | | + |
| | | | + | | + | | | | + | + |
| | | | + | | | + | + | + | | |
| | | | + | | | + | + | | + | |
| | | | + | | | + | + | | | + |
| | | | + | | | + | | + | + | |
| | | | + | | | + | | + | | + |
| | | | + | | | + | | | + | + |
| | | | + | | | | + | + | + | |
| | | | + | | | | + | + | | + |
| | | | + | | | | + | | + | + |
| | | | + | | | | | + | + | + |
| | | | | + | + | + | + | | | |
| | | | | + | + | + | | + | | |
| | | | | + | + | + | | | + | |
| | | | | + | + | + | | | | + |
| | | | | + | + | | + | + | | |
| | | | | + | + | | + | | + | |
| | | | | + | + | | + | | | + |
| | | | | + | + | | | + | + | |
| | | | | + | + | | | + | | + |
| | | | | + | + | | | | + | + |
| | | | | + | | + | + | + | | |
| | | | | + | | + | + | | + | |
| | | | | + | | + | + | | | + |
| | | | | + | | + | | + | + | |
| | | | | + | | + | | + | | + |
| | | | | + | | + | | | + | + |
| | | | | + | | | + | + | + | |
| | | | | + | | | + | + | | + |
| | | | | + | | | + | | + | + |
| | | | | + | | | | + | + | + |
| | | | | | + | + | + | + | | |
| | | | | | + | + | + | | + | |
| | | | | | + | + | + | | | + |
| | | | | | + | + | | + | + | |
| | | | | | + | + | | + | | + |
| | | | | | + | + | | | + | + |
| | | | | | + | | + | + | + | |
| | | | | | + | | + | + | | + |
| | | | | | + | | + | | + | + |
| | | | | | + | | | + | + | + |
| | | | | | | + | + | + | + | |
| | | | | | | + | + | + | | + |
| | | | | | | + | + | | + | + |
| | | | | | | + | | + | + | + |
| | | | | | | | + | + | + | + |
| + | + | + | + | + | | | | | | |
| + | + | + | + | | + | | | | | |
| + | + | + | + | | | + | | | | |
| + | + | + | + | | | | + | | | |
| + | + | + | + | | | | | + | | |
| + | + | + | + | | | | | | + | |
| + | + | + | + | | | | | | | + |
| + | + | + | | + | + | | | | | |
| + | + | + | | + | | + | | | | |
| + | + | + | | + | | | + | | | |
| + | + | + | | + | | | | + | | |
| + | + | + | | + | | | | | + | |
| + | + | + | | + | | | | | | + |
| + | + | + | | | + | + | | | | |
| + | + | + | | | + | | + | | | |
| + | + | + | | | + | | | + | | |
| + | + | + | | | + | | | | + | |
| + | + | + | | | + | | | | | + |
| + | + | + | | | | + | + | | | |
| + | + | + | | | | + | | + | | |
| + | + | + | | | | + | | | + | |
| + | + | + | | | | + | | | | + |
| + | + | + | | | | | + | + | | |
| + | + | + | | | | | + | | + | |
| + | + | + | | | | | + | | | + |
| + | + | + | | | | | | + | + | |
| + | + | + | | | | | | + | | + |
| + | + | + | | | | | | | + | + |
| + | + | | + | + | + | | | | | |
| + | + | | + | + | | + | | | | |
| + | + | | + | + | | | + | | | |
| + | + | | + | + | | | | + | | |
| + | + | | + | + | | | | | + | |
| + | + | | + | + | | | | | | + |
| + | + | | + | + | | | | | | |
| + | + | | + | | + | + | | | | |
| + | + | | + | | + | | + | | | |
| + | + | | + | | + | | | + | | |
| + | + | | + | | + | | | | + | |
| + | + | | + | | + | | | | | + |
| + | + | | + | | + | | | | | |
| + | + | | + | | | + | + | | | |
| + | + | | + | | | + | | + | | |
| + | + | | + | | | + | | | + | |
| + | + | | + | | | + | | | | + |
| + | + | | + | | | | + | + | | |
| + | + | | + | | | | + | | + | |
| + | + | | + | | | | + | | | + |
| + | + | | + | | | | | + | + | |
| + | + | | + | | | | | + | | + |
| + | + | | + | | | | | | + | + |
| + | + | | | + | + | + | | | | |
| + | + | | | + | + | | + | | | |
| + | + | | | + | + | | | + | | |
| + | + | | | + | + | | | | + | |
| + | + | | | + | + | | | | | + |
| + | + | | | + | | + | + | | | |
| + | + | | | + | | + | | + | | |
| + | + | | | + | | + | | | + | |
| + | + | | | + | | + | | | | + |
| + | + | | | + | | | + | + | | |
| + | + | | | + | | | + | | + | |
| + | + | | | + | | | + | | | + |
| + | + | | | + | | | | + | + | |
| + | + | | | + | | | | + | | + |
| + | + | | | + | | | | | + | + |
| + | + | | | | + | + | + | | | |
| + | + | | | | + | + | | + | | |
| + | + | | | | + | + | | | + | |
| + | + | | | | + | + | | | | + |
| + | + | | | | + | | + | + | | |
| + | + | | | | + | | + | | + | |
| + | + | | | | + | | + | | | + |
| + | + | | | | + | | | + | + | |
| + | + | | | | + | | | + | | + |
| + | + | | | | + | | | | + | + |
| + | + | | | | | + | + | + | | |
| + | + | | | | | + | + | | + | |
| + | + | | | | | + | + | | | + |
| + | + | | | | | + | | + | + | |
| + | + | | | | | + | | + | | + |
| + | + | | | | | + | | | + | + |
| + | + | | | | | | + | + | + | |
| + | + | | | | | | + | + | | + |
| + | + | | | | | | | + | + | + |
| + | | + | + | + | + | | | | | |
| + | | + | + | + | | + | | | | |
| + | | + | + | + | | | + | | | |
| + | | + | + | + | | | | + | | |
| + | | + | + | + | | | | | + | |
| + | | + | + | + | | | | | | + |
| + | | + | + | | + | + | | | | |
| + | | + | + | | + | | + | | | |
| + | | + | + | | + | | | + | | |
| + | | + | + | | + | | | | + | |
| + | | + | + | | + | | | | | + |
| + | | + | + | | + | | | | | |
| + | | + | + | | | + | + | | | |
| + | | + | + | | | + | | + | | |
| + | | + | + | | | + | | | + | |
| + | | + | + | | | + | | | | + |
| + | | + | + | | | | + | + | | |
| + | | + | + | | | | + | | + | |
| + | | + | + | | | | + | | | + |
| + | | + | + | | | | | + | + | |
| + | | + | + | | | | | + | | + |
| + | | + | + | | | | | | + | + |
| + | | + | | + | + | + | | | | |
| + | | + | | + | + | | + | | | |
| + | | + | | + | + | | | + | | |
| + | | + | | + | + | | | | + | |
| + | | + | | + | + | | | | | + |
| + | | + | | + | | + | + | | | |
| + | | + | | + | | + | | + | | |
| + | | + | | + | | + | | | + | |
| + | | + | | + | | + | | | | + |
| + | | + | | + | | | + | + | | |
| + | | + | | + | | | + | | + | |
| + | | + | | + | | | + | | | + |
| + | | + | | + | | | | + | + | |
| + | | + | | + | | | | + | | + |
| + | | + | | + | | | | | + | + |
| + | | + | | | + | + | + | | | |
| + | | + | | | + | + | | + | | |
| + | | + | | | + | + | | | + | |
| + | | + | | | + | + | | | | + |
| + | | + | | | + | | + | + | | |
| + | | + | | | + | | + | | + | |
| + | | + | | | + | | + | | | + |
| + | | + | | | + | | | + | + | |
| + | | + | | | + | | | + | | + |
| + | | + | | | + | | | | + | + |
| + | | + | | | | + | + | + | | |
| + | | + | | | | + | + | | + | |
| + | | + | | | | + | + | | | + |
| + | | + | | | | + | | + | + | |
| + | | + | | | | + | | + | | + |
| + | | + | | | | + | | | + | + |
| + | | + | | | | | + | + | + | |
| + | | + | | | | | + | + | | + |
| + | | + | | | | | + | | + | + |
| + | | + | | | | | | + | + | + |
| + | | | + | + | + | + | | | | |
| + | | | + | + | + | | + | | | |
| + | | | + | + | + | | | + | | |
| + | | | + | + | + | | | | + | |
| + | | | + | + | + | | | | | + |
| + | | | + | + | | + | + | | | |
| + | | | + | + | | + | | + | | |
| + | | | + | + | | + | | | + | |
| + | | | + | + | | + | | | | + |
| + | | | + | + | | | + | + | | |
| + | | | + | + | | | + | | + | |
| + | | | + | + | | | + | | | + |
| + | | | + | + | | | + | + | + | |
| + | | | + | + | | | + | + | + | + |
| + | | | + | + | | | + | + | | |
| + | | | + | + | | | + | | + | |
| + | | | + | + | | | + | | | + |
| + | | | + | + | | | | + | + | |
| + | | | + | + | | | | + | | + |
| + | | | + | + | | | | | + | + |
| + | | | + | | + | + | + | | | |
| + | | | + | | + | + | | + | | |
| + | | | + | | + | + | | | + | |
| + | | | + | | + | + | | | | + |
| + | | | + | | + | | + | + | | |
| + | | | + | | + | | + | | + | |
| + | | | + | | + | | + | | | + |
| + | | | + | | + | | | + | + | |
| + | | | + | | + | | | + | | + |
| + | | | + | | + | | | | + | + |
| + | | | + | | | + | + | + | | |
| + | | | + | | | + | + | | + | |
| + | | | + | | | + | + | | | + |
| + | | | + | | | + | | + | + | |
| + | | | + | | | + | | + | | + |
| + | | | + | | | + | | | + | + |
| + | | | + | | | | + | + | + | |
| + | | | + | | | | + | | | + |
| + | | | + | | | | | + | + | + |
| + | | | | + | + | + | + | | | |
| + | | | | + | + | + | | + | | |
| + | | | | + | + | + | | | + | |
| + | | | | + | + | + | | | | + |
| + | | | | + | + | | + | + | | |
| + | | | | + | + | | + | | + | |
| + | | | | + | + | | + | | | + |
| + | | | | + | + | | | + | + | |
| + | | | | + | + | | | + | | + |
| + | | | | + | + | | | | + | + |
| + | | | | + | | + | + | + | | |
| + | | | | + | | + | + | | + | |
| + | | | | + | | + | + | | | + |
| + | | | | + | | + | | + | + | |
| + | | | | + | | + | | + | | + |
| + | | | | + | | + | | | + | + |
| + | | | | + | | | + | + | + | |
| + | | | | + | | | + | + | | + |
| + | | | | + | | | + | | + | + |
| + | | | | + | | | | + | + | + |
| + | | | | | + | + | + | + | | |
| + | | | | | + | + | + | | + | |
| + | | | | | + | + | + | | | + |
| + | | | | | + | + | | + | + | |
| + | | | | | + | + | | + | | + |
| + | | | | | + | + | | | + | + |
| + | | | | | + | | + | + | + | |
| + | | | | | + | | + | + | | + |
| + | | | | | + | | + | | + | + |
| + | | | | | + | | | + | + | + |
| + | | | | | | + | + | + | + | |
| + | | | | | | + | + | + | | + |
| + | | | | | | + | + | | + | + |
| + | | | | | | | + | + | + | + |
| | + | + | + | + | + | | | | | |
| | + | + | + | + | | + | | | | |
| | + | + | + | + | | | + | | | |
| | + | + | + | + | | | | + | | |
| | + | + | + | + | | | | | + | |
| | + | + | + | + | | | | | | + |
| | + | + | + | | + | + | | | | |
| | + | + | + | | + | | + | | | |
| | + | + | + | | + | | | + | | |
| | + | + | + | | + | | | | + | |
| | + | + | + | | + | | | | | + |
| | + | + | + | | | + | + | | | |
| | + | + | + | | | + | | + | | |
| | + | + | + | | | + | | | + | |
| | + | + | + | | | + | | | | + |
| | + | + | + | | | | + | + | | |
| | + | + | + | | | | + | | + | |
| | + | + | + | | | | + | | | + |
| | + | + | + | | | | | + | + | |
| | + | + | + | | | | | + | | + |
| | + | + | + | | | | | | + | + |
| | + | + | | + | + | + | | | | |
| | + | + | | + | + | | + | | | |
| | + | + | | + | + | | | + | | |
| | + | + | | + | + | | | | + | |
| | + | + | | + | + | | | | | + |
| | + | + | | + | | + | + | | | |
| | + | + | | + | | + | | + | | |
| | + | + | | + | | + | | | + | |
| | + | + | | + | | + | | | | + |
| | + | + | | + | | | + | + | | |
| | + | + | | + | | | + | | + | |
| | + | + | | + | | | + | | | + |
| | + | + | | + | | | | + | + | |
| | + | + | | + | | | | + | | + |
| | + | + | | + | | | | | + | + |
| | + | + | | | + | + | + | | | |
| | + | + | | | + | + | | + | | |
| | + | + | | | + | + | | | + | |
| | + | + | | | + | + | | | | + |
| | + | + | | | + | | + | + | | |
| | + | + | | | + | | + | | + | |
| | + | + | | | + | | + | | | + |
| | + | + | | | + | | + | | | |
| | + | + | | | + | | | + | + | |
| | + | + | | | + | | | + | | + |
| | + | + | | | + | | | + | | |
| | + | + | | | + | | | | + | + |
| | + | + | | | | + | + | + | | |
| | + | + | | | | + | + | | + | |
| | + | + | | | | + | + | | | + |
| | + | + | | | | + | | + | + | |
| | + | + | | | | + | | + | | + |
| | + | + | | | | + | | | + | + |
| | + | + | | | | | + | + | + | |
| | + | + | | | | | + | + | | + |
| | + | + | | | | | + | | + | + |
| | + | + | | | | | | + | + | + |
| | + | | + | + | + | + | | | | |
| | + | | + | + | + | | + | | | |
| | + | | + | + | + | | | + | | |
| | + | | + | + | + | | | | + | |
| | + | | + | + | + | | | | | + |
| | + | | + | + | | + | + | | | |
| | + | | + | + | | + | | + | | |
| | + | | + | + | | + | | | + | |
| | + | | + | + | | + | | | | + |
| | + | | + | + | | | + | + | | |
| | + | | + | + | | | + | | + | |
| | + | | + | + | | | + | | | + |
| | + | | + | + | | | | + | + | |
| | + | | + | + | | | | + | | + |
| | + | | + | + | | | | | + | + |
| | + | | + | | + | + | + | | | |
| | + | | + | | + | + | | + | | |
| | + | | + | | + | + | | | + | |
| | + | | + | | + | + | | | | + |
| | + | | + | | + | | + | + | | |
| | + | | + | | + | | + | | + | |
| | + | | + | | + | | + | | | + |
| | + | | + | | + | | | + | + | |
| | + | | + | | + | | | + | | + |
| | + | | + | | + | | | | + | + |
| | + | | + | | | + | + | + | | |
| | + | | + | | | + | + | | + | |
| | + | | + | | | + | + | | | + |
| | + | | + | | | + | | + | + | |
| | + | | + | | | + | | + | | + |
| | + | | + | | | + | | | + | + |
| | + | | + | | | | + | + | + | |
| | + | | + | | | | + | + | | + |
| | + | | + | | | | + | | + | + |
| | + | | + | | | | | + | + | + |
| | + | | | + | + | + | + | | | |
| | + | | | + | + | + | | + | | |
| | + | | | + | + | + | | | + | |
| | + | | | + | + | + | | | | + |
| | + | | | + | + | | + | + | | |
| | + | | | + | + | | + | | + | |
| | + | | | + | + | | + | | | + |
| | + | | | + | + | | | + | + | |
| | + | | | + | + | | | + | | + |
| | + | | | + | + | | | | + | + |
| | + | | | + | | + | + | + | | |
| | + | | | + | | + | + | | + | |
| | + | | | + | | + | + | | | + |
| | + | | | + | | + | | + | + | |
| | + | | | + | | + | | + | | + |
| | + | | | + | | + | | | + | + |
| | + | | | + | | | + | + | + | |
| | + | | | + | | | + | + | | + |
| | + | | | + | | | + | | + | + |
| | + | | | + | | | | + | + | + |
| | + | | | | + | + | + | + | | |
| | + | | | | + | + | + | | + | |
| | + | | | | + | + | + | | | + |
| | + | | | | + | + | | + | + | |
| | + | | | | + | + | | + | | + |
| | + | | | | + | + | | | + | + |
| | + | | | | + | | + | + | + | |
| | + | | | | + | | + | + | | + |
| | + | | | | + | | + | | + | + |
| | + | | | | + | | | + | + | + |
| | + | | | | | + | + | + | + | |
| | + | | | | | + | + | + | | + |
| | + | | | | | + | + | | + | + |
| | + | | | | | + | | + | + | + |
| | + | | | | | | + | + | + | + |
| | | + | + | + | + | + | | | | |
| | | + | + | + | + | | + | | | |
| | | + | + | + | + | | | + | | |
| | | + | + | + | + | | | | + | |
| | | + | + | + | + | | | | | + |
| | | + | + | + | | + | + | | | |
| | | + | + | + | | + | | + | | |
| | | + | + | + | | + | | | + | |
| | | + | + | + | | + | | | | + |
| | | + | + | + | | | + | + | | |
| | | + | + | + | | | + | | + | |
| | | + | + | + | | | + | | | + |
| | | + | + | | + | + | + | | | |
| | | + | + | | + | + | | + | | |
| | | + | + | | + | + | | | + | |
| | | + | + | | + | + | | | | + |
| | | + | + | | + | | + | + | | |
| | | + | + | | + | | + | | + | |
| | | + | + | | + | | + | | | + |
| | | + | + | | + | | | + | + | |
| | | + | + | | + | | | + | | + |
| | | + | + | | + | | | | + | + |
| | | + | + | | | + | + | + | | |
| | | + | + | | | + | + | | + | |
| | | + | + | | | + | + | | | + |
| | | + | + | | | + | | + | + | |
| | | + | + | | | + | | + | | + |
| | | + | + | | | + | | | + | + |
| | | + | + | | | | + | + | + | |
| | | + | + | | | | + | + | | + |
| | | + | + | | | | + | | + | + |
| | | + | + | | | | | + | + | + |
| | | + | | + | + | + | + | | | |
| | | + | | + | + | + | | + | | |
| | | + | | + | + | + | | | + | |
| | | + | | + | + | + | | | | + |
| | | + | | + | + | | + | + | | |
| | | + | | + | + | | + | | + | |
| | | + | | + | + | | + | | | + |
| | | + | | + | + | | | + | + | |
| | | + | | + | + | | | + | | + |
| | | + | | + | + | | | | + | + |
| | | + | | + | | + | + | + | | |
| | | + | | + | | + | + | | + | |
| | | + | | + | | + | + | | | + |
| | | + | | + | | + | | + | + | |
| | | + | | + | | + | | + | | + |
| | | + | | + | | + | | | + | + |
| | | + | | + | | | + | + | + | |
| | | + | | + | | | + | + | | + |
| | | + | | + | | | + | | + | + |
| | | + | | + | | | | + | + | + |
| | | + | | | + | + | + | + | | |
| | | + | | | + | + | + | | + | |
| | | + | | | + | + | + | | | + |
| | | + | | | + | + | | + | + | |
| | | + | | | + | + | | + | | + |
| | | + | | | + | + | | | + | + |
| | | + | | | + | | + | + | + | |
| | | + | | | + | | + | + | | + |
| | | + | | | + | | + | | + | + |
| | | + | | | + | | | + | + | + |
| | | + | | | | + | + | + | + | |
| | | + | | | | + | + | + | | + |
| | | + | | | | + | + | | + | + |
| | | + | | | | + | | + | + | + |
| | | + | | | | | + | + | + | + |
| | | | + | + | + | + | + | | | |
| | | | + | + | + | + | | + | | |
| | | | + | + | + | + | | | + | |
| | | | + | + | + | + | | | | + |
| | | | + | + | + | | + | + | | |
| | | | + | + | + | | + | | + | |
| | | | + | + | + | | + | | | + |
| | | | + | + | + | | | + | + | |
| | | | + | + | + | | | + | | + |
| | | | + | + | + | | | | + | + |
| | | | + | + | | + | + | + | | |
| | | | + | + | | + | + | | + | |
| | | | + | + | | + | + | | | + |
| | | | + | + | | + | | + | + | |
| | | | + | + | | + | | + | | + |
| | | | + | + | | + | | | + | + |
| | | | + | + | | | + | + | + | |
| | | | + | + | | | + | + | | + |
| | | | + | + | | | + | | + | + |
| | | | + | + | | | | + | + | + |
| | | | + | | + | + | + | + | | |
| | | | + | | + | + | + | | + | |
| | | | + | | + | + | + | | | + |
| | | | + | | + | + | | + | + | |
| | | | + | | + | + | | + | | + |
| | | | + | | + | + | | | + | + |
| | | | + | | | + | + | + | + | |
| | | | + | | | + | + | + | | + |
| | | | + | | | + | + | | + | + |
| | | | + | | | + | | + | + | + |
| | | | + | | | | + | + | + | + |
| | | | + | | | | + | + | + | + |
| | | | | + | + | + | + | + | | |
| | | | | + | + | + | + | | + | |
| | | | | + | + | + | + | | | + |
| | | | | + | + | + | | + | + | |
| | | | | + | + | + | | + | | + |
| | | | | + | + | + | | | + | + |
| | | | | + | + | | + | + | + | |
| | | | | + | + | | + | + | | + |
| | | | | + | + | | + | | + | + |
| | | | | + | + | | | + | + | + |
| | | | | + | | + | + | + | + | |
| | | | | + | | + | + | + | | + |
| | | | | + | | + | + | | + | + |
| | | | | + | | + | | + | + | + |
| | | | | + | | | + | + | + | + |
| | | | | | + | + | + | + | + | |
| | | | | | + | + | + | + | | + |
| | | | | | + | + | + | | + | + |
| | | | | | + | + | | + | + | + |
| | | | | | + | | + | + | + | + |
| | | | | | | + | + | + | + | + |
| + | + | + | + | + | + | | | | | |
| + | + | + | + | + | | + | | | | |
| + | + | + | + | + | | | + | | | |
| + | + | + | + | + | | | | + | | |
| + | + | + | + | + | | | | | + | |
| + | + | + | + | + | | | | | | + |
| + | + | + | + | | + | + | | | | |
| + | + | + | + | | + | | + | | | |
| + | + | + | + | | + | | | + | | |
| + | + | + | + | | + | | | | + | |
| + | + | + | + | | + | | | | | + |
| + | + | + | + | | | + | + | | | |
| + | + | + | + | | | + | | + | | |
| + | + | + | + | | | + | | | + | |
| + | + | + | + | | | + | | | | + |
| + | + | + | + | | | | + | + | | |
| + | + | + | + | | | | + | | + | |
| + | + | + | + | | | | + | | | + |
| + | + | + | + | | | | | + | + | + |
| + | + | + | | + | + | + | | | | |
| + | + | + | | + | + | | + | | | |
| + | + | + | | + | + | | | + | | |
| + | + | + | | + | + | | | | + | |
| + | + | + | | + | + | | | | | + |
| + | + | + | | + | | + | + | | | |
| + | + | + | | + | | + | | + | | |
| + | + | + | | + | | + | | | + | |
| + | + | + | | + | | + | | | | + |
| + | + | + | | + | | | + | + | | |
| + | + | + | | + | | | + | | + | |
| + | + | + | | + | | | + | | | + |
| + | + | + | | + | | | | + | + | |
| + | + | + | | + | | | | + | | + |
| + | + | + | | + | | | | | + | + |
| + | + | + | | | + | + | + | | | |
| + | + | + | | | + | + | | + | | |
| + | + | + | | | + | + | | | + | |
| + | + | + | | | + | + | | | | + |
| + | + | + | | | + | + | | | | |
| + | + | + | | | + | | + | + | | |
| + | + | + | | | + | | + | | + | |
| + | + | + | | | + | | + | | | + |
| + | + | + | | | + | | | + | + | |
| + | + | + | | | + | | | + | | + |
| + | + | + | | | + | | | | + | + |
| + | + | + | | | | + | + | + | | |
| + | + | + | | | | + | + | | + | |
| + | + | + | | | | + | + | | | + |
| + | + | + | | | | + | | + | + | |
| + | + | + | | | | + | | + | | + |
| + | + | + | | | | + | | | + | + |
| + | + | + | | | | | + | + | + | |
| + | + | + | | | | | + | + | | + |
| + | + | + | | | | | + | | + | + |
| + | + | + | | | | | | + | + | + |
| + | + | | + | + | + | + | | | | |
| + | + | | + | + | + | | + | | | |
| + | + | | + | + | + | | | + | | |
| + | + | | + | + | + | | | | + | |
| + | + | | + | + | + | | | | | + |
| + | + | | + | + | | + | + | | | |
| + | + | | + | + | | + | | + | | |
| + | + | | + | + | | + | | | + | |
| + | + | | + | + | | + | | | | + |
| + | + | | + | + | | | + | + | | |
| + | + | | + | + | | | + | | + | |
| + | + | | + | + | | | + | | | + |
| + | + | | + | + | | | | + | + | |
| + | + | | + | + | | | | + | | + |
| + | + | | + | + | | | | | + | + |
| + | + | | + | | + | + | + | | | |
| + | + | | + | | + | + | | + | | |
| + | + | | + | | + | + | | | + | |
| + | + | | + | | + | + | | | | + |
| + | + | | + | | + | | + | + | | |
| + | + | | + | | + | | + | | + | |
| + | + | | + | | + | | + | | | + |
| + | + | | + | | + | | | + | + | |
| + | + | | + | | + | | | | + | + |
| + | + | | + | | | + | + | + | | |
| + | + | | + | | | + | + | | + | |
| + | + | | + | | | + | + | | | + |
| + | + | | + | | | + | | + | + | |
| + | + | | + | | | + | | + | | + |
| + | + | | + | | | + | | | + | + |
| + | + | | | + | + | + | + | | | |
| + | + | | | + | + | + | | + | | |
| + | + | | | + | + | + | | | + | |
| + | + | | | + | + | + | | | | + |
| + | + | | | + | + | | + | + | | |
| + | + | | | + | + | | + | | + | |
| + | + | | | + | + | | + | | | + |
| + | + | | | + | + | | | + | + | |
| + | + | | | + | + | | | + | | + |
| + | + | | | + | + | | | + | | |
| + | + | | | + | + | | | | + | + |
| + | + | | | + | | + | + | + | | |
| + | + | | | + | | + | + | | + | |
| + | + | | | + | | + | + | | | + |
| + | + | | | + | | + | | + | + | |
| + | + | | | + | | + | | + | | + |
| + | + | | | + | | + | | | + | + |
| + | + | | | + | | | + | + | + | |
| + | + | | | + | | | + | + | | + |
| + | + | | | + | | | + | | + | + |
| + | + | | | + | | | | + | + | + |
| + | + | | | | + | + | + | + | | |
| + | + | | | | + | + | + | | + | |
| + | + | | | | + | + | + | | | + |
| + | + | | | | + | + | | + | + | |
| + | + | | | | + | + | | + | | + |
| + | + | | | | + | + | | | + | + |
| + | + | | | | | + | + | + | + | |
| + | + | | | | | + | + | + | | + |
| + | + | | | | | + | + | | + | + |
| + | + | | | | | + | | + | + | + |
| + | + | | | | | | + | + | + | + |
| + | | + | + | + | + | + | | | | |
| + | | + | + | + | + | | + | | | |
| + | | + | + | + | + | | | + | | |
| + | | + | + | + | + | | | | + | |
| + | | + | + | + | + | | | | | + |
| + | | + | + | + | | + | + | | | |
| + | | + | + | + | | + | | + | | |
| + | | + | + | + | | + | | | + | |
| + | | + | + | + | | + | | | | + |
| + | | + | + | + | | | + | + | | |
| + | | + | + | + | | | + | | + | |
| + | | + | + | + | | | + | | | + |
| + | | + | + | + | | | | + | + | |
| + | | + | + | + | | | | + | | + |
| + | | + | + | + | | | | | + | + |
| + | | + | + | | + | + | + | | | |
| + | | + | + | | + | + | | + | | |
| + | | + | + | | + | + | | | + | |
| + | | + | + | | + | + | | | | + |
| + | | + | + | | + | | + | + | | |
| + | | + | + | | + | | + | | + | |
| + | | + | + | | + | | + | | | + |
| + | | + | + | | + | | | + | + | |
| + | | + | + | | + | | | + | | + |
| + | | + | + | | + | | | | + | + |
| + | | + | + | | | + | + | + | | |
| + | | + | + | | | + | + | | + | |
| + | | + | + | | | + | + | | | + |
| + | | + | + | | | + | | + | + | |
| + | | + | + | | | + | | + | | + |
| + | | + | + | | | + | | | + | + |
| + | | + | + | | | | + | + | + | |
| + | | + | + | | | | + | + | | + |
| + | | + | + | | | | | + | + | + |
| + | | + | | + | + | + | + | | | |
| + | | + | | + | + | + | | + | | |
| + | | + | | + | + | + | | | + | |
| + | | + | | + | + | + | | | | + |
| + | | + | | + | + | | + | + | | |
| + | | + | | + | + | | + | | + | |
| + | | + | | + | + | | + | | | + |
| + | | + | | + | + | | | + | + | |
| + | | + | | + | + | | | + | | + |
| + | | + | | + | + | | | | + | + |
| + | | + | | + | | + | + | + | | |
| + | | + | | + | | + | + | | + | |
| + | | + | | + | | + | + | | | + |
| + | | + | | + | | | | + | + | |
| + | | + | | + | | | | + | | + |
| + | | + | | + | | | | | + | + |
| + | | + | | | + | + | + | + | | |
| + | | + | | | + | + | + | | + | |
| + | | + | | | + | + | + | | | + |
| + | | + | | | + | + | | + | + | |
| + | | + | | | + | + | | + | | + |
| + | | + | | | + | + | | | + | + |
| + | | + | | | + | | + | + | + | |
| + | | + | | | + | | + | + | | + |
| + | | + | | | + | | + | + | | |
| + | | + | | | + | | + | | + | + |
| + | | + | | | + | | | + | + | + |
| + | | + | | | | + | + | + | + | |
| + | | + | | | | + | + | + | | + |
| + | | + | | | | + | + | | + | + |
| + | | + | | | | + | | + | + | + |
| + | | + | | | | | + | + | + | + |
| + | | | + | + | + | + | + | | | |
| + | | | + | + | + | + | | + | | |
| + | | | + | + | + | + | | | + | |
| + | | | + | + | + | + | | | | + |
| + | | | + | + | + | | + | + | | |
| + | | | + | + | + | | + | | + | |
| + | | | + | + | + | | + | | | + |
| + | | | + | + | + | | | + | + | |
| + | | | + | + | + | | | + | | + |
| + | | | + | + | + | | | | + | + |
| + | | | + | + | | + | + | + | | |
| + | | | + | + | | + | + | | + | |
| + | | | + | + | | + | + | | | + |
| + | | | + | + | | + | | + | + | |
| + | | | + | + | | + | | + | | + |
| + | | | + | + | | + | | | + | + |
| + | | | + | + | | | + | + | + | |
| + | | | + | + | | | + | + | | + |
| + | | | + | + | | | + | | + | + |
| + | | | + | + | | | | + | + | + |
| + | | | + | | + | + | + | + | | |
| + | | | + | | + | + | + | | + | |
| + | | | + | | + | + | + | | | + |
| + | | | + | | + | + | | + | + | |
| + | | | + | | + | + | | + | | + |
| + | | | + | | + | + | | | + | + |
| + | | | + | | + | | + | + | + | |
| + | | | + | | + | | + | + | | + |
| + | | | + | | + | | + | | + | + |
| + | | | + | | + | | | + | + | + |
| + | | | + | | | + | + | + | + | |
| + | | | + | | | + | + | + | | + |
| + | | | + | | | + | + | | + | + |
| + | | | + | | | + | | + | + | + |
| + | | | + | | | | + | + | + | + |
| + | | | | + | + | + | + | + | | |
| + | | | | + | + | + | + | | + | |
| + | | | | + | + | + | + | | | + |
| + | | | | + | + | + | | + | + | |
| + | | | | + | + | + | | + | | + |
| + | | | | + | + | + | | | + | + |
| + | | | | + | + | | + | + | + | |
| + | | | | + | + | | + | + | | + |
| + | | | | + | + | | + | | + | + |
| + | | | | + | + | | | + | + | + |
| + | | | | + | | | + | + | + | + |
| + | | | | | + | + | + | + | + | |
| + | | | | | + | + | + | + | | + |
| + | | | | | + | + | + | | + | + |
| + | | | | | + | + | | + | + | + |
| + | | | | | + | | + | + | + | + |
| + | | | | | | + | + | + | + | + |
| | + | + | + | + | + | + | | | | |
| | + | + | + | + | + | | + | | | |
| | + | + | + | + | + | | | + | | |
| | + | + | + | + | + | | | | + | |
| | + | + | + | + | + | | | | | + |
| | + | + | + | + | | + | + | | | |
| | + | + | + | + | | + | | + | | |
| | + | + | + | + | | + | | | + | |
| | + | + | + | + | | + | | | | + |
| | + | + | + | + | | | + | + | | |
| | + | + | + | + | | | + | | + | |
| | + | + | + | + | | | + | | | + |
| | + | + | + | + | | | | + | + | |
| | + | + | + | + | | | | + | | + |
| | + | + | + | + | | | | | + | + |
| | + | + | + | | + | + | + | | | |
| | + | + | + | | + | + | | + | | |
| | + | + | + | | + | + | | | + | |
| | + | + | + | | + | + | | | | + |
| | + | + | + | | + | | + | + | | |
| | + | + | + | | + | | + | | + | |
| | + | + | + | | + | | + | | | + |
| | + | + | + | | + | | | + | + | |
| | + | + | + | | + | | | + | | + |
| | + | + | + | | + | | | | + | + |
| | + | + | + | | | + | + | + | | |
| | + | + | + | | | + | + | | + | |
| | + | + | + | | | + | + | | | + |
| | + | + | + | | | + | + | | | |
| | + | + | + | | | + | | + | + | |
| | + | + | + | | | + | | + | | + |
| | + | + | + | | | + | | + | | |
| | + | + | + | | | + | | | + | + |
| | + | + | + | | | | + | + | + | |
| | + | + | + | | | | + | + | | + |
| | + | + | + | | | | + | + | | |
| | + | + | + | | | | + | + | | |
| | + | + | + | | | | + | | + | + |
| | + | + | + | | | | | + | + | + |
| | + | + | | + | + | + | + | | | |
| | + | + | | + | + | + | | + | | |
| | + | + | | + | + | + | | | + | |
| | + | + | | + | + | + | | | | + |
| | + | + | | + | + | | + | + | | |
| | + | + | | + | + | | + | | + | |
| | + | + | | + | + | | + | | | + |
| | + | + | | + | + | | | + | + | |
| | + | + | | + | + | | | + | | + |
| | + | + | | + | + | | | | + | + |
| | + | + | | + | | + | + | + | | |
| | + | + | | + | | + | + | | + | |
| | + | + | | + | | + | + | | | + |
| | + | + | | + | | + | | + | + | |
| | + | + | | + | | + | | + | | + |
| | + | + | | + | | + | | | + | + |
| | + | + | | + | | | + | + | + | |
| | + | + | | + | | | + | + | | + |
| | + | + | | + | | | + | | + | + |
| | + | + | | + | | | | + | + | + |
| | + | + | | | + | + | + | + | | |
| | + | + | | | + | + | + | | + | |
| | + | + | | | + | + | + | | | + |
| | + | + | | | + | + | | + | + | |
| | + | + | | | + | + | | + | | + |
| | + | + | | | + | + | | | + | + |
| | + | + | | | + | | + | + | + | |
| | + | + | | | + | | + | + | | + |
| | + | + | | | + | | + | | + | + |
| | + | + | | | + | | | + | + | + |
| | + | + | | | | + | + | + | + | |
| | + | + | | | | + | + | + | | + |
| | + | + | | | | + | + | | + | + |
| | + | + | | | | | + | + | + | + |
| | + | | + | + | + | + | + | | | |
| | + | | + | + | + | + | | + | | |
| | + | | + | + | + | + | | | + | |
| | + | | + | + | + | + | | | | + |
| | + | | + | + | + | | + | + | | |
| | + | | + | + | + | | + | | + | |
| | + | | + | + | + | | + | | | + |
| | + | | + | + | + | | | + | + | |
| | + | | + | + | + | | | + | | + |
| | + | | + | + | + | | | | + | + |
| | + | | + | + | | + | + | + | | |
| | + | | + | + | | + | + | | + | |
| | + | | + | + | | + | + | | | + |
| | + | | + | + | | + | | + | + | |
| | + | | + | + | | + | | + | | + |
| | + | | + | + | | + | | | + | + |
| | + | | + | + | | | + | + | + | |
| | + | | + | + | | | + | + | | + |
| | + | | + | + | | | + | | + | + |
| | + | | + | + | | | | + | + | + |
| | + | | + | | + | + | + | + | | |
| | + | | + | | + | + | + | | + | |
| | + | | + | | + | + | + | | | + |
| | + | | + | | + | + | | + | + | |
| | + | | + | | + | + | | + | | + |
| | + | | + | | + | + | | | + | + |
| | + | | + | | + | | + | + | + | |
| | + | | + | | + | | + | + | | + |
| | + | | + | | + | | + | | + | + |
| | + | | + | | + | | | + | + | + |
| | + | | + | | | + | + | + | + | |
| | + | | + | | | + | + | + | | + |
| | + | | + | | | + | + | | + | + |
| | + | | + | | | + | | + | + | + |
| | + | | + | | | | + | + | + | + |
| | + | | | + | + | + | + | + | | |
| | + | | | + | + | + | + | | + | |
| | + | | | + | + | + | + | | | + |
| | + | | | + | + | + | | + | + | |
| | + | | | + | + | + | | + | | + |
| | + | | | + | + | + | | | + | + |
| | + | | | + | + | | + | + | + | |
| | + | | | + | + | | + | + | | + |
| | + | | | + | + | | + | | + | + |
| | + | | | + | + | | | + | + | + |
| | + | | | + | | + | + | + | + | |
| | + | | | + | | + | + | + | | + |
| | + | | | + | | + | + | | + | + |
| | + | | | + | | + | | + | + | + |
| | + | | | + | | | + | + | + | + |
| | + | | | | + | + | + | + | + | |
| | + | | | | + | + | + | + | | + |
| | + | | | | + | + | + | | + | + |
| | + | | | | + | + | | + | + | + |
| | + | | | | + | | + | + | + | + |
| | + | | | | | + | + | + | + | + |
| | | + | + | + | + | + | + | | | |
| | | + | + | + | + | + | | + | | |
| | | + | + | + | + | + | | | + | |
| | | + | + | + | + | + | | | | + |
| | | + | + | + | + | | + | + | | |
| | | + | + | + | + | | + | | + | |
| | | + | + | + | + | | + | | | + |
| | | + | + | + | + | | | + | + | |
| | | + | + | + | + | | | + | | + |
| | | + | + | + | + | | | | + | + |
| | | + | + | + | | + | + | + | | |
| | | + | + | + | | + | + | | + | |
| | | + | + | + | | + | + | | | + |
| | | + | + | + | | + | | + | + | |
| | | + | + | + | | + | | + | | + |
| | | + | + | + | | + | | | + | + |
| | | + | + | + | | | + | + | + | |
| | | + | + | + | | | + | + | | + |
| | | + | + | + | | | + | | + | + |
| | | + | + | + | | | | + | + | + |
| | | + | + | | + | + | + | + | | |
| | | + | + | | + | + | + | | + | |
| | | + | + | | + | + | + | | | + |
| | | + | + | | + | + | | + | + | |
| | | + | + | | + | + | | + | | + |
| | | + | + | | + | + | | | + | + |
| | | + | + | | + | | + | + | + | |
| | | + | + | | + | | + | + | | + |
| | | + | + | | + | | + | | + | + |
| | | + | + | | + | | | + | + | + |
| | | + | + | | | + | + | + | + | |
| | | + | + | | | + | + | + | | + |
| | | + | + | | | + | + | | + | + |
| | | + | + | | | + | | + | + | + |
| | | + | + | | | | + | + | + | + |
| | | + | | + | + | + | + | + | | |
| | | + | | + | + | + | + | | + | |
| | | + | | + | + | + | + | | | + |
| | | + | | + | + | + | | + | + | |
| | | + | | + | + | + | | + | | + |
| | | + | | + | + | + | | | + | + |
| | | + | | + | + | | + | + | + | |
| | | + | | + | + | | + | + | | + |
| | | + | | + | + | | + | | + | + |
| | | + | | + | + | | | + | + | + |
| | | + | | + | | + | + | + | + | |
| | | + | | + | | + | + | + | | + |
| | | + | | + | | + | + | | + | + |
| | | + | | + | | + | | + | + | + |
| | | + | | + | | | + | + | + | + |
| | | + | | | + | + | + | + | + | |
| | | + | | | + | + | + | + | | + |
| | | + | | | + | + | + | | + | + |
| | | + | | | + | + | | + | + | + |
| | | + | | | + | | + | + | + | + |
| | | + | | | | + | + | + | + | + |
| | | | + | + | + | + | + | + | | |
| | | | + | + | + | + | + | | + | |
| | | | + | + | + | + | + | | | + |
| | | | + | + | + | + | | + | + | |
| | | | + | + | + | + | | + | | + |
| | | | + | + | + | + | | | + | + |
| | | | + | + | + | | + | + | + | |
| | | | + | + | + | | + | + | | + |
| | | | + | + | + | | + | | + | + |
| | | | + | + | + | | | + | + | + |
| | | | + | + | | + | + | + | + | |
| | | | + | + | | + | + | + | | + |
| | | | + | + | | + | + | | + | + |
| | | | + | + | | + | | + | + | + |
| | | | + | + | | | + | + | + | + |
| | | | + | | + | + | + | + | + | |
| | | | + | | + | + | + | + | | + |
| | | | + | | + | + | + | | + | + |
| | | | + | | + | + | | + | + | + |
| | | | + | | + | | + | + | + | + |
| | | | + | | | + | + | + | + | + |
| | | | | + | + | + | + | + | + | |
| | | | | + | + | + | + | + | | + |
| | | | | + | + | + | + | | + | + |
| | | | | + | + | + | | + | + | + |
| | | | | + | + | | + | + | + | + |
| | | | | + | | + | + | + | + | + |
| | | | | | + | + | + | + | + | + |
| + | + | + | + | + | + | + | | | | |
| + | + | + | + | + | + | | + | | | |
| + | + | + | + | + | + | | | + | | |
| + | + | + | + | + | + | | | | + | |
| + | + | + | + | + | + | | | | | + |
| + | + | + | + | + | | + | + | | | |
| + | + | + | + | + | | + | | + | | |
| + | + | + | + | + | | + | | | + | |
| + | + | + | + | + | | + | | | | + |
| + | + | + | + | + | | | + | + | | |
| + | + | + | + | + | | | + | | + | |
| + | + | + | + | + | | | + | | | + |
| + | + | + | + | + | | | | + | + | |
| + | + | + | + | + | | | | + | | + |
| + | + | + | + | + | | | | | + | + |
| + | + | + | + | | + | + | + | | | |
| + | + | + | + | | + | + | | + | | |
| + | + | + | + | | + | + | | | + | |
| + | + | + | + | | + | + | | | | + |
| + | + | + | + | | + | | + | + | | |
| + | + | + | + | | + | | + | | + | |
| + | + | + | + | | + | | + | | | + |
| + | + | + | + | | + | | | + | + | |
| + | + | + | + | | + | | | + | | + |
| + | + | + | + | | + | | | | + | + |
| + | + | + | + | | | + | + | + | | |
| + | + | + | + | | | + | + | | + | |
| + | + | + | + | | | + | + | | | + |
| + | + | + | + | | | + | | + | + | |
| + | + | + | + | | | + | | + | | + |
| + | + | + | + | | | + | | | + | + |
| + | + | + | + | | | | + | + | + | |
| + | + | + | + | | | | + | + | | + |
| + | + | + | + | | | | | + | + | + |
| + | + | + | | + | + | + | + | | | |
| + | + | + | | + | + | + | | + | | |
| + | + | + | | + | + | + | | | + | |
| + | + | + | | + | + | + | | | | + |
| + | + | + | | + | + | | + | + | | |
| + | + | + | | + | + | | + | | + | |
| + | + | + | | + | + | | + | | | + |
| + | + | + | | + | + | | | + | + | |
| + | + | + | | + | + | | | + | | + |
| + | + | + | | + | + | | | | + | + |
| + | + | + | | + | | + | + | + | | |
| + | + | + | | + | | + | + | | + | |
| + | + | + | | + | | + | + | | | + |
| + | + | + | | + | | + | | + | + | |
| + | + | + | | + | | + | | + | | + |
| + | + | + | | + | | + | | | + | + |
| + | + | + | | + | | | + | + | + | |
| + | + | + | | + | | | + | + | | + |
| + | + | + | | + | | | + | | + | + |
| + | + | + | | + | | | | + | + | + |
| + | + | + | | | + | + | + | + | | |
| + | + | + | | | + | + | + | | + | |
| + | + | + | | | + | + | + | | | + |
| + | + | + | | | + | + | | + | + | |
| + | + | + | | | + | + | | + | | + |
| + | + | + | | | + | + | | | + | + |
| + | + | + | | | + | | | + | + | + |
| + | + | + | | | | + | + | + | + | |
| + | + | + | | | | + | + | + | | + |
| + | + | + | | | | + | + | | + | + |
| + | + | + | | | | + | | + | + | + |
| + | + | + | | | | | + | + | + | + |
| + | + | | + | + | + | + | + | | | |
| + | + | | + | + | + | + | | + | | |
| + | + | | + | + | + | + | | | + | |
| + | + | | + | + | + | + | | | | + |
| + | + | | + | + | + | | + | + | | |
| + | + | | + | + | + | | + | | + | |
| + | + | | + | + | + | | + | | | + |
| + | + | | + | + | + | | | + | + | |
| + | + | | + | + | + | | | + | | + |
| + | + | | + | + | + | | | | + | + |
| + | + | | + | + | | + | + | + | | |
| + | + | | + | + | | + | + | | + | |
| + | + | | + | + | | + | + | | | + |
| + | + | | + | + | | + | | + | + | |
| + | + | | + | + | | + | | + | | + |
| + | + | | + | + | | + | | | + | + |
| + | + | | + | + | | | + | + | + | |
| + | + | | + | + | | | + | + | | + |
| + | + | | + | + | | | + | | + | + |
| + | + | | + | + | | | | + | + | + |
| + | + | | + | | + | + | + | + | | |
| + | + | | + | | + | + | + | | + | |
| + | + | | + | | + | + | + | | | + |
| + | + | | + | | + | + | | + | + | |
| + | + | | + | | + | + | | + | | + |
| + | + | | + | | + | + | | | + | + |
| + | + | | + | | + | | + | + | + | |
| + | + | | + | | + | | + | + | | + |
| + | + | | + | | + | | + | | + | + |
| + | + | | + | | + | | | + | + | + |
| + | + | | + | | | + | + | + | + | |
| + | + | | + | | | + | + | + | | + |
| + | + | | + | | | + | + | | + | + |
| + | + | | + | | | + | | + | + | + |
| + | + | | + | | | | + | + | + | + |
| + | + | | | + | + | + | + | + | | |
| + | + | | | + | + | + | + | | + | |
| + | + | | | + | + | + | + | | | + |
| + | + | | | + | + | + | | + | + | |
| + | + | | | + | + | + | | + | | + |
| + | + | | | + | + | + | | | + | + |
| + | + | | | | + | + | + | + | + | |
| + | + | | | | + | + | + | + | | + |
| + | + | | | | + | + | | + | + | + |
| + | + | | | | + | | + | + | + | + |
| + | + | | | | | + | + | + | + | + |
| + | | + | + | + | + | + | + | | | |
| + | | + | + | + | + | + | | + | | |
| + | | + | + | + | + | + | | | + | |
| + | | + | + | + | + | + | | | | + |
| + | | + | + | + | + | | + | + | | |
| + | | + | + | + | + | | + | | + | |
| + | | + | + | + | + | | + | | | + |
| + | | + | + | + | + | | | + | + | |
| + | | + | + | + | + | | | + | | + |
| + | | + | + | + | + | | | | + | + |
| + | | + | + | + | | + | + | + | | |
| + | | + | + | + | | + | + | | + | |
| + | | + | + | + | | + | + | | | + |
| + | | + | + | + | | + | | + | + | |
| + | | + | + | + | | + | | + | | + |
| + | | + | + | + | | + | | | + | + |
| + | | + | + | + | | | + | + | + | |
| + | | + | + | + | | | + | + | | + |
| + | | + | + | + | | | + | | + | + |
| + | | + | + | + | | | | + | + | + |
| + | | + | + | | + | + | + | + | | |
| + | | + | + | | + | + | + | | + | |
| + | | + | + | | + | + | + | | | + |
| + | | + | + | | + | + | | + | + | |
| + | | + | + | | + | + | | + | | + |
| + | | + | + | | + | + | | | + | + |
| + | | + | + | | + | | + | + | + | |
| + | | + | + | | + | | + | + | | + |
| + | | + | + | | + | | + | | + | + |
| + | | + | + | | + | | | + | + | + |
| + | | + | + | | | + | + | + | + | |
| + | | + | + | | | + | + | + | | + |
| + | | + | + | | | + | + | | + | + |
| + | | + | + | | | + | | + | + | + |
| + | | + | + | | | | + | + | + | + |
| + | | + | | + | + | + | + | + | | |
| + | | + | | + | + | + | + | | + | |
| + | | + | | + | + | + | + | | | + |
| + | | + | | + | + | + | | + | + | |
| + | | + | | + | + | + | | + | | + |
| + | | + | | + | + | + | | | + | + |
| + | | + | | + | + | | + | + | + | |
| + | | + | | + | + | | + | + | | + |
| + | | + | | + | + | | + | | + | + |
| + | | + | | + | + | | | + | + | + |
| + | | + | | + | | + | + | + | + | |
| + | | + | | + | | + | + | + | | + |
| + | | + | | + | | + | + | | + | + |
| + | | + | | + | | + | | + | + | + |
| + | | + | | + | | | + | + | + | + |
| + | | + | | | + | + | + | + | + | |
| + | | + | | | + | + | + | + | | + |
| + | | + | | | + | + | + | | + | + |
| + | | + | | | + | + | | + | + | + |
| + | | + | | | + | | + | + | + | + |
| + | | + | | | | + | + | + | + | + |
| + | | | + | + | + | + | + | + | | |
| + | | | + | + | + | + | + | | + | |
| + | | | + | + | + | + | + | | | + |
| + | | | + | + | + | + | | + | + | |
| + | | | + | + | + | + | | + | | + |
| + | | | + | + | + | + | | | + | + |
| + | | | + | + | + | | + | + | + | |
| + | | | + | + | + | | + | + | | + |
| + | | | + | + | + | | + | | + | + |
| + | | | + | + | | + | + | + | + | |
| + | | | + | + | | + | + | + | | + |
| + | | | + | + | | + | + | | + | + |
| + | | | + | + | | + | | + | + | + |
| + | | | + | + | | | + | + | + | + |
| + | | | + | | + | + | + | + | + | |
| + | | | + | | + | + | + | + | | + |
| + | | | + | | + | + | + | | + | + |
| + | | | + | | + | + | | + | + | + |
| + | | | + | | + | | + | + | + | + |
| + | | | + | | | + | + | + | + | + |
| + | | | | + | + | + | + | + | + | |
| + | | | | + | + | + | + | + | | + |
| + | | | | + | + | + | + | | + | + |
| + | | | | + | + | + | | + | + | + |
| + | | | | + | + | | + | + | + | + |
| + | | | | + | | + | + | + | + | + |
| + | | | | | + | + | + | + | + | + |
| | + | + | + | + | + | + | + | | | |
| | + | + | + | + | + | + | | + | | |
| | + | + | + | + | + | + | | | + | |
| | + | + | + | + | + | + | | | | + |
| | + | + | + | + | + | | + | + | | |
| | + | + | + | + | + | | + | | + | |
| | + | + | + | + | + | | + | | | + |
| | + | + | + | + | + | | | + | + | |
| | + | + | + | + | + | | | + | | + |
| | + | + | + | + | + | | | | + | + |
| | + | + | + | + | | + | + | + | | |
| | + | + | + | + | | + | + | | + | |
| | + | + | + | + | | + | + | | | + |
| | + | + | + | + | | + | | + | + | |
| | + | + | + | + | | + | | + | | + |
| | + | + | + | + | | + | | | + | + |
| | + | + | + | + | | | + | + | + | |
| | + | + | + | + | | | + | + | | + |
| | + | + | + | + | | | + | | + | + |
| | + | + | + | + | | | | + | + | + |
| | + | + | + | | + | + | + | + | | |
| | + | + | + | | + | + | + | | + | |
| | + | + | + | | + | + | + | | | + |
| | + | + | + | | + | + | | + | + | |
| | + | + | + | | + | + | | + | | + |
| | + | + | + | | + | + | | | + | + |
| | + | + | + | | + | | + | + | + | + |
| | + | + | + | | | + | + | + | + | + |
| | + | + | | + | + | + | + | + | | |
| | + | + | | + | + | + | + | | + | |
| | + | + | | + | + | + | + | | | + |
| | + | + | | + | + | + | | + | + | |
| | + | + | | + | + | + | | + | | + |
| | + | + | | + | + | + | | | + | + |
| | + | + | | + | + | | + | + | + | |
| | + | + | | + | + | | + | + | | + |
| | + | + | | + | + | | + | | + | + |
| | + | + | | + | | + | + | + | + | |
| | + | + | | + | | + | + | + | | + |
| | + | + | | + | | + | + | | + | + |
| | + | + | | + | | + | | + | + | + |
| | + | + | | + | | | + | + | + | + |
| | + | + | | | + | + | + | + | + | |
| | + | + | | | + | + | + | + | | + |
| | + | + | | | + | + | + | | + | + |
| | + | + | | | + | + | | + | + | + |
| | + | + | | | + | | + | + | + | + |
| | + | + | | | | + | + | + | + | + |
| | + | | + | + | + | + | + | + | | |
| | + | | + | + | + | + | + | | + | |
| | + | | + | + | + | + | + | | | + |
| | + | | | + | + | + | + | + | + | |
| | + | | | + | + | + | + | + | | + |
| | + | | | | + | + | + | + | + | + |
| | | + | + | + | + | + | + | + | | |
| | | + | + | + | + | + | + | | + | |
| | | + | + | + | + | + | + | | | + |
| | | + | + | + | + | + | | + | + | |
| | | + | + | + | + | + | | + | | + |
| | | + | + | + | + | + | | | + | + |
| | | + | + | + | + | | + | + | + | |
| | | + | + | + | + | | + | + | | + |
| | | + | + | + | + | | + | | + | + |
| | | + | + | + | + | | | + | + | + |
| | | + | + | + | | + | + | + | + | |
| | | + | + | + | | + | + | + | | + |
| | | + | + | + | | + | + | | + | + |
| | | + | + | + | | + | | + | + | + |
| | | + | + | + | | | + | + | + | + |
| | | + | + | | + | + | + | + | + | |
| | | + | + | | + | + | + | + | | + |
| | | + | + | | + | + | + | | + | + |
| | | + | + | | + | + | | + | + | + |
| | | + | + | | + | | + | + | + | + |
| | | + | | + | + | + | + | + | + | |
| | | + | | + | + | + | + | + | | + |
| | | + | | + | + | + | + | | + | + |
| | | + | | + | + | + | | + | + | + |
| | | + | | + | + | | + | + | + | + |
| | | + | | + | | + | + | + | + | + |
| | | + | | | + | + | + | + | + | + |
| | | | + | + | + | + | + | + | + | |
| | | | + | + | + | + | + | + | | + |
| | | | + | + | + | + | + | | + | + |
| | | | + | + | + | + | | + | + | + |
| | | | + | + | + | | + | + | + | + |
| | | | + | + | | + | + | + | + | + |
| | | | | + | + | + | + | + | + | + |
| + | + | + | + | + | + | + | + | | | |
| + | + | + | + | + | + | + | | + | | |
| + | + | + | + | + | + | + | | | + | |
| + | + | + | + | + | + | + | | | | + |
| + | + | + | + | + | + | + | | + | + | |
| + | + | + | + | + | + | + | | + | | + |
| + | + | + | + | + | + | + | | | + | + |
| + | + | + | + | + | + | | + | + | | |
| + | + | + | + | + | + | | + | | + | |
| + | + | + | + | + | + | | + | | | + |
| + | + | + | + | + | + | | | + | + | |
| + | + | + | + | + | + | | | + | | + |
| + | + | + | + | + | + | | | | + | + |
| + | + | + | + | + | | + | + | + | | |
| + | + | + | + | + | | + | + | | + | |
| + | + | + | + | + | | + | + | | | + |
| + | + | + | + | + | | + | | + | + | |
| + | + | + | + | + | | + | | + | | + |
| + | + | + | + | + | | + | | | + | + |
| + | + | + | + | + | | | + | + | + | |
| + | + | + | + | + | | | + | + | | + |
| + | + | + | + | + | | | + | | + | + |
| + | + | + | + | + | | | | + | + | + |
| + | + | + | + | | + | + | + | + | | |
| + | + | + | + | | + | + | + | | + | |
| + | + | + | + | | + | + | + | | | + |
| + | + | + | + | | + | + | | + | + | |
| + | + | + | + | | + | + | | + | | + |
| + | + | + | + | | + | + | | | + | + |
| + | + | + | + | | + | | + | + | + | |
| + | + | + | + | | + | | + | + | | + |
| + | + | + | + | | + | | + | | + | + |
| + | + | + | + | | + | | | + | + | + |
| + | + | + | + | | | + | + | + | + | |
| + | + | + | + | | | + | + | + | | + |
| + | + | + | + | | | + | + | | + | + |
| + | + | + | + | | | + | | + | + | + |
| + | + | + | + | | | | + | + | + | + |
| + | + | + | | + | + | + | + | + | | |
| + | + | + | | + | + | + | + | | + | |
| + | + | + | | + | + | + | + | | | + |
| + | + | + | | + | + | + | | + | + | |
| + | + | + | | + | + | + | | + | | + |
| + | + | + | | + | + | + | | | + | + |
| + | + | + | | + | + | | + | + | + | |
| + | + | + | | + | + | | + | + | | + |
| + | + | + | | + | + | | + | | + | + |
| + | + | + | | + | + | | | + | + | + |
| + | + | + | | + | | + | + | + | + | |
| + | + | + | | + | | + | + | + | | + |
| + | + | + | | + | | + | + | | + | + |
| + | + | + | | + | | + | | + | + | + |
| + | + | + | | + | | | + | + | + | + |
| + | + | + | | | + | + | + | + | + | |
| + | + | + | | | + | + | + | + | | + |
| + | + | + | | | + | + | + | | + | + |
| + | + | + | | | + | + | | + | + | + |
| + | + | + | | | + | | + | + | + | + |
| + | + | + | | | | + | + | + | + | + |
| + | + | | + | + | + | + | + | + | | |
| + | + | | + | + | + | + | + | | + | |
| + | + | | + | + | + | + | + | | | + |
| + | + | | + | + | + | + | | + | + | |
| + | + | | + | + | + | + | | + | | + |
| + | + | | + | + | + | + | | | + | + |
| + | + | | + | + | + | | + | + | + | |
| + | + | | + | + | + | | + | + | | + |
| + | + | | + | + | + | | + | | + | + |
| + | + | | + | + | + | | | + | + | + |
| + | + | | + | + | | + | + | + | + | |
| + | + | | + | + | | + | + | + | | + |
| + | + | | + | + | | + | + | | + | + |
| + | + | | + | + | | | | + | + | + |
| + | + | | + | + | | | + | + | + | + |
| + | + | | + | | + | + | + | + | + | |
| + | + | | + | | + | + | + | + | | + |
| + | + | | + | | + | + | + | | + | + |
| + | + | | + | | + | + | | + | + | + |
| + | + | | + | | + | | + | + | + | + |
| + | + | | + | | | + | + | + | + | + |
| + | + | | | + | + | + | + | + | + | |
| + | + | | | + | + | + | + | + | | + |
| + | + | | | + | + | + | + | | + | + |
| + | + | | | + | + | + | | + | + | + |
| + | + | | | + | + | | + | + | + | + |
| + | + | | | | + | + | + | + | + | + |
| + | | + | + | + | + | + | + | + | | |
| + | | + | + | + | + | + | + | | + | |
| + | | + | + | + | + | + | + | | | + |
| + | | + | + | + | + | + | | + | + | |
| + | | + | + | + | + | + | | + | | + |
| + | | + | + | + | + | + | | | + | + |
| + | | + | + | + | + | | + | + | + | |
| + | | + | + | + | + | | + | + | | + |
| + | | + | + | + | + | | + | | + | + |
| + | | + | + | + | + | | | + | + | + |
| + | | + | + | + | | + | + | + | + | |
| + | | + | + | + | | + | + | + | | + |
| + | | + | + | + | | + | + | | + | + |
| + | | + | + | + | | + | | + | + | + |
| + | | + | + | + | | | + | + | + | + |
| + | | + | + | | + | + | + | + | + | |
| + | | + | + | | + | + | + | + | | + |
| + | | + | + | | + | + | + | | + | + |
| + | | + | + | | + | | + | + | + | + |
| + | | + | | + | + | + | + | + | + | |
| + | | + | | + | + | + | + | + | | + |
| + | | + | | + | + | + | + | | + | + |
| + | | + | | + | + | + | | + | + | + |
| + | | + | | + | | + | + | + | + | + |
| + | | + | | | + | + | + | + | + | + |
| + | | | + | + | + | + | + | + | + | |
| + | | | + | + | + | + | + | + | | + |
| + | | | + | + | + | + | + | | + | + |
| + | | | + | + | + | + | | + | + | + |
| + | | | + | + | + | | + | + | + | + |
| + | | | + | + | | + | + | + | + | + |
| + | | | + | | + | + | + | + | + | + |
| + | | | | + | + | + | + | + | + | + |
| | + | + | + | + | + | + | + | + | | |
| | + | + | + | + | + | + | + | | + | |
| | + | + | + | + | + | + | + | | | + |
| | + | + | + | + | + | + | | + | + | |
| | + | + | + | + | + | + | | + | | + |
| | + | + | + | + | + | + | | | + | + |
| | + | + | + | + | + | | + | + | + | |
| | + | + | + | + | + | | + | + | | + |
| | + | + | + | + | + | | | + | + | + |
| | + | + | + | + | | + | + | + | + | |
| | + | + | + | + | | + | + | + | | + |
| | + | + | + | + | | + | + | | + | + |
| | + | + | + | + | | + | | + | + | + |
| | + | + | + | + | | | + | + | + | + |
| | + | + | + | | + | + | + | + | + | |
| | + | + | + | | + | + | + | + | | + |
| | + | + | + | | + | + | + | | + | + |
| | + | + | + | | + | + | | + | + | + |
| | + | + | + | | + | | + | + | + | + |
| | + | + | + | | | + | + | + | + | + |
| | + | + | | + | + | + | + | + | + | |
| | + | + | | + | + | + | + | + | | + |
| | + | + | | + | + | + | + | | + | + |
| | + | + | | + | + | + | | + | + | + |
| | + | + | | + | + | | + | + | + | + |
| | + | + | | + | | + | + | + | + | + |
| | + | + | | | + | + | + | + | + | + |
| | + | | + | + | + | + | + | + | + | |
| | + | | + | + | + | + | + | + | | + |
| | + | | + | | + | + | + | + | + | + |
| | + | | + | + | | + | + | + | + | + |
| | + | | + | + | + | | + | + | + | + |
| | + | | + | + | + | + | | + | + | + |
| | + | | + | + | + | + | + | | + | + |
| | + | | + | + | + | + | + | + | | + |
| | + | | | + | + | + | + | + | + | + |
| | | + | + | + | + | + | + | + | + | |
| | | + | + | + | + | + | + | + | | + |
| | | + | + | + | + | + | + | | + | + |
| | | + | + | + | + | + | | + | + | + |
| | | + | + | + | + | | + | + | + | + |
| | | + | + | + | | + | + | + | + | + |
| | | + | + | | + | + | + | + | + | + |
| | | + | | + | + | + | + | + | + | + |
| | | | + | + | + | + | + | + | + | + |
| + | + | + | + | + | + | + | + | + | | |
| + | + | + | + | + | + | + | + | | + | |
| + | + | + | + | + | + | + | + | | | + |
| + | + | + | + | + | + | + | | + | + | |
| + | + | + | + | + | + | + | | + | | + |
| + | + | + | + | + | + | + | | | + | + |
| + | + | + | + | + | + | | + | + | + | |
| + | + | + | + | + | + | | + | + | | + |
| + | + | + | + | + | + | | + | | + | + |
| + | + | + | + | + | + | | | + | + | + |
| + | + | + | + | + | | + | + | + | + | |
| + | + | + | + | + | | + | + | + | | + |
| + | + | + | + | + | | + | + | | + | + |
| + | + | + | + | + | | + | | + | + | + |
| + | + | + | + | | + | + | + | + | + | |
| + | + | + | + | | + | + | + | + | | + |
| + | + | + | + | | + | + | + | | + | + |
| + | + | + | + | | + | + | | + | + | + |
| + | + | + | + | | + | | + | + | + | + |
| + | + | + | | + | + | + | + | + | + | |
| + | + | + | | + | + | + | + | + | | + |
| + | + | + | | + | + | + | + | | + | + |
| + | + | + | | + | + | + | | + | + | + |
| + | + | + | | + | + | | + | + | + | + |
| + | + | + | | + | | + | + | + | + | + |
| + | + | + | | | + | + | + | + | + | + |
| + | + | | + | + | + | + | + | + | + | |
| + | + | | + | + | + | + | + | + | | + |
| + | + | | + | + | + | + | + | | + | + |
| + | + | | + | + | + | + | | + | + | + |
| + | + | | + | + | + | | + | + | + | + |
| + | + | | + | + | | + | + | + | + | + |
| + | + | | + | | + | + | + | + | + | + |
| + | + | | | + | + | + | + | + | + | + |
| + | | + | + | + | + | + | + | + | + | |
| + | | + | + | + | + | + | + | + | | + |
| + | | + | + | + | + | + | + | | + | + |
| + | | + | + | + | + | + | | + | + | + |
| + | | + | + | + | + | | + | + | + | + |
| + | | + | + | + | | + | + | + | + | + |
| + | | + | + | | + | + | + | + | + | + |
| + | | + | | + | + | + | + | + | + | + |
| + | | | + | + | + | + | + | + | + | + |
| | + | + | + | + | + | + | + | + | + | |
| | + | + | + | + | + | + | + | + | | + |
| | + | + | + | + | + | + | + | | + | + |
| | + | + | + | + | + | + | | + | + | + |
| | + | + | + | + | + | | + | + | + | + |
| | + | + | + | + | | + | + | + | + | + |
| | + | + | + | | + | + | + | + | + | + |
| | + | + | | + | + | + | + | + | + | + |
| | + | | + | + | + | + | + | + | + | + |
| | | + | + | + | + | + | + | + | + | + |
| + | + | + | + | + | + | + | + | + | + | |
| + | + | + | + | + | + | + | + | + | | + |
| + | + | + | + | + | + | + | + | | + | + |
| + | + | + | + | + | + | + | | + | + | + |
| + | + | + | + | + | + | | + | + | + | + |
| + | + | + | + | + | | + | + | + | + | + |
| + | + | + | + | | + | + | + | + | + | + |
| + | + | + | | + | + | + | + | + | + | + |
| + | + | | + | + | + | + | + | + | + | + |
| + | | + | + | + | + | + | + | + | + | + |
| | + | + | + | + | + | + | + | + | + | + |
| + | + | + | + | + | + | + | + | + | + | + |

In one embodiment of the present invention, the compound according to formula I is provided wherein R1 is deuterium and R2, R3, R4, R5, R6, R7, R8, R9, R10 and R11 are selected from deuterium and hydrogen, for example the compound according to formula I wherein R1 is deuterium and R2, R3, R4, R5, R6, R7, R8, R9, R10 and R11 are hydrogen, or for example the compound according to formula I wherein R2 is deuterium and R1, R3, R4, R5, R6, R7, R8, R9, R10 and R11 are hydrogen, or for example the compound according to formula I wherein R3 is deuterium and R1, R2, R4, R5, R6, R7, R8, R9, R10 and R11 are hydrogen, or for example the compound according to formula I wherein R4 is deuterium and R1, R2, R3, R5, R6, R7, R8, R9, R10 and R11 are hydrogen, or for example the compound according to formula I wherein R5 is deuterium and R1, R2, R3, R4, R6, R7, R8, R9, R10 and R11 are hydrogen, or for example the compound according to formula I wherein R6 is deuterium and R1, R2, R3, R4, R5, R7, R8, R9, R10 and R11 are hydrogen, or for example the compound according to formula I wherein R6 is deuterium and R1, R2, R3, R4, R5, R7, R8, R9, R10 and R11 are hydrogen, or for example the compound according to formula I wherein R7 is deuterium and R1, R2, R3, R4, R5, R6, R8, R9, R10 and R11 are hydrogen, or for example the compound according to formula I wherein R8 is deuterium and R1, R2, R3, R4, R5, R6, R7, R9, R10 and R11 are hydrogen, or for example the compound according to formula I wherein R9 is deuterium and R1, R2, R3, R4, R5, R6, R7, R8, R10 and R11 are hydrogen, or for example the compound according to formula I wherein R9 is deuterium and R1, R2, R3, R4, R5, R6, R7, R8, R10 and R11 are hydrogen, or for example the compound according to formula I wherein R10 is deuterium and R1, R2, R3, R4, R5, R6, R7, R8, R9 and R11 are hydrogen, or for example the compound according to formula I wherein R11 is deuterium and R1, R2, R3, R4, R5, R6, R7, R8, R9 and R10 are hydrogen.

In another embodiment of the present invention, the compound according to formula I is provided wherein R1 is deuterium and one or more of R2, R3, R4, R5, R6, R7, R8, R9, R10, and R11 is deuterium, or the compound of formula I wherein R2 is deuterium and one or more of R1, R3, R4, R5, R6, R7, R8, R9, R10, and R11 is deuterium, or the compound of formula I wherein R3 is deuterium and one or more of R1, R2, R4, R5, R6, R7, R8, R9, R10, and R11 is deuterium, or the compound of formula I wherein R4 is deuterium and one or more of R1, R2, R3, R5, R6, R7, R8, R9, R10, and R11 is deuterium, or the compound of formula I wherein R5 is deuterium and one or more of R1, R2, R3, R4, R6, R7, R8, R9, R10, and R11 is deuterium, or the compound of formula I wherein R6 is deuterium and one or more of R1, R2, R3, R4, R5, R7, R8, R9, R10, and R11 is deuterium, or the compound of formula I wherein R7 is deuterium and one or more of R1, R2, R3, R4, R5, R6, R8, R9, R10, and R11 is deuterium, or the compound of formula I wherein R8 is deuterium and one or more of R1, R2, R3, R4, R5, R6, R7, R9, R10, and R11 is deuterium, or the compound of formula I wherein R9 is deuterium and one or more of R1, R2, R3, R4, R5, R6, R7, R8, R10, and R11 is deuterium, or the compound of formula I wherein R10 is deuterium and one or more of R1, R2, R3, R4, R5, R6, R7, R8, R9, and R11 is deuterium, or the compound of formula I wherein R11 is deuterium and one or more of R1, R2, R3, R4, R5, R6, R7, R8, R9, and R10 is deuterium.

In yet another embodiment of the present invention, the compound according to formula I is provided wherein all positions of R1, R2, R3, R4, R5, R6, R7, R8, R9, R10, and R11 are deuterium.

In one embodiment of the present invention, the compound according to formula I is provided, wherein R1, R2, R3, R10 and R11 are individually selected from the group consisting of hydrogen (H) and deuterium (D) with the proviso that at least one of R1, R2, R3, R10 and R11 is deuterium.

In one embodiment of the present invention, the compound according to formula I is provided wherein R1 is deuterium and R2, R3, R10 and R11 are selected from deuterium and hydrogen, for example the compound according to formula I wherein R1 is deuterium and R2, R3, R10 and R11 are hydrogen, or for example the compound according to formula I wherein R2 is deuterium and R1, R3, R10 and R11 are hydrogen, or for example the compound according to formula I wherein R3 is deuterium and R1, R2, R10 and R11 are hydrogen, or for example the compound according to formula I wherein R10 is deuterium and R1, R2, R3 and R11 are hydrogen, or for example the compound according to formula I wherein R11 is deuterium and R1, R2, R3 and R10 are hydrogen.

In another embodiment of the present invention, the compound according to formula I is provided wherein two positions of R1, R2, R3, R10 and R11 are deuterium and the others are hydrogen. This embodiment includes for example the compound according to formula I wherein R1 and R2 are deuterium and R3, R10 and R11 are hydrogen, or for example the compound according to formula I wherein R1 and R3 are deuterium and R2, R10 and R11 are hydrogen, or for example the compound according to formula I wherein R1 and R10 are deuterium and R2, R3 and R11 are hydrogen, or for example the compound according to formula I wherein R1 and R11 are deuterium and R2, R3 and R10, or for example the compound according to formula I wherein R2 and R3 are deuterium and R1, R10 and R11 are hydrogen, or for example the compound according to formula I wherein R2 and R10 are deuterium and R1, R3 and R11, or for example the compound according to formula I wherein R2 and R11 are deuterium and R1, R3 and R10 are hydrogen, or for example the compound according to formula I wherein R3 and R10 are deuterium and R1, R2 and R11 are hydrogen, or for example the compound according to formula I wherein R3 and R11 are deuterium and R1, R2 and R10 are hydrogen, or for example the compound according to formula I wherein R10 and R11 are deuterium and R1, R2 and R3 are hydrogen.

In yet another embodiment of the present invention, the compound according to formula I is provided wherein three positions of R1, R2, R3, R10 and R11 are deuterium and the others are hydrogen. This embodiment includes for example the compound according to formula I wherein R1, R2 and R3 are deuterium and R10 and R11 are hydrogen, or for example the compound according to formula I wherein R1, R2 and R10 are deuterium and R3 and R11 are hydrogen, or for example the compound according to formula I wherein R1, R2 and R11 are deuterium and R3 and R10 are hydrogen, or for example the compound according to formula I wherein R1, R3 and R10 are deuterium and R2 and R11 are hydrogen, or for example the compound according to formula I wherein R1, R10 and R11 are deuterium and R2 and R3 are hydrogen, or for example the compound according to formula I wherein R2, R3 and R10 are deuterium and R1 and R11 are hydrogen, or for example the compound according to formula I wherein R2, R10 and R11 are deuterium and R1 and R3 are hydrogen, or for example the compound according to formula I wherein R3, R10 and R11 are deuterium and R1 and R2 are hydrogen.

In yet another embodiment of the present invention, the compound according to formula I is provided wherein four positions of R1, R2, R3, R10 and R11 are deuterium and the others are hydrogen. This embodiment includes for example the compound according to formula I wherein R1, R2, R3 and R10 are deuterium and R11 is hydrogen, or for example the compound according to formula I wherein R1, R2, R3 and R11 are deuterium and R10 is hydrogen, or for example the compound according to formula I wherein R1, R2, R10 and R11 are deuterium and R3 is hydrogen, or for example the compound according to formula I wherein R1, R3, R10 and R11 are deuterium and R2 is hydrogen, or for example the compound according to formula I wherein R2, R3, R10 and R11 are deuterium and R1 is hydrogen.

In yet another embodiment of the present invention, the compound according to formula I is provided wherein all positions of R1, R2, R3, R10 and R11 are deuterium.

In one embodiment of the present invention, the compound according to formula I is provided wherein R1 is deuterium and R2, R3, R and R5 are selected from deuterium and hydrogen, for example the compound according to formula I wherein R1 is deuterium and R2, R3, R4 and R5 are hydrogen, or for example the compound according to formula I wherein R2 is deuterium and R1, R3, R4 and R5 are hydrogen, or for example the compound according to formula I wherein R3 is deuterium and R1, R2, R4 and R5 are hydrogen, or for example the compound according to formula I wherein R4 is deuterium and R1, R2, R3 and R5 are hydrogen, or for example the compound according to formula I wherein R5 is deuterium and R1, R2, R3 and R4 are hydrogen.

In another embodiment of the present invention, the compound according to formula I is provided wherein two positions of R1, R2, R3, R4 and R5 are deuterium and the others are hydrogen. This embodiment includes for example the compound according to formula I wherein R1 and R2 are deuterium and R3, R4 and R5 are hydrogen, or for example the compound according to formula I wherein R1 and R3 are deuterium and R2, R4 and R5 are hydrogen, or for example the compound according to formula I wherein R1 and R4 are deuterium and R2, R3 and R5 are hydrogen, or for example the compound according to formula I wherein R1 and R5 are deuterium and R2, R3 and R4, or for example the compound according to formula I wherein R2 and R3 are deuterium and R1, R4 and R5 are hydrogen, or for example the compound according to formula I wherein R2 and R4 are deuterium and R1, R3 and R5, or for example the compound according to formula I wherein R2 and R5 are deuterium and R1, R3 and R4 are hydrogen, or for example the compound according to formula I wherein R3 and R4 are deuterium and R1, R2 and R5 are hydrogen, or for example the compound according to formula I wherein R3 and R5 are deuterium and R1, R2 and R4 are hydrogen, or for example the compound according to formula I wherein R4 and R5 are are deuterium and R1, R2 and R3 are hydrogen.

In yet another embodiment of the present invention, the compound according to formula I is provided wherein three positions of R1, R2, R3, R4 and R5 are deuterium and the others are hydrogen. This embodiment includes for example the compound according to formula I wherein R1, R2 and R3 are deuterium and R4 and R5 are hydrogen, or for example the compound according to formula I wherein R1, R2 and R4 are deuterium and R3 and R5 are hydrogen, or for example the compound according to formula I wherein R1, R2 and R5 are deuterium and R3 and R4 are hydrogen, or for example the compound according to formula I wherein R1, R3 and R4 are deuterium and R2 and R5 are hydrogen, or for example the compound according to formula I wherein R1, R4 and R5 are deuterium and R2 and R3 are hydrogen, or for example the compound according to formula I wherein R2, R3 and R4 are deuterium and R1 and R5 are hydrogen, or for example the compound according to formula I wherein R2, R4 and R5 are deuterium and R1 and R3 are hydrogen, or for example the compound according to formula I wherein R3, R4 and R5 are deuterium and R1 and R2 are hydrogen.

In yet another embodiment of the present invention, the compound according to formula I is provided wherein four positions of R1, R2, R3, R4 and R5 are deuterium and the others are hydrogen. This embodiment includes for example the compound according to formula I wherein R1, R2, R3 and R4 are deuterium and R5 is hydrogen, or for example the compound according to formula I wherein R1, R2, R3 and R5 are deuterium and R4 is hydrogen, or for example the compound according to formula I wherein R1, R2, R4 and R5 are deuterium and R3 is hydrogen, or for example the compound according to formula I wherein R1, R3, R4 and R5 are deuterium and R2 is hydrogen, or for example the compound according to formula I wherein R2, R3, R4 and R5 are deuterium and R1 is hydrogen.

In yet another embodiment of the present invention, the compound according to formula I is provided wherein all positions of R1, R2, R3, R4 and R5 are deuterium.

It is understood that the present invention also relates to chiral analogues of deuterated tandospirone, such as enantiomers or optical isomers of the compounds and pharmaceutical compositions of the present invention. Such enantiomers or optical isomers may have different pharmacokinetic properties and therefore different effects during treatment.

In another embodiment, the compound is selected from the group of (1R,2R,6S,7S)-4-{4-[4-((5-²H)pyrimidin-2-yl)piperazin-1-yl]butyl}-4-azatricyclo[5.2.1.0^{2,6}]decane-3,5-dione, (1R,2R,6S,7S)-4-{4-[4-(pyrimidin-2-yl)piperazin-1-yl]-4-(²H₂)butyl}-4-azatricyclo[5.2.1.0^{2,6}]decane-3,5-dione, (1R,2R,6S,7S)-4-{4-[4-((5-²H)pyrimidin-2-yl)piperazin-1-yl]-(4-²H₂)butyl}-4-azatricyclo[5.2.1.0^{2,6}]decane-3,5-dione, (1R,2R,6S,7S)-4-{4-[4-(pyrimidin-2-yl)piperazin-1-yl]butyl}-(8,9-²H₂)-4-azatricyclo[5.2.1.0^{2,6}]decane-3,5-dione, (1R,2R,6S,7S)-4-{4-[4-((5-²H)pyrimidin-2-yl)piperazin-1-yl]butyl}-(8,9-²H₂)-4-azatricyclo[5.2.1.0^{2,6}]decane-3,5-dione, (1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione, (1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione, (1R,2R,6S,7S)-4-{4-[4-(pyrimidin-2-yl)piperazin-1-yl]-4-(²H)butyl}-4-azatricyclo[5.2.1.0^{2,6}]decane-3,5-dione, (1R,2R,6S,7S)-4-{4-[4-((5-²H)pyrimidin-2-yl)piperazin-1-yl]-(4-²H)butyl}-4-azatricyclo[5.2.1.0^{2,6}]decane-3,5-dione, (1R,2R,6S,7S)-4-{4-[4-(pyrimidin-2-yl)piperazin-1-yl]butyl}-(8-²H)-4-azatricyclo[5.2.1.0^{2,6}]decane-3,5-dione, (1R,2R,6S,7S)-4-{4-[4-((5-²H)pyrimidin-2-yl)piperazin-1-yl]butyl}-(8-²H₂)-4-azatricyclo[5.2.1.0^{2,6}]decane-3,5-dione, (1R,2R,6S,7S)-4-{4-[4-(pyrimidin-2-yl)piperazin-1-yl]-(4-²H)butyl}-(8-²H₂)-4-azatricyclo[5.2.1.0^{2,6}]decane-3,5-dione, (1R,2R,6S,7S)-4-{4-[4-((5-²H)pyrimidin-2-yl)piperazin-1-yl]-(4-²H)butyl}-(8-²H₂)-4-azatricyclo[5.2.1.0^{2,6}]decane-3,5-dione.

In one embodiment of the present invention, the compounds are selected from the list consisting of (1R,2R,6S,7S)-4-{4-[4-((5-²H)pyrimidin-2-yl)piperazin-1-yl]butyl}-4-azatricyclo[5.2.1.0^{2,6}]decane-3,5-dione, (1R,2R,6S,7S)-4-{4-[4-(pyrimidin-2-yl)piperazin-1-yl]-4-(²H₂)butyl}-4-azatricyclo[5.2.1.0^{2,6}]decane-3,5-dione,
(1R,2R,6S,7S)-4-{4-[4-((5-²H)pyrimidin-2-yl)piperazin-1-yl]-(4-²H₂)butyl}-4-azatricyclo[5.2.1.0^{2,6}]decane-3,5-dione, (1R,2R,6S,7S)-4-{4-[4-(pyrimidin-2-yl)piperazin-1-yl]butyl}-(8,9-²H₂)-4-azatricyclo[5.2.1.0^{2,6}]decane-3,5-dione,
(1R,2R,6S,7S)-4-{4-[4-((5-²H)pyrimidin-2-yl)piperazin-1-yl]butyl}-(8,9-²H₂)-4-azatricyclo[5.2.1.0^{2,6}]decane-3,5-dione, (1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione and (1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione.

In a preferred embodiment of the present invention the compound has one or more deuterium in the positions R1, R2, R3, R4, R5, R6, R7, R10, R11, preferably such as the compounds presented in Tables II, III and IV consisting of: (1R,2R,6S,7S)-4-{4-[4-((5-²H)pyrimidin-2-yl)piperazin-1-yl]butyl}-4-azatricyclo[5.2.1.0^{2,6}]decane-3,5-dione (I), (1R,2R,6S,7S)-4-{4-[4-(pyrimidin-2-yl)piperazin-1-yl]-(4,4-²H₂)butyl}-4-azatricyclo[5.2.1.0^{2,6}]decane-3,5-dione (II), (1R,2R,6S,7S)-4-{4-[4-((5-²H)pyrimidin-2-yl)piperazin-1-yl]-(4,4-²H₂)butyl}-4-azatricyclo[5.2.1.0^{2,6}]decane-3,5-dione (III), (1R,2R,6S,7S)-4-{4-[4-(pyrimidin-2-yl)piperazin-1-yl]butyl}-(8,9-²H₂)-4-azatricyclo[5.2.1.0^{2,6}]decane-3,5-dione (IV), (1R,2R,6S,7S)-4-{4-[4-((5-²H)pyrimidin-2-yl)piperazin-1-yl]butyl}-(8,9-²H₂)-4-azatricyclo[5.2.1.0^{2,6}]decane-3,5-dione (V), (1R,2R,6S,7S)-4-{4-[4-(pyrimidin-2-yl)piperazin-1-yl]-(4,4-²H₂)butyl}-(8,9-²H₂)-4-azatricyclo[5.2.1.0^{2,6}]decane-3,5-dione (VI), (1R,2R,6S,7S)-4-{4-[4-((5-²H)pyrimidin-2-yl)piperazin-1-yl]-(4,4-²H₂)butyl}-(8,9-²H₂)-4-azatricyclo[5.2.1.0^{2,6}]decane-3,5-dione (VII), (1R,2R,6S,7S)-4-{4-[4-(pyrimidin-2-yl)piperazin-1-yl]-4-(²H)butyl}-4-azatricyclo[5.2.1.0^{2,6}]decane-3,5-dione (VIII), (1R,2R,6S,7S)-4-{4-[4-((5-²H)pyrimidin-2-yl)piperazin-1-yl]-(4-²H)butyl}-4-azatricyclo[5.2.1.0^{2,6}]decane-3,5-dione (IX), (1R,2R,6S,7S)-4-{4-[4-(pyrimidin-2-yl)piperazin-1-yl]butyl}-(8-²H)-4-azatricyclo[5.2.1.0^{2,6}]decane-3,5-dione (X), (1R,2R,6S,7S)-4-{4-[4-((5-²H)pyrimidin-2-yl)piperazin-1-yl]butyl}-(8-²H)-4-azatricyclo[5.2.1.0^{2,6}]decane-3,5-dione (XI), (1R,2R,6S,7S)-4-{4-[4-(pyrimidin-2-yl)piperazin-1-yl]-(4-²H)butyl}-(8-²H)-4-azatricyclo[5.2.1.0^{2,6}]decane-3,5-dione (XII), (1R,2R,6S,7S)-4-{4-[4-((5-²H)pyrimidin-2-yl)piperazin-1-yl]-(4-²H)butyl}-(8-²H)-4-azatricyclo[5.2.1.0^{2,6}]decane-3,5-dione (XIII), (1R,2R,6S,7S)-4-{4-[4-(pyrimidin-2-yl)piperazin-1-yl]butyl}-(2,6-²H₂)-4-azatricyclo[5.2.1.0^{2,6}]decane-3,5-dione (XIV), (1R,2R,6S,7S)-4-{4-[4-(pyrimidin-2-yl)piperazin-1-yl]-(4-²H₂)butyl}-(2,6-²H₂)-4-azatricyclo[5.2.1.0^{2,6}]decane-3,5-dione (XV), (1R,2R,6S,7S)-4-{4-[4-((5-²H)pyrimidin-2-yl)piperazin-1-yl]-(4,4-²H₂)butyl}-(2,6-²H₂)-4-azatricyclo[5.2.1.0^{2,6}]decane-3,5-dione (XVI), (1R,2R,6S,7S)-4-{4-[4-((5-²H)pyrimidin-2-yl)piperazin-1-yl]butyl}-(2,6-²H₂)-4-azatricyclo[5.2.1.0^{2,6}]decane-3,5-dione (XVII), (1R,2R,6S,7S)-4-{4-[4-(pyrimidin-2-yl)piperazin-1-yl]-(2,2,3,3,4,4-²H₆)butyl}-4-azatricyclo[5.2.1.0^{2,6}]decane-3,5-dione (XVIII), (1R,2R,6S,7S)-4-{4-[4-(pyrimidin-2-yl)piperazin-1-yl]-(2,2,3,3,4,4-²H₆)butyl}-(8,9-²H₂)-4-azatricyclo[5.2.1.0^{2,6}]decane-3,5-dione (XIX), (1R,2R,6S,7S)-4-{4-[4-((5-²H)pyrimidin-2-yl)piperazin-1-yl]-(2,2,3,3,4,4-²H₆)butyl}-4-azatricyclo[5.2.1.0^{2,6}]decane-3,5-dione (XX,), (1R,2R,6S,7S)-4-{4-[4-((5-²H)pyrimidin-2-yl)piperazin-1-yl]-(2,2,3,3,4,4-²H6)butyl}-(8,9-²H₂)-4-azatricyclo[5.2.1.0^{2,6}]decane-3,5-dione (XXI), (1R,2R,6S,7S)-4-{4-[4-((pyrimidin-2-yl)piperazin-1-yl]butyl}-(2,6,8,9-²H4)-4-azatricyclo[5.2.1.0^{2,6}]decane-3,5-dione (XXII), (1R,2R,6S,7S)-4-{4-[4-((pyrimidin-2-yl)piperazin-1-yl]-(4,4-²H₂)butyl}-(2,6,8,9-²H4)-4-azatricyclo[5.2.1.0^{2,6}]decane-3,5-dione (XXIII), (1R,2R,6S,7S)-4-{4-[4-((pyrimidin-2-yl)piperazin-1-yl]-(4,4-²H₂)butyl}-(2,6-²H₂)-4-azatricyclo[5.2.1.0^{2,6}]decane-3,5-dione (XXIV), (1R,2R,6S,7S)-4-{4-[4-((pyrimidin-2-yl)piperazin-1-yl]-(2,2,3,3,4,4-²H₆)butyl}-(2,6-²H₂)-4-azatricyclo[5.2.1.0^{2,6}]decane-3,5-dione (XXV), (1R,2R,6S,7S)-4-{4-[4-(pyrimidin-2-yl)piperazin-1-yl]-(2,2,3,3,4,4-²H₆)butyl}-(2,6,8,9-²H₄)-4-azatricyclo[5.2.1.0^{2,6}]decane-3,5-dione (XXVI), (1R,2R,6S,7S)-4-{4-[4-((5-²H)pyrimidin-2-yl)piperazin-1-yl]-(2,2,3,3,4,4-²H₆)butyl}-(2,6,8,9-²H₄)-4-azatricyclo[5.2.1.0^{2,6}]decane-3,5-dione (XXVI).

TABLE II

| | |
|---|---|
| 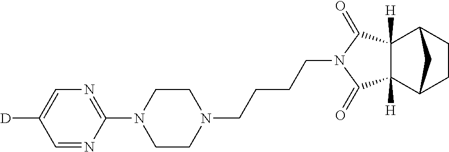 [SMILES: [2H]c1cnc(N2CCN(CCCCN3C(=O)[C@@H]4[C@@H]5CC[C@@H](C5)[C@@H]4C3=O)CC2)nc1] | I |
| 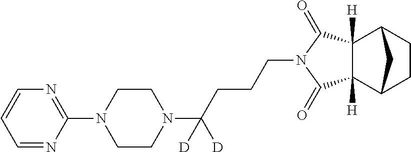 [SMILES: [2H]C([2H])(CCCN1C(=O)[C@@H]2[C@@H]3CC[C@@H](C3)[C@@H]2C1=O)N1CCN(c2ncccn2)CC1] | II |
| 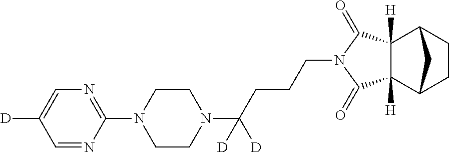 [SMILES: [2H]c1cnc(N2CCN(C([2H])([2H])CCCN3C(=O)[C@@H]4[C@@H]5CC[C@@H](C5)[C@@H]4C3=O)CC2)nc1] | III |
| 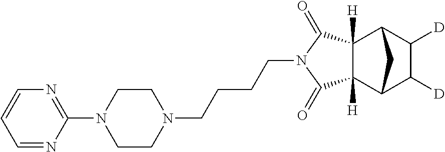 [SMILES: [2H]C1C([2H])[C@@H]2C[C@H]1[C@@H]1C(=O)N(CCCCN3CCN(c4ncccn4)CC3)C(=O)[C@@H]12] | IV |
| 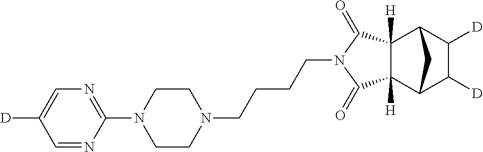 [SMILES: [2H]c1cnc(N2CCN(CCCCN3C(=O)[C@@H]4[C@H](C3=O)[C@H]3C[C@@H]4C([2H])C3[2H])CC2)nc1] | V |
| 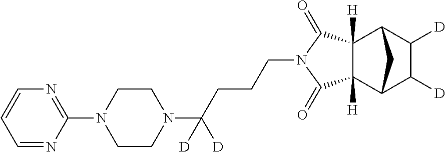 [SMILES: [2H]C1C([2H])[C@@H]2C[C@H]1[C@@H]1C(=O)N(CCCC([2H])([2H])N3CCN(c4ncccn4)CC3)C(=O)[C@@H]12] | VI |
| 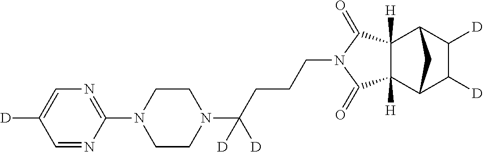 [SMILES: [2H]c1cnc(N2CCN(C([2H])([2H])CCCN3C(=O)[C@@H]4[C@H](C3=O)[C@H]3C[C@@H]4C([2H])C3[2H])CC2)nc1] | VII |
| 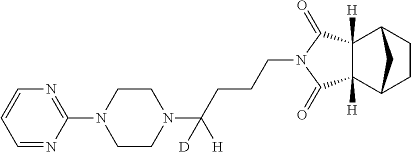 [SMILES: [2H]C(CCCN1C(=O)[C@@H]2[C@@H]3CC[C@@H](C3)[C@@H]2C1=O)N1CCN(c2ncccn2)CC1] | VIII |
| 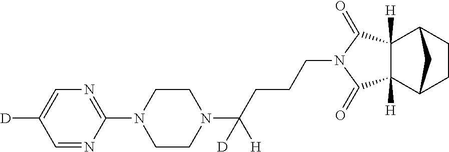 [SMILES: [2H]c1cnc(N2CCN(C([2H])CCCN3C(=O)[C@@H]4[C@@H]5CC[C@@H](C5)[C@@H]4C3=O)CC2)nc1] | IX |
| 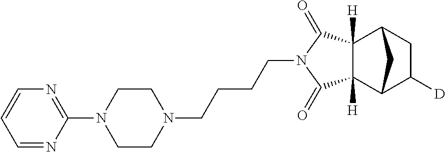 [SMILES: [2H]C1C[C@@H]2C[C@H]1[C@@H]1C(=O)N(CCCCN3CCN(c4ncccn4)CC3)C(=O)[C@H]21] | X |
| 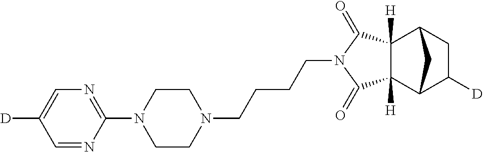 [SMILES: [2H]c1cnc(N2CCN(CCCCN3C(=O)[C@@H]4[C@@H]5CC([2H])[C@@H](C5)[C@@H]4C3=O)CC2)nc1] | XI |
| 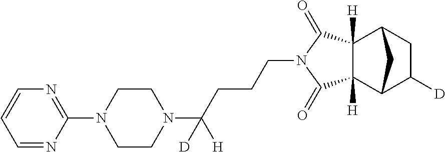 [SMILES: [2H]C1C[C@@H]2C[C@H]1[C@@H]1C(=O)N(CCCC([2H])N3CCN(c4ncccn4)CC3)C(=O)[C@H]21] | XII |
| 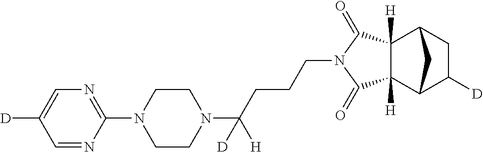 [SMILES: [2H]c1cnc(N2CCN(C([2H])CCCN3C(=O)[C@@H]4[C@@H]5CC([2H])[C@@H](C5)[C@@H]4C3=O)CC2)nc1] | XIII |

TABLE III

| | |
|---|---|
| 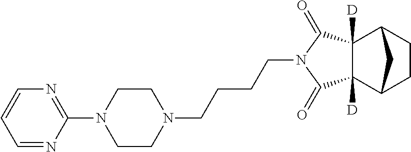 [SMILES: [2H][C@@]12C(=O)N(CCCCN3CCN(c4ncccn4)CC3)C(=O)[C@]1([2H])[C@@H]1CC[C@H]2C1] | XIV |
| 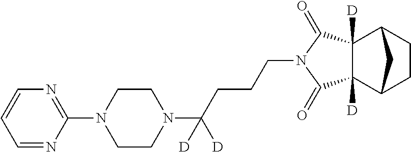 [SMILES: [2H]C([2H])(CCCN1C(=O)[C@@]2([2H])[C@H]3CC[C@H](C3)[C@@]2([2H])C1=O)N1CCN(c2ncccn2)CC1] | XV |
| 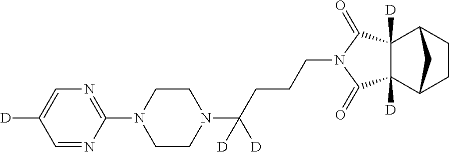 [SMILES: [2H]c1cnc(N2CCN(C([2H])([2H])CCCN3C(=O)[C@@]4([2H])[C@H]5CC[C@H](C5)[C@@]4([2H])C3=O)CC2)nc1] | XVI |
| 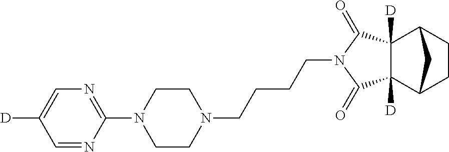 [SMILES: [2H]c1cnc(N2CCN(CCCCN3C(=O)[C@@]4([2H])[C@H]5CC[C@H](C5)[C@@]4([2H])C3=O)CC2)nc1] | XVII |
| 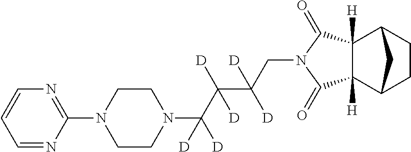 [SMILES: [2H]C([2H])(CN1C(=O)[C@@H]2[C@@H]3CC[C@@H](C3)[C@@H]2C1=O)C([2H])([2H])C([2H])([2H])N1CCN(c2ncccn2)CC1] | XVIII |
| 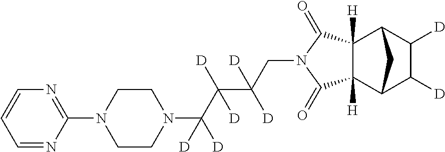 [SMILES: [2H]C1C([2H])[C@@H]2C[C@H]1[C@@H]1C(=O)N(CC([2H])([2H])C([2H])([2H])C([2H])([2H])N3CCN(c4ncccn4)CC3)C(=O)[C@@H]12] | XIX |
| 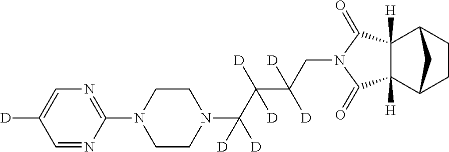 [SMILES: [2H]c1cnc(N2CCN(C([2H])([2H])C([2H])([2H])C([2H])([2H])CN3C(=O)[C@@H]4[C@@H]5CC[C@@H](C5)[C@@H]4C3=O)CC2)nc1] | XX |
| 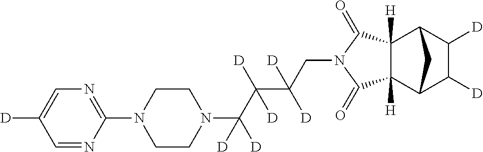 [SMILES: [2H]c1cnc(N2CCN(C([2H])([2H])C([2H])([2H])C([2H])([2H])CN3C(=O)[C@@H]4[C@H](C3=O)[C@H]3C[C@@H]4C([2H])C3[2H])CC2)nc1] | XXI |

TABLE IV

| | |
|---|---|
| 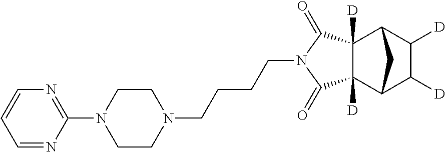 [SMILES: [2H]C1C([2H])[C@@H]2C[C@H]1[C@]1([2H])C(=O)N(CCCCN3CCN(c4ncccn4)CC3)C(=O)[C@]21[2H]] | XXII |
| 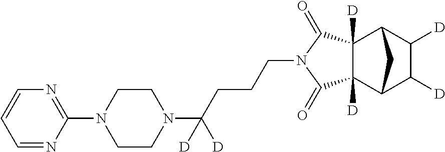 [SMILES: [2H]C1C([2H])[C@@H]2C[C@H]1[C@]1([2H])C(=O)N(CCCC([2H])([2H])N3CCN(c4ncccn4)CC3)C(=O)[C@]21[2H]] | XXIII |
| 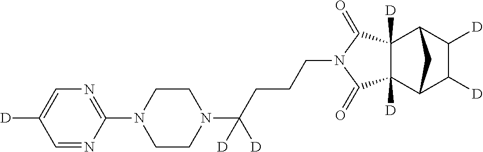 [SMILES: [2H]c1cnc(N2CCN(C([2H])([2H])CCCN3C(=O)[C@@]4([2H])[C@@H]5C[C@@H](C([2H])C5[2H])[C@@]4([2H])C3=O)CC2)nc1] | XXIV |
| 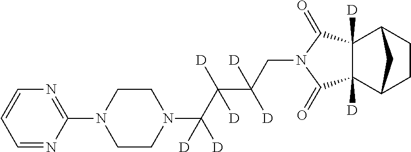 [SMILES: [2H]C([2H])(CN1C(=O)[C@@]2([2H])[C@H]3CC[C@H](C3)[C@@]2([2H])C1=O)C([2H])([2H])C([2H])([2H])N1CCN(c2ncccn2)CC1] | XXV |
| 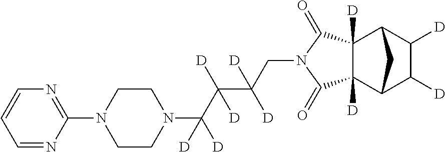 [SMILES: [2H]C1C([2H])[C@@H]2C[C@H]1[C@]1([2H])C(=O)N(CC([2H])([2H])C([2H])([2H])C([2H])([2H])N3CCN(c4ncccn4)CC3)C(=O)[C@]21[2H]] | XXVI |
| 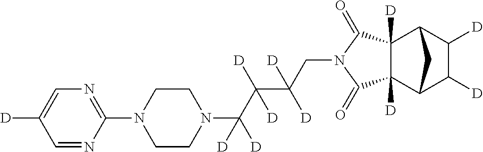 [SMILES: [2H]c1cnc(N2CCN(C([2H])([2H])C([2H])([2H])C([2H])([2H])CN3C(=O)[C@@]4([2H])[C@@H]5C[C@@H](C([2H])C5[2H])[C@@]4([2H])C3=O)CC2)nc1] | XXVII |

In an even more preferred embodiment of the present invention, the compounds according to formula I are selected from the group of compounds wherein one or both of the positions R2 and R3 are deuterium, and the positions R1, R4, R5, R6, R7, R8, R9, R10 and R11 are individually selected from deuterium or hydrogen.

In still an even more preferred embodiment of the present invention, the deuterated tandospirone compounds are selected from compounds having deuterium in both of the positions R2 and R3 selected from the group of (1R,2R,6S,7S)-4-{4-[4-(pyrimidin-2-yl)piperazin-1-yl]-(4,4-²H₂)butyl}-4-azatricyclo[5.2.1.0^{2,6}]decane-3,5-dione (II), (1R,2R,6S,7S)-4-{4-[4-((5-²H)pyrimidin-2-yl)piperazin-1-yl]-(4,4-²H₂)butyl}-4-azatricyclo[5.2.1.0^{2,6}]decane-3,5-dione (III), (1R,2R,6S,7S)-4-{4-[4-(pyrimidin-2-yl)piperazin-1-yl]-(4,4-²H₂)butyl}-(8,9-²H₂)-4-azatricyclo[5.2.1.0^{2,6}]decane-3,5-dione (VI), (1R,2R,6S,7S)-4-{4-[4-(((5-²H)pyrimidin-2-yl)piperazin-1-yl]-(4,4-²H₂)butyl}-(2,6-²H₂)-4-azatricyclo[5.2.1.0^{2,6}]decane-3,5-dione (VII), (1R,2R,6S,7S)-4-{4-[4-(pyrimidin-2-yl)piperazin-1-yl]-(4-²H₂)butyl}-(2,6-²H₂)-4-azatricyclo[5.2.1.0^{2,6}]decane-3,5-dione (XV) and (1R,2R,6S,7S)-4-{4-[4-(pyrimidin-2-yl)piperazin-1-yl]-(2,2,3,3,4,4-²H₆)butyl}-4-azatricyclo[5.2.1.0^{2,6}]decane-3,5-dione (XVIII).

The new analogues of tandospirone as defined by formula I are deuterated in specific positions and can thus be metabolically stabilized in order to reduce or delay metabolism and change the pattern of metabolism. The compounds of the current invention can be beneficial by having pharmacological properties comparable to non-deuterated tandospirone and additionally improved pharmacokinetic properties compared to the non-deuterated tandospirone. The improvement of pharmacokinetic properties is obtained because deuterated analogues of tandospirone can have reduced formation of metabolites after administration and can therefore be associated with less risk of adverse effects compared to non-deuterated tandospirone. In particular the compounds of the current invention may be beneficial due to their pharmacokinetic properties, which include a fast onset of action, a long duration of action and increased exposure. Furthermore the compounds of the current invention can be beneficial due to their lower propensity to affect the metabolism of other drugs (drug-drug interaction).

A reduction in metabolic clearance rate would potentially increase patient compliance by a reduction in number of daily doses needed and reduce fluctuation in tandospirone plasma concentrations outside the therapeutically relevant area to mitigate the risk of adverse events and drug exposure.

In one embodiment of the present invention, pharmacokinetic properties of the deuterated compounds are improved in a manner that allows for an alteration in the administration profile of the deuterated compounds compared to non-deuterated tandospirone, such as for example from administration 3 times per day to 2 times per day, or 2 times per day to 1 time per day.

The stability and metabolism of compounds can be measured by assays involving human liver microsomes. When different compounds are compared, the half-life (T½) and the rate of intrinsic clearance can be computed and used for comparison. An altered, inhibited or delayed metabolism can be observed when the half-life of a deuterated tandospirone compound is increased compared to non-deuterated tandospirone, or when the rate of intrinsic clearance is decreased for deuterated tandospirone compound compared to non-deuterated tandospirone.

In a preferred embodiment of the present invention, the rate of intrinsic clearance (Clint) is decreased for the deuterated tandospirone compound compared to tandospirone, and even more preferably significantly decreased compared to tandospirone. Thus in such an embodiment, the deuterated tandospirone compounds have an intrinsic clearance rate in the range of 1 ml/min/kg to 540 ml/min/kg, such as in the range of 1-260 ml/min/kg, even more preferably such as in the range of 50-250 ml/min/kg.

In another preferred embodiment, when the rate of intrinsic clearance (Clint) is decreased for the deuterated tandospirone compound compared to tandospirone, the deuterated tandospirone compounds have an intrinsic clearance rate which is 0-90% of the intrinsic clearance rate of tandospirone, preferably in the range of 50-90%.

### Plasma Protein Binding:

The pharmacokinetic profile of a drug may be affected by the degree to which it binds to the proteins within blood plasma. The less bound a drug is, the more efficiently it can potentially traverse cell membranes or diffuse into the site of action. In one embodiment of the present invention, the deuterated compounds have a reduced plasma protein binding compared to tandospirone and the plasma protein binding is range of 1-99% compared to the binding measured for tandospirone, preferably 1-83% compared to the binding measured for tandospirone. Thus when the plasma protein binding of tandospirone is measured to 70%, the plasma protein binding of the deuterated compounds is in the range of 1-69% binding, more preferably significantly reduced and in the range of 1-62% binding, even more preferably in the range of 40-62% plasma protein binding.

The ability of compounds to pass a biological membrane (permeability), like the gastro-intestinal epithelia and the blood-brain barrier endothelia is of importance for orally delivered drugs and for compounds targeting receptors in the brain. Methods for measuring biomembrane permeability are known in the art. For example assays using the Madin-Darby Canine Kidney (MDCK) cell line are used as an industry standard to evaluate biomembrane passage properties of compounds; the apparent biomembrane permeability can be measured using such an assay. In one embodiment of the present invention, the apparent biomembrane permeability of the deuterated tandospirone compound is increased compared to tandospirone, such as significantly increased and in the range of 33×10⁻⁶ cm/s to 100×10⁻⁶ cm/s.

In a particular embodiment of the present invention, the metabolism mediated by CYP3A4 or CYP2D6 is altered, inhibited or delayed for new deuterated tandospirone analogues compared to non-deuterated tandospirone.

In another embodiment of the present invention the metabolism mediated by CYP3A4, CYP2C9, CYP2C19, CYP1A2 or CYP2D6 is altered, inhibited or delayed for new deuterated tandospirone analogues compared to non-deuterated tandospirone.

Inhibition of the main drug metabolizing enzymes in human liver may lead to clinically significant drug-drug interactions. If two drugs are given in combination and are metabolised by the same enzymes, competition for metabolism may give rise to increased plasma concentrations and therefore possible adverse effects (Lin et al., 1997). The inhibitory potential of tandospirone and the deuterated tandospirone compounds of this invention can for example be tested in assays using the cytochrome P450 enzyme, CYP3A4 that is most frequently associated with drug metabolism and constitute the quantitative majority of P450 enzymes in the human liver (Shimada et al., 1994). Such assays are described herein. CYP3A4 is the major enzyme involved in tandospirone metabolism (Niwa et al., 2005). Thus in one embodiment of the present invention, the deuterated tandospirone compounds have decreased inhibition of the metabolism mediated by CYP3A4 of test-compounds such as midazolam compared to the inhibition of tandospirone. In such an embodiment, the IC₅₀ of deuterated tandospirone compounds are in the range of 28-200 μM, and more preferably in the range of 35-80 μM. Alternatively, in such an embodiment, the IC₅₀ of a deuterated tandospirone compound is increased by 10-100%, preferably 20-80% compared to the IC₅₀ of tandospirone.

The metabolism of the compounds can for instance be measured by incubations with recombinant human CYP enzymes as described in the present examples. In one example of CYP enzyme assays, the degradation/metabolism of non-deuterated tandospirone or deuterated tandospirone analogues can be measured as the decrease/difference in the amount of compound remaining after incubation with enzymes relative to the amount of compound remaining after incubation with enzyme and enzyme inhibitor or relative to the amount of compound before incubation. By comparing the size of said decrease/difference in the amount of non-deuterated tandospirone with the size of the decrease/difference for the deuterated tandospirone analogues the metabolism of the compounds can then be observed, and the difference in decrease of compound can be measured.

An altered, inhibited or delayed metabolism can be observed when the decrease in the amount of compound observed after incubation with enzymes is smaller for the deuterated tandospirone than for non-deuterated tandospirone. Thus in one embodiment, the measured decrease in amount of compound after CYP enzyme incubation is smaller for deuterated tandospirone analogues compared to non-deuterated tandospirone, for example in the range of 0.05% to 50% smaller, such as 0.05% to 10% smaller, such as 0.05% to 5% smaller, or such as 5% to 10% smaller, or such as 10% to 20% smaller, such as 10% to 15% smaller or such as 15% to 20% smaller, or such as 20% to 30% smaller, such as 20% to 25% smaller or such as 25% to 30% smaller, or such as 30% to 40% smaller, such as 30% to 35% smaller or such as 35% to 40% smaller, or such as 40% to 50% smaller, such as 40% to 45% smaller or such as 45% to 50% smaller, or for example in the range of 50% to 80% smaller, such as 50% to 60% smaller, or such as 60% to 70% smaller, or such as 70% to 80% smaller, or such as 80 to 100% smaller.

In one embodiment of the present invention, the amount of the deuterated compound measured in percent after incubation with CYP enzymes is higher than the amount of non-deuterated compound measured in percent after incubation with CYP enzymes, such as 0.05% to 100% higher, for example such as 0.05% to 0.5% higher, or for example such as 0.5% to 50% higher, such as 0.5% to 1% higher, or such as 1% to 5% higher, or such as 5% to 10% higher, or such as 10% to 15% higher, or such as 15% to 20% higher, or such as 20% to 25% higher, or such as 25% to 30% higher, or such as 30% to 35% higher, or such as 35% to 40% higher, or such as 40% to 45% higher, or such as 45% to 50% higher or for example such as 50% to 100% higher, such as 50% to 55% higher, or such as 55% to 60% higher, or such as 60% to 65% higher, or such as 65% to 70% higher, or such as 70% to 75% higher, or such as 75% to 80% higher, or such as 80% to 85% higher, or such as 85% to 90% higher, or such as 90% to 95% higher, or such as 95% to 100% higher.

In another embodiment, the amount of the deuterated compound measured in percent after incubation with CYP enzymes is 0.5% to 50% higher than the amount of non-deuterated compound measured in percent after incubation with CYP enzymes.

In one embodiment of the present invention, the amount of deuterated-compound is measured in percent after incubation with the CYP3A4 enzyme is increased by 0.5% to 50% compared to non-deuterated tandospirone.

Metabolites of deuterated tandospirone analogues may be deuterium labeled metabolites or non-deuterated metabolites, depending on the specific reactions of metabolism. Metabolic reactions may involve positions in the compounds according to the present invention which are not designated as deuterium or may involve positions which are designated as deuterium.

### Medical Indications

The compounds of the present invention possess the similar pharmacological activity as tandospirone free base and its salts, such as tandospirone citrate, and are useful for treating that can be ameliorated with agonists of the serotonin 5-HT1A receptors. The present invention provides compounds, pharmaceutical compositions and methods for treatment of diseases wherein activation of the serotonin 5-HT1A receptor will have a beneficial therapeutic effect, or diseases which are associated with dysfunction of the serotonin 5-HT1A receptor.

In a preferred embodiment the compounds of the present invention are used for treatment of various types of skin disorders or conditions, such as atopic dermatitis, seborrhoeic dermatitis, diaper dermatitis, allergic contact dermatitis, irritant contact dermatitis, unspecified contact dermatitis, infective dermatitis, exfoliative dermatitis, lichen simplex chronicus, lichen planus, pruritus/itch, pityriasis rosea, rosacea, psoriasis, urticaria (allergic and unspecified), erythema, sunburn, pemphigus and other acantholytic disorders.

Atopical dermatitis is an inflammatory, chronically relapsing, non-contagious and pruritic skin disorder, which is also named “infantile eczema” because the disorder is normally developed in young children. Approximately 50% of the patients who develop the condition display symptoms before the age of 1, and 80% display symptoms within the first 5 years of life. In some instances, the disorder may persist into adulthood or symptoms may develop later in life.

In a particular embodiment of the present invention the compounds, pharmaceutical compositions and methods for treatment are for treatment of atopical dermatitis.

In another preferred embodiment the compounds are used for treatment of dermatological disorders associated with stress and treatment of dermatological disorders associated with diseases of the central nervous system such as anxiety and depressions.

In another embodiment the compounds of the present invention is used for treatment of disorders of the central nervous system, such as anxiety, panic disorder, obsessive-compulsive disorder (OCD) and post-traumatic stress disorder (PTSD), depressions, schizophrenia, akathesia induced by neuroleptica, ADHD, Machado-Joseph disease, Parkinsons disease and symptoms associated with treatment of Parkinsons disease; in particular dyskinesia associated with treatment of Parkinson's disease with L-DOPA, movement disorders; in particular blepharospasm and chorea and disorders associated with choreic movements, addiction and abuse especially related to abuse of cocaine, methamphetamine, alcohol and/or tobacco smoking, impairments and dysfunctions caused by cerebral ischemia.

In yet another embodiment, the compounds of the present invention are used for treatment of disorders of the central nervous system such as movement disorders, such as disorders which are associated with altered or impaired synaptic dopamine levels. Movement disorders according to the present invention may be selected from the group of disorders comprising ataxia, akathisia, dystonia, essential tremor, Huntington's disease, myoclonus, Parkinson's disease, Rett syndrome, tardive dyskinesia, Tourette syndrome, Wilson's disease, dyskinesia, chorea, Machado-Joseph disease, restless leg syndrome, spasmodic torticollis, geniospasm, or movement disorders associated therewith.

In a preferred embodiment the compounds of the present invention is used for treatment of cognitive impairment/dysfunction disorders such as cognitive impairment associated with schizophrenia (CIAS); schizophrenia; dementias; autism; ADHD; and Alzheimer's disease. The compounds of the invention are also expected to treat positive and negative aspects of schizophrenia, dementia, autism, ADHD, and Alzheimer's disease.

Since serotonin 5-HT1A agonists have been shown to affect food intake (there are reports that indicate that such compounds either increase or reduce food intake) compounds of the present invention can be used to treat eating disorders such as bulimia, bulimia nervosa, binge eating disorders, and night eating disorders.

In one embodiment the compounds of the present invention is used for treatment of development of tolerance to the treatment effects of morphine and opiates, treatment of alcohol and smoking dependence, treatment of dyspepsia, acute, chronic and idiopathic cough, age related macular degeneration (AMD) and sexual dysfunction.

In one embodiment of the present invention, the compounds or pharmaceutical compositions as defined herein are used for treatment of pain, such as acute pain, chronic pain, visceral pain and neuropathic pain.

In another embodiment of the present invention, the compounds or pharmaceutical compositions as defined herein are used for treatment of postoperative nausea and/or vomiting (PONV).

In yet another embodiment of the present invention, the compounds or pharmaceutical compositions as defined herein are used for treatment of alcohol and smoking dependence.

In some embodiments of the present invention, the compounds or pharmaceutical compositions of the present invention are part of a combination therapy, and may be administered in combination with one or more second active ingredients or agents which may have beneficial therapeutic effect on the medical conditions described herein. Said agents may be administered at the same time (simultaneously), where they may be combined in a single dosage form (a pharmaceutical composition for simultaneous administration), or at a different time (sequentially) as separate compounds.

In one embodiment of the present invention, the use of compounds or pharmaceutical compositions of the present invention in combination with one or more second active ingredients can have a useful dose-sparing effect, and thus lower the required dosage of the second active ingredient used in combination with the compounds of the present invention.

In one preferred embodiment of the present invention, the second active ingredient is a modulator of serotonin receptors or a serotonin reuptake inhibitor such as e.g. fluoxetine, fluvoxamine, paroxetine, sertraline, citalopram, escitalopram and venlafaxine.

In some preferred embodiments pharmaceutical compositions of the compounds of the present invention are used for treatment of atopical dermatitis or other dermatological disorders. Thus according to second preferred embodiment of the present invention, the compounds of the invention are administered in combinations with corticosteroid wherein said corticosteroid may be administered as oral formulations or as creams or ointments; with antibiotics; with antihistamines; with certain immunomodulators such as tacrolimus and pimecrolimus; with vitamin derivatives such as the vitamin A and vitamin D₃ analogues; with certain biologics such as biologics able to bind tumor necrosis factor α (TNF-α) for example monoclonal antibodies of TNF-α, adalimumab and etanercept; with NK-1 antagonists; with light therapy.

Compounds or pharmaceutical compositions as defined herein can be used for treatment of different types of pain. Thus in one embodiment of the present invention, compounds or pharmaceutical compositions of the present invention are used in combination with a second active ingredient selected from the group of other analgesic medication classes, such as strong and weak opioids, NSAIDs, COX-2 inhibitors, acetaminophen, other anti-inflammatory, tricyclic antidepressants, anticonvulsant agents, voltage gated calcium channel blockers, N-type calcium channel blockers, other calcium channel modulators, SNRI and other monoamine reuptake inhibitors, sodium channel blockers, NMDA antagonists, AMPA antagonists, other glutamate modulators, GABA modulators, CRMP-2 modulators, NK-1 antagonists, TRPV1 agonists, cannabinoids, adenosine agonists, nicotinic agonists, p38 MAP kinase inhibitors, corticosteroids, and other analgesic drug classes such as triptans used for treatment and prevention of migraine.

Strong and weak opioids according to the present invention can be selected from the list consisting of fentanyl, oxycodone, codeine, dihydrocodeine, hydrocodone, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papvereturn, alfentanil, buprenorphine and tramadol, or other opioids known in the art, and pharmaceutically acceptable derivates, homologs or analogs thereof and combinations thereof.

Among the preferred strong or weak opioids which are useful for administration in combination with compounds of the present invention are hydrocodone, oxycodone, codeine or tramadol and other strong opiods.

In one embodiment of the present invention, the compounds or pharmaceutical compositions as defined herein are used in combination with at least one other antiemetic agent comprising one or more compounds selected from 5HT3 antagonists, such as for example granisetron, ondansetron, tropisetron, palonosetron, ramosetron or dolasetron, NK-1 antagonists such as for example aprepitant or casopitant, dopamine antagonists such as for example domperidone, droperidol, haloperidol, clopromazine, or prochlorperazine, H1 histamine receptor antagonists such as for example cyclizine, diphenhydramine, dimenhydrinate, meclizine, promethazine, or hydroxyzine, cannabinoids such as for example tetrahydrocannabinol, dronabinol, or nabilone, benzodiazepines such as for example midazolam or lorazepam, anticholinergic compounds such as for example scopolamine or steroid compounds such as for example dexamethasone.

In another embodiment, the compounds or pharmaceutical compositions of the present invention are useful for administration in combination with other medical agents used in treatment of alcohol or smoking dependence, such as nalmefene, naltrexone, acamprosate, disulfuram, levetiracetam, divalproex, quetiapine combined with haloperidol, quetiapine, quetiapine combined with topiramate, flumazenil combined with gabapentin, olanzapine combined with ondansetron, eszopiclone, rimonabant, topiramate, sertraline, MK-0594, SCH-900435, Org 25935, LY2196044, ALKS 29, ALKS 33, 598809 (GSK), LY2456302, LY2371712, DOV 102677, 618334 (GSK) and the medication class of NK-1 receptor antagonists.

In yet another embodiment. the compounds or pharmaceutical compositions of the present invention are useful for administration alone and/or in combination with other medical agents used in treatment of for treatment of eating disorders, such as for example binge eating, and can be administered in combination with memantine, lamotrigine, sodium oxybate, acamprosate, buproprion, duloxetine, sibutramine, rimonabant, and the medication class of NK-1 receptor antagonists.

In yet another embodiment. the compounds or pharmaceutical compositions of the present invention are useful for administration alone and/or in combination with other medical agents used in treatment of for treatment of cough including acute cough, chronic cough, and idiopathic cough, such as medicaments selected from the group of opioids and related medical agents including strong and weak opioids according to the present invention such as fentanyl, oxycodone, codeine, dihydrocodeine, hydrocodone, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papvereturn, alfentanil, buprenorphine and tramadol, or other opioids known in the art, and pharmaceutically acceptable derivates, homologs or analogs thereof and combinations thereof, NK-1 receptor antagonists, CB-2 receptor antagonists, TRPV1 agonists, medical agents as AG1321001, BIBW 2948 BS, PDC-748, SCH 486757, 443C81 and capsaicin.

### Degree of Deuterium Labelling

It will be recognized that some variations of the natural isotopic abundance occurs in a synthesized compound depending upon the origin of chemical materials used in the synthesis. Thus, a preparation of any compound will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See for instance: Gannes L Z et al. 1998.

In a compound of this invention, when a particular position is designated as including deuterium, it is understood that the abundance of deuterium at that position is substantially greater than the natural abundance of deuterium, which is 0.015%. Thus, in compounds of larger deuterium abundance, the isotopic enrichment factor is significantly above 1. A position designated as having deuterium according to the present invention typically has a minimum isotopic enrichment factor of at least 3000 (45% deuterium incorporated) at each atom designated as deuterium in said compound. Unless otherwise stated, when a position is designated specifically as “H” or “hydrogen”, the position is understood to have hydrogen at its natural abundance isotopic composition. In the compound of this invention any atom not specifically designated as particular isotope is meant to represent any stable isotope of that atom. In one embodiment of the present invention, the pharmaceutical composition of the invention comprises the compounds disclosed herein which have an isotopic enrichment factor for each designated deuterium atom of at least 3333 (50% deuterium incorporated at each position designated as a deuterium atom), such as at least 3500 (52.5% deuterium incorporated at each position designated as a deuterium atom), such as at least 3666 (55% deuterium incorporated at each position designated as a deuterium atom), such as at least 4000 (60% deuterium incorporation at each position designated as a deuterium atom), such as at least 4333 (65% deuterium incorporation at each position designated as a deuterium atom), such as at least 4500 (67.5% deuterium incorporation at each position designated as a deuterium atom), such as at least 4666.6 (70% deuterium incorporation at each position designated as a deuterium atom), such as at least 5000 (75% deuterium incorporation at each position designated as a deuterium atom), such as at least 5333 (80% deuterium incorporation at each position designated as a deuterium atom), such as at least 5500 (82.5% deuterium incorporation at each position designated as a deuterium atom), such as at least 5666 (85% deuterium incorporation at each position designated as a deuterium atom), such as at least 6000 (90% deuterium incorporation at each position designated as a deuterium atom), such as at least 6333.3 (95% deuterium incorporation at each position designated as a deuterium atom), such as at least 6400 (96% deuterium incorporation at each position designated as a deuterium atom), such as at least 6466.7 (97% deuterium incorporation at each position designated as a deuterium atom), such as at least 6533.3 (98% deuterium incorporation at each position designated as a deuterium atom), such as at least 6600 (99% deuterium incorporation at each position designated as a deuterium atom), such as at least 6633 (99.5% deuterium incorporation at each position designated as a deuterium atom), such as at least 6660 (99.9% deuterium incorporation at each position designated as a deuterium atom).

In a preferred embodiment of the present invention, the isotopic enrichment factor is at least 6000 (90% deuterium incorporation at each position designated as a deuterium atom), such as at least 6333.3 (95% deuterium incorporation at each position designated as a deuterium atom), such as at least 6600 (99% deuterium incorporation at each position designated as a deuterium atom).

In another embodiment of the present invention, the pharmaceutical composition of the invention comprises the compounds disclosed herein wherein the abundance of hydrogen in the positions designated as deuterium is less than 49.9%, such as less than 45%, such as less than 40%, such as less than 35%, such as less than 30%, such as less than 25%, such as less than 20%, such as less than 15%, such as less than 12.5%, such as less than 10%, such as less than 9%, such as less than 8%, such as less than 7%, such as less than 6%, such as less than 5%, such as less than 4%, such as less than 3%, such as less than 2%, such as less than 1%, such as less than 0.5%, such as less than 0.01%.

The pharmaceutical composition according to the present invention may also comprise compounds according to formula I wherein deuterium is incorporated to a varying degree in any of the positions R1, R2, R3, R10 and R11. In one embodiment of the present invention at least 50% of the compounds are isotopically enriched with deuterium, such as in at least 55% of the compounds isotopically enriched with deuterium, such as at least 60% of the compounds isotopically enriched with deuterium, such as at least 65% of the compounds isotopically enriched with deuterium, such as at least 70% of the compounds isotopically enriched with deuterium, such as at least 75% of the compounds isotopically enriched with deuterium, such as at least 80% of the compounds isotopically enriched with deuterium, such as at least 85% of the compounds isotopically enriched with deuterium, such as at least 90% of the compounds isotopically enriched with deuterium, such as at least 95% of the compounds isotopically enriched with deuterium, such as at least 96% of the compounds isotopically enriched with deuterium, such as at least 97% of the compounds isotopically enriched with deuterium, such as at least 98% of the compounds isotopically enriched with deuterium, such as at least 99% of the compounds isotopically enriched with deuterium, such as at least 99.5% of the compounds isotopically enriched with deuterium, such as at least 99.9% of the compounds isotopically enriched with deuterium.

The pharmaceutical composition according to the present invention may also comprise compounds according to formula I wherein deuterium is incorporated to a varying degree in any of the positions R1, R2, R3, R4, R5, R6, R7, R8, R9, R10 and R11. Thus in one embodiment of the present invention, pharmaceutical compositions are compositions comprising deuterated compounds as defined herein wherein at least 50% of the compounds are isotopically enriched with deuterium, such as in at least 55% of the compounds isotopically enriched with deuterium, such as at least 60% of the compounds isotopically enriched with deuterium, such as at least 65% of the compounds isotopically enriched with deuterium, such as at least 70% of the compounds isotopically enriched with deuterium, such as at least 75% of the compounds isotopically enriched with deuterium, such as at least 80% of the compounds isotopically enriched with deuterium, such as at least 85% of the compounds isotopically enriched with deuterium, such as at least 90% of the compounds isotopically enriched with deuterium, such as at least 95% of the compounds isotopically enriched with deuterium, such as at least 96% of the compounds isotopically enriched with deuterium, such as at least 97% of the compounds isotopically enriched with deuterium, such as at least 98% of the compounds isotopically enriched with deuterium, such as at least 99% of the compounds isotopically enriched with deuterium, such as at least 99.5% of the compounds isotopically enriched with deuterium, such as at least 99.9% of the compounds isotopically enriched with deuterium.

Thus, the current invention relates to pharmaceutical compositions comprising novel tandospirine analogues for which one or more protons have been substituted with deuterium.

It can be quite difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enrichment can be achieved by either exchanging protons with deuterium or by synthesizing the molecule with enriched starting materials.

The relative amounts of impure isotopologues in a pharmaceutical composition of this invention will depend upon a number of factors including the isotopic purity of deuterated reagents used to make the compound or pharmaceutical composition and the efficiency of incorporation of deuterium in the various synthesis steps used to prepare the compound or pharmaceutical composition. However, as set forth above the relative amount of such impure isotopologues will be less than 49.9% of the pharmaceutical composition.

The relative amounts of isotopologues in a compound of this invention depend upon a number of factors including the isotopic purity of deuterated reagents used to make the compound and the efficiency of incorporation of deuterium in the various synthesis steps used to prepare the compound. However, as set forth above the relative amount of such isotopologues will be less than 49.9% of the compound.

### Pharmaceutical Compositions

The compounds of the present invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington 2000.

A compound for use according to the present invention is generally utilized as the free substance or as a pharmaceutically acceptable salt thereof. Examples of the latter are: an acid addition salt of a compound having a free base functionality, and a base addition salt of a compound having a free acid functionality, as well as pharmaceutically acceptable metal salts, ammonium salts and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acid, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic and p-toluenesulfonic acid, and the like. Further examples of pharmaceutically acceptable inorganic or organic acid addition salts include the pharmaceutically acceptable salts listed in J. Pharm. Sci. 1977, 66, 2, the contents of which are incorporated herein by reference. Examples of metal salts include lithium, sodium, potassium and magnesium salts, and the like. Examples of ammonium and alkylated ammonium salts include ammonium, methylammonium, dimethylammonium, trimethylammonium, ethylammonium, hydroxyethylammonium, diethylammonium, butylammonium and tetramethylammonium salts, and the like.

In one aspect of the present invention, deuterated tandospirone analogues are on crystalline forms, for example co-crystallized forms or hydrates of crystalline forms.

A therapeutically effective amount of a compound according to the present invention is an amount sufficient to cure, alleviate or partially arrest the clinical manifestations of a given disease or disorder and its complications. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or veterinary.

Pharmaceutical compositions according to the present invention may be specifically formulated for administration by any suitable route, such as the oral, rectal, nasal, pulmonary, buccal, sublingual, transdermal, intracisternal, intraperitoneal, and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use. Depot injectable formulations are also regarded as being within the scope of the present invention.

Other suitable administration forms include suppositories, sprays, ointments, cremes, gels, inhalants, dermal patches, implants, etc.

A typical oral dosage is in the range of from about 0.001 to about 100 mg/kg body weight per day, preferably from about 0.01 to about 50 mg/kg body weight per day, and more preferably from about 0.05 to about 10 mg/kg body weight per day, administered in one or more doses such as 1-3 doses. The exact dosage will depend upon the frequency and mode of administration, the sex, age, weight and general condition of the subject treated the nature and severity of the condition treated and any concomitant diseases to be treated, and other factors evident to those skilled in the art. The formulations may conveniently be prepared in unit dosage form by methods known to those skilled in the art. A typical unit dosage form for oral administration one or more times per day, such as 1-3 times per day, may contain from 0.05 to about 1000 mg, preferably from about 0.1 to about 500 mg, and more preferably from about 0.5 mg to about 200 mg of a compound of the invention.

For parenteral routes such as intravenous, intrathecal, intramuscular and similar administration, typical dosages are in the order of about half the dosage employed for oral administration.

For parenteral administration, solutions of compounds for use according to the present invention in sterile aqueous solution, in aqueous propylene glycol or in sesame or peanut oil may be employed. Aqueous solutions should be suitably buffered where appropriate, and the liquid diluent rendered isotonic with, e.g., sufficient saline or glucose. Aqueous solutions are particularly suitable for intravenous, intramuscular, subcutaneous and intraperitoneal administration. The sterile aqueous media to be employed are all readily available by standard techniques known to those skilled in the art.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Moreover, the carrier or diluent may include any sustained release material known in the art, such as glyceryl monostearate or glyceryl distearate, alone or mixed with a wax. The pharmaceutical compositions formed by combining the compounds for use according to the present invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units, such as capsules or tablets, which each contain a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents and preserving agents in order to provide pharmaceutically elegant and palatable preparations. Tablets may contain the active ingredient(s) in admixture with non-toxic pharmaceutically acceptable excipients which are suitable for the manufacture of tablets. These excipients may, for example, be: inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, the contents of which are incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil. Aqueous suspensions may contain the compound for use according to the present invention in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising compounds for use according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agent. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol.

Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include, for example, cocoa butter and polyethylene glycols.

For buccal and sublingual use, creams, ointments, jellies, solutions of suspensions, etc., containing the compounds of the invention may be employed. In the context of the present invention, formulations for buccal and sublingual application include mouth washes and gargles.

Compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some compounds of the present invention may form solvates with water or common organic solvents. Such solvates are also encompassed within the scope of the invention.

Thus, a further embodiment provides a pharmaceutical composition comprising a compound for use according to the present invention, or a pharmaceutically acceptable salt, solvate, or prodrug thereof, and one or more pharmaceutically acceptable carriers, excipients, or diluents.

If a solid carrier is used for oral administration, the preparation may be tabletted, placed in a hard gelatine capsule in powder or pellet form, or may be in the form of a troche or lozenge. The amount of solid carrier will vary widely, but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet that may be prepared by conventional tabletting techniques may contain:

Core:

| | |
|---|---|
| Active compound (as free compound or salt thereof) | 5.0 mg |
| Lactosum Ph. Eur. | 67.8 mg |
| Cellulose, microcryst. (Avicel) | 31.4 mg |
| Amberlite ®^{□}IRP88* | 1.0 mg |
| Magnesii stearas Ph. Eur. | q.s. |

| | |
|---|---|
| Hydroxypropyl methylcellulose | approx. 9 mg |
| Mywacett 9-40 T** | approx. 0.9 mg |

*Polacrillin potassium NF, tablet disintegrant, Rohm and Haas.
**Acylated monoglyceride used as plasticizer for film coating.

If desired, the pharmaceutical composition comprising a compound according to the present invention may additionally comprise further active substances, such as those described in the foregoing.

It is an object of the present invention to provide formulations of the compounds according to formula I which allow for administration of a therapeutically effective amount of the compounds to an individual in need. Such amounts may vary according to the frequency and mode of administration, the sex, age, weight and general condition of the subject treated, the nature and severity of the condition treated and or other treatments used by the individual, and may be determined by conventional techniques in the field.

### Methods for Treatment

Methods for treatment according to the present invention comprise at least one step of administration of the compounds according to the present invention to an individual in need thereof. Said step includes separate, sequential or/and simultaneous administration of a therapeutically effective amount of pharmaceutical compositions according to the present invention to an individual in need thereof. Such methods may further include at least one step of administration of a second active ingredient.

The methods for treatment according to the present invention comprises steps wherein the compounds according to the present the invention are administered in a therapeutically effective amount, such as doses of 0.001 to 1000 mg, such as 0.01 to 600 mg, or such as 0.5 mg to 200 mg per day.

In one embodiment of the present invention, the deuterated compounds have increased stability and/or altered pharmacokinetic profile which allows for an administration which is different from tandospirone, such as for example administration 1 time per day or 2 times per day, or administration in doses which are smaller than doses used for tandospirone.

In one embodiment of the present invention, the individuals in need of treatment are of various age such as 0 to 120 years, for example infants or children in age of 0 to 5 years, for example children of the age 5 to 12 years, for example children in the age of 12 to 18 years, for example adults of the age 18 to 25 years, for example adults of the age 25 to 40 years, for example adults of the age 40 to 60 years, for example adults of the age 60 to 80 years, for example adults of the age 80 to 100 years, or for example adults of the age 100 to 120 years.

In a particular embodiment of the present invention, the individuals in need of treatment are children in the age of 0 to 18 years.

A scoring system such as for example the SCORing Atopic Dermatitis (SCORAD) Index may be used as part of methods for treatment according to the present invention. Such a scoring system may be used for selecting individuals in need of treatment by indexing individuals prior to treatment, and/or monitoring effects of treatment by scoring the individuals during the treatment period. For example the severity of the disease can assessed before the first treatment and at days 15 and 29 of the treatment period by the investigating physician using the SCORAD index.

Another scoring system or measure for atropic dermatitis may be itching rated by the patients themselves using a visual analogue scale (VAS) one or more times a day. An example of a visual analogue scale rating can be that the patients are asked to grade the current itching on a 10 cm visual analogue scale. The ends of the scale are labeled “no itching” (0 cm) and “worst itching” (10 cm) and the severity of the disease and/or the effects of treatment are monitored by the grading on the scale.

### Kit of Parts

It is an object of the present invention, to provide a kit of parts comprising the compounds according to formula I or pharmaceutical compositions according to the invention for simultaneous, sequential or separate administration. The kit of parts is useful in the methods for treatment according to the present invention. Such kit of parts may further include second active ingredients as defined herein, for simultaneous or sequential administration.

### Methods for Preparation of Compounds

The present invention also provides methods for the preparation of compounds according to formula I.

In one embodiment of the present invention, a method is provided for synthesis of a deuterated compound according to formula I

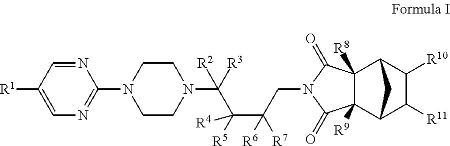

wherein R1, R2, R3, R10 and R11 are independently selected from the group consisting of hydrogen (H) and deuterium (D) with the proviso that at least one of R1, R2, R3, R10 and R11 is hydrogen and wherein said method comprises one or more of the following steps:
a) treating a mixture of reagent and 50% water-containing 10% palladium on charcoal in tetrahydrofuran (THF) by using D₂ gas,
b) stirring a solution of reagents and formalin-D₂ in dioxane and further adding drop wise a solution of copper sulphate in D₂O to form a mixture. Subsequently stirring mixture, followed by concentration in vacuo and treatment with toluene to obtain a product which is further filtered and concentrated in vacuo,
c) hydrogenation over 10% palladium on charcoal of a reagent to give a mixture, subsequently filtering said mixture and concentrating said filtrate in vacuo to give a product,
d) mixing reagents by stirring a mixture with anhydrous K₂CO₃ and KI in anhydrous DMF, followed by stirring, cooling to room temperature, pouring mixture into water and separating the product in the organic phase using EtOAc. Subsequently drying said organic phase and concentrating said product in vacuo,
e) refluxing a mixture of reagent, dibromobutane and anhydrous K₂CO₃ in acetone prior to cooling and filtration. Subsequently concentrating filtrate in vacuo,
f) refluxing a mixture of reagents and pyridine, followed by cooling, and concentration of the obtained product in vacuo,
g) refluxing a mixture of reagent, propagyl bromide and anhydrous K₂CO₃ in anhydrous acetone under nitrogen, followed by cooling and filtration, subsequent concentration of filtrate in vacuo and recrystallization from n-hexane to give a product.

According to the methods for preparation of compounds provided by present invention, said reagents may be selected from the group comprising 1-(5-bromopyrimidin-2-yl)piperazin, (1R,2S,6R,7S)-4-(4-bromobutyl)-4-azatricyclo[5.2.1.0^{2,6}]decane-3,5-dione, 1-((5-²H)pyrimidin-2-yl)piperazin, (1R,2R,6S,7S)-4-{4-[4-(5-bromopyrimidin-2-yl)piperazin-1-yl]butyl}-4-azatricyclo[5.2.1.0^{2,6}]decane-3,5-dione, (1R,2S,6R,7S)-4-(prop-2-yn-1-yl)-4-azatricyclo[5.2.1.0^{2,6}]decane-3,5-dione, (1R,2S,6R,7S)-4-{4-[4-(pyrimidin-2-yl)piperazin-1-yl]-(4-²H₂)but-2-yn-1-yl}-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]decane-3,5-dione, 4-oxatricyclo[5.2.1.0^{2,6}]dec-8-ene-3,5-dione, 4-[4-(pyrimidin-2-yl)piperazin-1-yl]-4-butan-1-amine, (8,9-²H₂)-4-oxatricyclo[5.2.1.0^{2,6}]decane-3,5-dione, 4-[4-(5-bromopyrimidin-2-yl)piperazin-1-yl]-4-butan-1-amine, (8,9-²H₂)-4-azatricyclo[5.2.1.0^{2,6}]decane-3,5-dione, (1R,2R,6S,7S)-4-{4-[4-(5-bromopyrimidin-2-yl)piperazin-1-yl]butyl}-(8,9-²H₂)-4-azatricyclo[5.2.1.0^{2,6}]decane-3,5-dione, (1R,2S,6R,7S)-4-azatricyclo[5.2.1.0^{2,6}]dec-8-ene-3,5-dione, 1-(5-bromopyrimidin-2-yl)piperazin, (1R,2S,6R,7S)-4-(4-bromobutyl)-4-azatricyclo[5.2.1.0^{2,6}]dec-8-ene-3,5-dione, (1R,2R,6S,7S)-4-{4-[4-(5-bromopyrimidin-2-yl)piperazin-1-yl]-(4-²H₂)butyl}-(8,9-²H₂)-4-azatricyclo[5.2.1.0^{2,6}]decane-3,5-dione, (1R,2S,6R,7S)-4-(prop-2-yn-1-yl)-4-azatricyclo[5.2.1.0^{2,6}]dec-8-ene-3,5-dione, of (1R,2S,6R,7S)-4-{4-[4-(pyrimidin-2-yl)piperazin-1-yl]-(4-²H₂)but-2-yn-1-yl}-4-azatricyclo[5.2.1.0^{2,6}]dec-8-ene-3,5-dione, (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione, (1R,2S,6R,7S)-4-azatricyclo[5.2.1.0^{2,6}]dec-8-ene-3,5-dione, (8,9-²H₂)-4-azatricyclo[5.2.1.0^{2,6}]decane-3,5-dione, (1R,2S,6R,7S)-4-(prop-2-yn-1-yl)-4(8,9-²H₂)-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione, (1R,2S,6R,7S)-4-(prop-2-yn-1-yl)-4-azatricyclo[5.2.1.0^{2,6}]dec-8-ene-3,5-dione, 1-((5-bromopyrimidin-2-yl)piperazin, (1R,2S,6R,7S)-4-{4-[4-(5-bromopyrimidin-2-yl)piperazin-1-yl]-(4-²H₂)but-2-yn-1-yl}-4-azatricyclo[5.2.1.0^{2,6}]dec-8-ene-3,5-dione, (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione.

According to the methods for preparation of compounds provided by present invention said products may be selected from the group comprising 4-((5-²H)pyrimidin-2-yl)piperazin, (1R,2R,6S,7S)-4-{4-[4-((5-²H)pyrimidin-2-yl)piperazin-1-yl]butyl}-4-azatricyclo[5.2.1.0^{2,6}]decane-3,5-dione, (1R,2S,6R,7S)-4-{4-[4-(pyrimidin-2-yl)piperazin-1-yl]-(4-²H₂)but-2-yn-1-yl}-4-azatricyclo[5.2.1.0^{2,6}]decane-3,5-dione, (1R,2R,6S,7S)-4-{4-[4-(pyrimidin-2-yl)piperazin-1-yl]-(4-²H₂)butyl}-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]decane-3,5-dione, ((1R,2R,6S,7S)-4-{4-[4-((5-²H)pyrimidin-2-yl)piperazin-1-yl]-(4-²H₂)butyl}-4-azatricyclo[5.2.1.0^{2,6}]decane-3,5-dione, (8,9-²H₂)-4-oxatricyclo[5.2.1.0^{2,6}]decane-3,5-dione, (1R,2R,6S,7S)-4-{4-[4-(pyrimidin-2-yl)piperazin-1-yl]butyl}-(8,9-²H₂)-4-azatricyclo[5.2.1.0^{2,6}]decane-3,5-dione, compound (1R,2R,6S,7S)-4-{4-[4-(5-bromopyrimidin-2-yl)piperazin-1-yl]butyl}-(8,9-²H₂)-4-azatricyclo[5.2.1.0^{2,6}]decane-3,5-dione, (1R,2R,6S,7S)-4-{4-[4-((5-²H)pyrimidin-2-yl)piperazin-1-yl]butyl}-(8,9-²H₂)-4-azatricyclo[5.2.1.0^{2,6}]decane-3,5-dione), (1R,2S,6R,7S)-4-(4-bromobutyl)-4-azatricyclo[5.2.1.0^{2,6}]dec-8-ene-3,5-dione, (1R,2R,6S,7S)-4-{4-[4-(5-bromopyrimidin-2-yl)piperazin-1-yl]-(4-²H₂)butyl}-(8,9-²H₂)-4-azatricyclo[5.2.1.0^{2,6}]decane-3,5-dione, (1R,2R,6S,7S)-4-{4-[4-((5-²H)pyrimidin-2-yl)piperazin-1-yl]butyl}-(8,9-²H₂)-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]dec-8-ene-3,5-dione, (1R,2S,6R,7S)-4-{4-[4-(pyrimidin-2-yl)piperazin-1-yl]-(4-²H₂)but-2-yn-1-yl}-(8,9-²H₂)-4-azatricyclo[5.2.1.0^{2,6}]decane-3,5-dione, (1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione, (8,9-²H₂)-4-azatricyclo[5.2.1.0^{2,6}]decane-3,5-dione, (1R,2S,6R,7S)-4-(prop-2-yn-1-yl)-4(8,9-²H₂)-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]dec-8-ene-3,5-dione, ((1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione, 1R,2S,6R,7S)-4-{4-[4-(5-bromopyrimidin-2-yl)piperazin-1-yl]-(4-²H₂)but-2-yn-1-yl}-4-azatricyclo[5.2.1.0^{2,6}]dec-8-ene-3,5-dione, (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione and ((1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione.

Such methods include methods as disclosed in the examples 1 to 10 of the present text.

### EXAMPLES

### General Procedures

The synthesis of tandospirone has been descibed in e.g. Ishizumi 1991.

Here below are examples of the synthesis of the new deuterated tandospirone analogues.

### Example 1

### (1R,2R,6S,7S)-4-{4-[4-((5-²H)pyrimidin-2-yl)piperazin-1-yl]butyl}-4-azatricyclo[5.2.1.0^{2,6}]decane-3,5-dione

### Method 1:

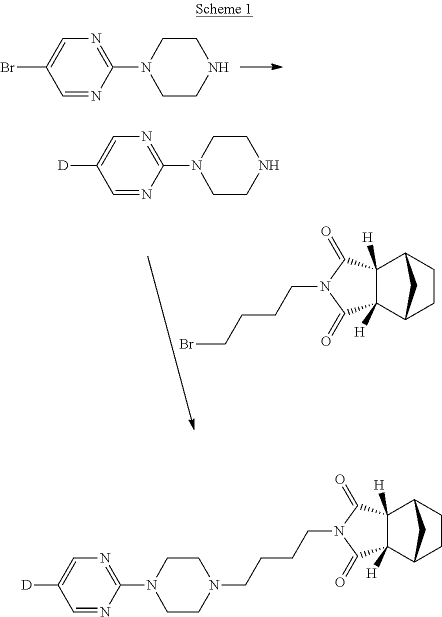

A mixture of 1-(5-bromopyrimidin-2-yl)piperazin (0.1 mol) and 50% water-containing 10% palladium on charcoal (0.8 g) in tetrahydrofuran (THF) is treated with D₂ gas at room temperature. Upon filtration the mixture is concentrated in vacuo to give 4-((5-²H)pyrimidin-2-yl)piperazin.

(1R,2S,6R,7S)-4-(4-bromobutyl)-4-azatricyclo[5.2.1.0^{2,6}]decane-3,5-dione (0.01 mol) is added to a stirred mixture of 1-((5-²H)pyrimidin-2-yl)piperazin (0.015 mol), anhydrous K₂CO₃ (0.015 mol), KI (0.0015 mol) in anhydrous DMF. The mixture is subsequently stirred at 90° C. for 1 hour, cooled to room temperature and poured into water. This mixture is separated between water and ethylacetate (EtOAc) whereupon the organic phase is washed with water and brine, dried and concentrated in vacuo. Purification by column chromatography and recrystallization gives the title compound ((1R,2R,6S,7S)-4-{4-[4-((5-²H)pyrimidin-2-yl)piperazin-1-yl]butyl}-4-azatricyclo[5.2.1.0^{2,6}]decane-3,5-dione).

### Method 2:

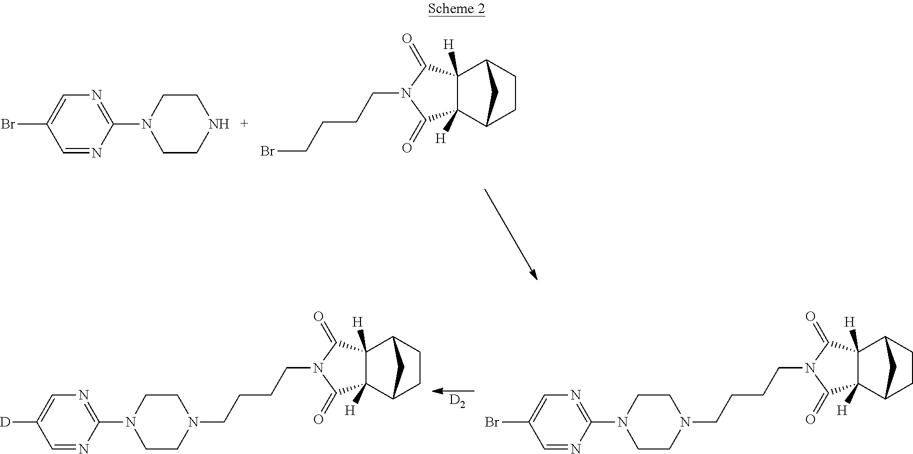

A mixture of (1R,2R,6S,7S)-4-{4-[4-(5-bromopyrimidin-2-yl)piperazin-1-yl]butyl}-4-azatricyclo[5.2.1.0^{2,6}]decane-3,5-dione (0.1 mol) and 50% water-containing 10% palladium on charcoal (0.8 g) in tetrahydrofuran (THF) is treated with D₂ gas at room temperature. Upon filtration the mixture is concentrated in vacuo to give the title compound ((1R,2R,6S,7S)-4-{4-[4-((5-²H)pyrimidin-2-yl)piperazin-1-yl]butyl}-4-azatricyclo[5.2.1.0^{2,6}]decane-3,5-dione).

### Example 2

### (1R,2R,6S,7S)-4-{4-[4-(pyrimidin-2-yl)piperazin-1-yl]-(4-²H₂)butyl}-4-azatricyclo[5.2.1.0^{2,6}]decane-3,5-dione

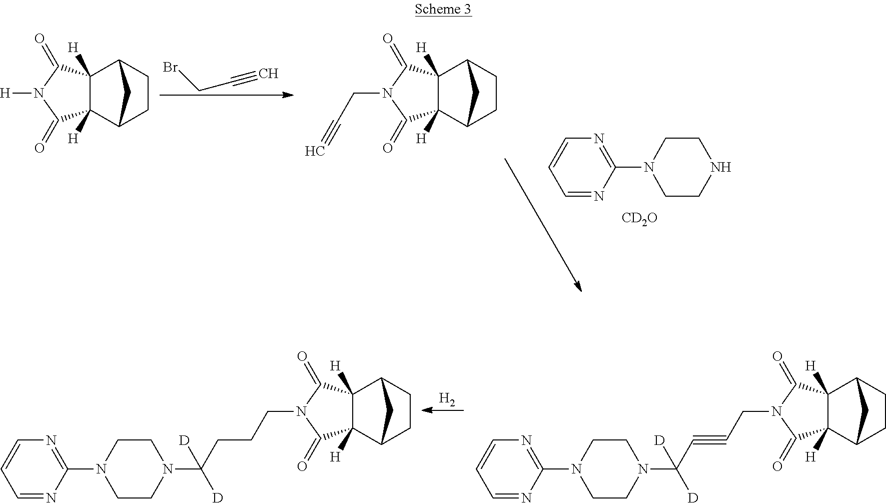

To a stirred solution of (1R,2S,6R,7S)-4-(prop-2-yn-1-yl)-4-azatricyclo[5.2.1.0^{2,6}]decane-3,5-dione (Ishizumi 1991) (1 mmol), 1-(2-pyrimidyl)piperazine (1 mmol) and 0.5 ml formalin-D₂ (20% w/w in D₂O (CDN Isotopes, UK)) in 2 ml dioxane is added drop wise a solution of copper sulphate (0.05 mmol) in D₂O (1 ml) at room temperature. The mixture is then stirred at 70-80° C. for 70 minutes, cooled to room temperature and concentrated in vacuo. The product is treated with toluene, filtered and concentrated in vacuo. Purification by column chromatography gives (1R,2S,6R,7S)-4-{4-[4-(pyrimidin-2-yl)piperazin-1-yl]-(4-²H₂)but-2-yn-1-yl}-4-azatricyclo[5.2.1.0^{2,6}]decane-3,5-dione.

A solution of (1R,2S,6R,7S)-4-{4-[4-(pyrimidin-2-yl)piperazin-1-yl]-(4-²H₂)but-2-yn-1-yl}-4-azatricyclo[5.2.1.0^{2,6}]decane-3,5-dione (0.5 mmol) in methanol (50 ml) is hydrogenated over 10% palladium on charcoal (10 mg) at 8 atm of hydrogen and 100° C. for 2 hours. The mixture is filtered and concentrated in vacuo. Purification by column chromatography gives the title compound ((1R,2R,6S,7S)-4-{4-[4-(pyrimidin-2-yl)piperazin-1-yl]-(4-²H₂)butyl}-4-azatricyclo[5.2.1.0^{2,6}]decane-3,5-dione).

### Example 3

### (1R,2R,6S,7S)-4-{4-[4-((5-²H)pyrimidin-2-yl)piperazin-1-yl]-(4-²H₂)butyl}-4-azatricyclo[5.2.1.0^{2,6}]decane-3,5-dione

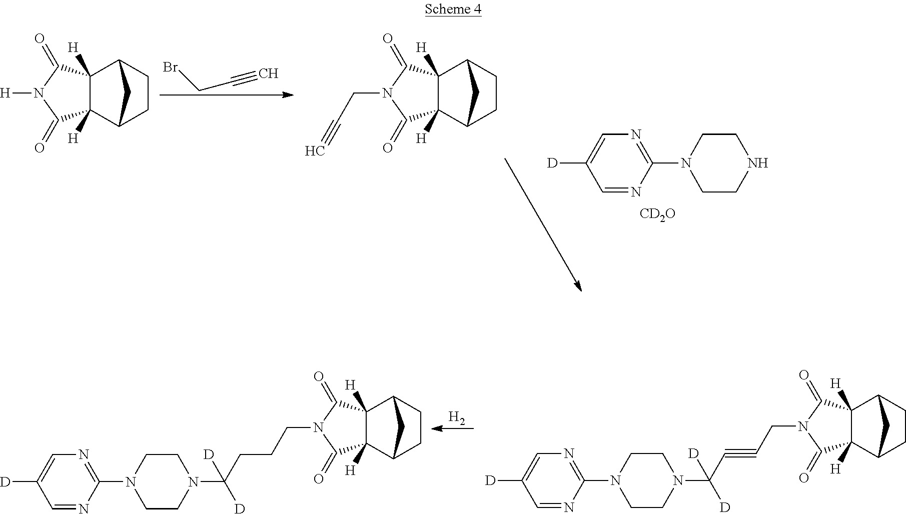

To a stirred solution of (1R,2S,6R,7S)-4-(prop-2-yn-1-yl)-4-azatricyclo[5.2.1.0^{2,6}]decane-3,5-dione (Ishizumi 1991) (1 mmol), 1-((5-²H)pyrimidin-2-yl)piperazin (1 mmol) and 0.5 ml formalin-D₂ (20% w/w in D₂O) in 2 ml dioxane is added drop wise a solution of copper sulphate (0.05 mmol) in D₂O (1 ml) at room temperature. The mixture is then stirred at 70-80° C. for 70 minutes, cooled to room temperature and concentrated in vacuo. The product treated with toluene, filtered and concentrated in vacuo. Purification by column chromatography gives (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]decane-3,5-dione.

A solution of (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]decane-3,5-dione (0.5 mmol) in methanol (50 ml) is hydrogenated over 10% palladium on charcoal (10 mg) at 8 atm of hydrogen and 100° C. for 2 hours. The mixture is filtered and concentrated in vacuo. Purification by column chromatography gives the title compound ((1R,2R,6S,7S)-4-{4-[4-((5-²H)pyrimidin-2-yl)piperazin-1-yl]-(4-²H₂)butyl}-4-azatricyclo[5.2.1.0^{2,6}]decane-3,5-dione).

### Example 4

### (1R,2R,6S,7S)-4-{4-[4-(pyrimidin-2-yl)piperazin-1-yl]butyl}-(8,9-²H₂)-4-azatricyclo[5.2.1.0^{2,6}]decane-3,5-dione

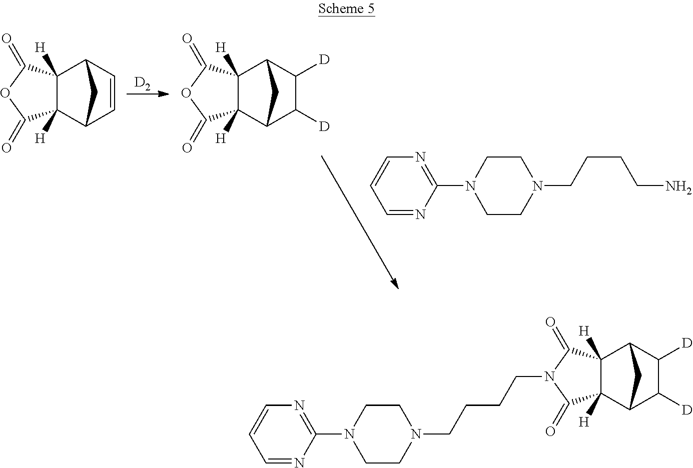

A mixture of 4-oxatricyclo[5.2.1.0^{2,6}]dec-8-ene-3,5-dione (0.1 mol) and 50% water-containing 10% palladium on charcoal (0.8 g) in tetrahydrofuran (THF) is treated with D₂ gas at room temperature. Upon filtration the mixture was concentrated in vacou to give (8,9-²H₂)-4-oxatricyclo[5.2.1.0^{2,6}]decane-3,5-dione.

A mixture of 4-[4-(pyrimidin-2-yl)piperazin-1-yl]-4-butan-1-amine (3 mmol), (8,9-²H₂)-4-oxatricyclo[5.2.1.0^{2,6}]decane-3,5-dione (5 mmol) and pyridine (15 ml) is refluxed for 5 hours. The mixture is cooled to room temperature, concentrated in vacuo and purified by column chromatography to give the title compound (1R,2R,6S,7S)-4-{4-[4-(pyrimidin-2-yl)piperazin-1-yl]butyl}-(8,9-²H₂)-4-azatricyclo[5.2.1.0^{2,6}]decane-3,5-dione).

### Example 5

### (1R,2R,6S,7S)-4-{4-[4-((5-²H)pyrimidin-2-yl)piperazin-1-yl]butyl}-(8,9-²H₂)-4-azatricyclo[5.2.1.0^{2,6}]decane-3,5-dione

Method 1:

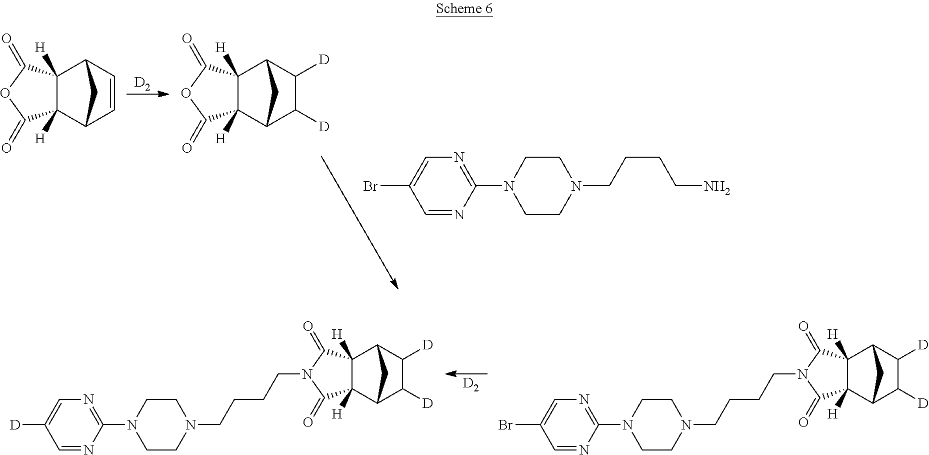

A mixture of 4-[4-(5-bromopyrimidin-2-yl)piperazin-1-yl]-4-butan-1-amine (3 mmol), (8,9-²H₂)-4-azatricyclo[5.2.1.0^{2,6}]decane-3,5-dione (5 mmol) and pyridine (15 ml) is refluxed for 5 hours. The mixture is cooled to room temperature, concentrated in vacuo and purified by column chromatography to give the title compound (1R,2R,6S,7S)-4-{4-[4-(5-bromopyrimidin-2-yl)piperazin-1-yl]butyl}-(8,9-²H₂)-4-azatricyclo[5.2.1.0^{2,6}]decane-3,5-dione).

A mixture of (1R,2R,6S,7S)-4-{4-[4-(5-bromopyrimidin-2-yl)piperazin-1-yl]butyl}-(8,9-²H₂)-4-azatricyclo[5.2.1.0^{2,6}]decane-3,5-dione (0.1 mol) and 50% water-containing 10% palladium on charcoal (0.8 g) in tetrahydrofuran (THF) is treated with D₂ gas at room temperature. Upon filtration the mixture is concentrated in vacuo to give the title compound (1R,2R,6S,7S)-4-{4-[4-((5-²H)pyrimidin-2-yl)piperazin-1-yl]butyl}-(8,9-²H₂)-4-azatricyclo[5.2.1.0^{2,6}]decane-3,5-dione).

### Method 2:

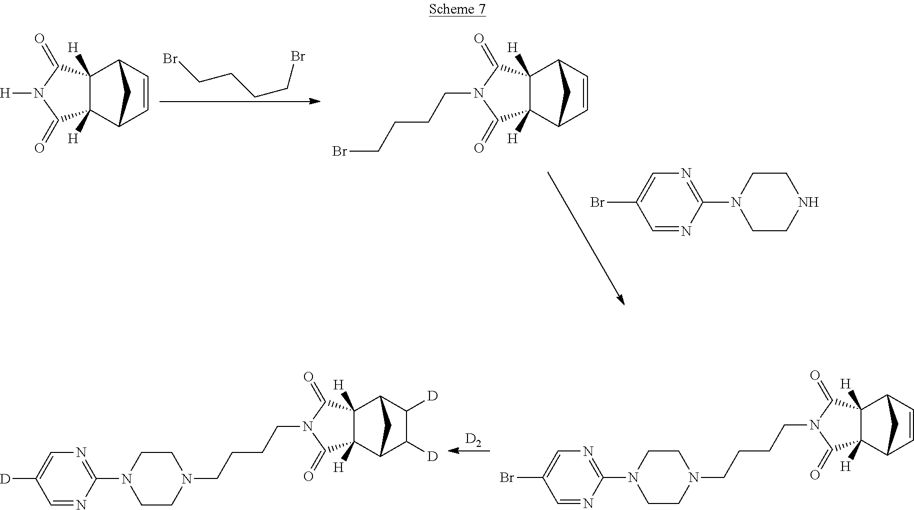

A mixture of (1R,2S,6R,7S)-4-azatricyclo[5.2.1.0^{2,6}]dec-8-ene-3,5-dione (0.15 mmol), dibromobutane (0.75 mmol) and anhydrous K₂CO₃ (0.225 mmol) in acetone is refluxed for 7 hours, cooled and filtered. The filtrated is concentrated in vacuo and the residue purified by column chromatography to give (1R,2S,6R,7S)-4-(4-bromobutyl)-4-azatricyclo[5.2.1.0^{2,6}]dec-8-ene-3,5-dione.

To a mixture of 1-(5-bromopyrimidin-2-yl)piperazin (20 mmol), anhydrous K₂CO₃ (20 mmol) and potassium iodide (2 mmol) in anhydrous DMF is added (1R,2S,6R,7S)-4-(4-bromobutyl)-4-azatricyclo[5.2.1.0^{2,6}]dec-8-ene-3,5-dione with stirring. The mixture is then stirred at 90° C. for 1 hour, cooled, poured into water and extracted with ethyl acetate. The organic phase is washed with water and brine, dried and concentrated in vacuo to give (1R,2R,6S,7S)-4-{4-[4-(5-bromopyrimidin-2-yl)piperazin-1-yl]-(4-²H₂)butyl}-(8,9-²H₂)-4-azatricyclo[5.2.1.0^{2,6}]decane-3,5-dione.

A mixture of (1R,2R,6S,7S)-4-{4-[4-(5-bromopyrimidin-2-yl)piperazin-1-yl]-(4-²H₂)butyl}-(8,9-²H₂)-4-azatricyclo[5.2.1.0^{2,6}]decane-3,5-dione (1 mmol) and 50% water-containing 10% palladium on charcoal (0.8 g) in ethanol is treated with D₂ gas at room temperature. Upon filtration the mixture is concentrated in vacuo to give the title compound (1R,2R,6S,7S)-4-{4-[4-((5-²H)pyrimidin-2-yl)piperazin-1-yl]butyl}-(8,9-²H₂)-4-azatricyclo[5.2.1.0^{2,6}]decane-3,5-dione.

### Example 6

### (1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione

### Method 1.

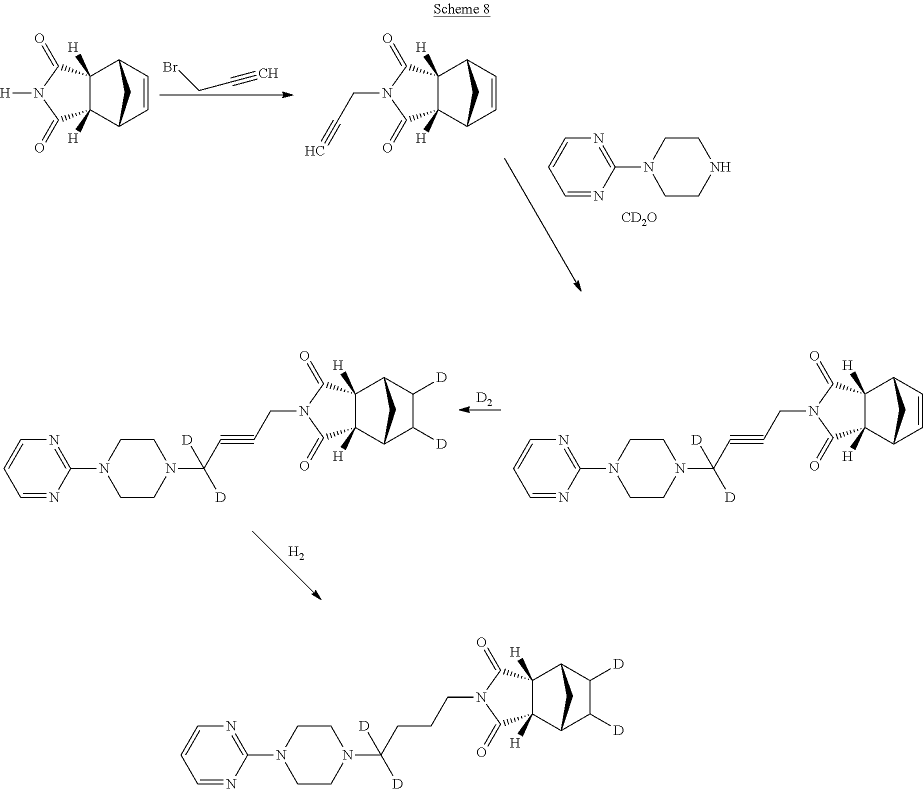

To a stirred solution of (1R,2S,6R,7S)-4-(prop-2-yn-1-yl)-4-azatricyclo[5.2.1.0^{2,6}]dec-8-ene-3,5-dione (1 mmol), 1-(pyrimidin-2-yl)piperazin (1 mmol) and 0.5 ml formalin-D₂ (20% w/w in D₂O) in 2 ml dioxane is added drop wise a solution of copper sulphate (0.05 mmol) in D₂O (1 ml) at room temperature. The mixture is then stirred at 70-80° C. for 70 minutes, cooled to room temperature and concentrated in vacuo. The product treated with toluene, filtered and concentrated in vacuo. Purification by column chromatography gives (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]dec-8-ene-3,5-dione.

A mixture of (1R,2S,6R,7S)-4-{4-[4-(pyrimidin-2-yl)piperazin-1-yl]-(4-²H₂)but-2-yn-1-yl}-4-azatricyclo[5.2.1.0^{2,6}]dec-8-ene-3,5-dione (1 mmol) and 50% water-containing 10% palladium on charcoal (0.8 g) in ethanol is treated with D₂ gas at room temperature. Upon filtration the mixture is concentrated in vacuo to give (1R,2S,6R,7S)-4-{4-[4-(pyrimidin-2-yl)piperazin-1-yl]-(4-²H₂)but-2-yn-1-yl}-(8,9-²H₂)-4-azatricyclo-[5.2.1.0^{2,6}]-decane-3,5-dione.

A solution of (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione (0.5 mmol) in methanol (50 ml) is hydrogenated over 10% palladium on charcoal (10 mg) at 8 atm of hydrogen and 100° C. for 2 hours. The mixture is filtered and concentrated in vacuo. Purification by column chromatography gives the title compound ((1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione).

### Method 2.

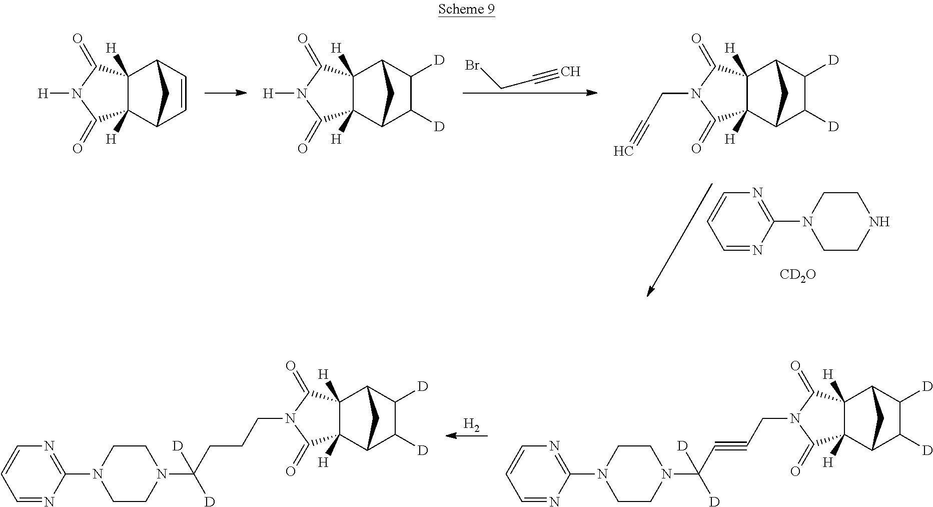

A mixture of (1R,2S,6R,7S)-4-azatricyclo[5.2.1.0^{2,6}]dec-8-ene-3,5-dione (0.1 mol) and 50% water-containing 10% palladium on charcoal (0.8 g) in tetrahydrofuran (THF) is treated with D₂ gas at room temperature. Upon filtration the mixture was concentrated in vacou to give (8,9-²H₂)-4-azatricyclo[5.2.1.0^{2,6}]decane-3,5-dione.

A mixture of (8,9-²H₂)-4-azatricyclo[5.2.1.0^{2,6}]decane-3,5-dione (0.1 mmol) and propagyl bromide (0.12 mmol) and anhydrous K₂CO₃ (0.14 mmol) in anhydrous acetone (10 ml) is refluxed under nitrogen for 1 hour, cooled and filtered. The filtrate is concentrated in vacuo and purified by recrystallization from n-hexane to give (1R,2S, 6R,7S)-4-(prop-2-yn-1-yl)-4(8,9-²H₂)-4-azatricyclo[5.2.1.0^{2,6}]decane-3,5-dione.

To a stirred solution of (1R,2S,6R,7S)-4-(prop-2-yn-1-yl)-4(8,9-²H₂)-4-azatricyclo[5.2.1.0^{2,6}]decane-3,5-dione (1 mmol), 1-(pyrimidin-2-yl)piperazin (1 mmol) and 0.5 ml formalin-D₂ (20% w/w in D₂O) in 2 ml dioxane is added drop wise a solution of copper sulphate (0.05 mmol) in D₂O (1 ml) at room temperature. The mixture is then stirred at 70-80° C. for 70 minutes, cooled to room temperature and concentrated in vacuo. The product treated with toluene, filtered and concentrated in vacuo. Purification by column chromatography gives (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]dec-8-ene-3,5-dione.

A solution of (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione (0.5 mmol) in methanol (50 ml) is hydrogenated over 10% palladium on charcoal (10 mg) at 8 atm of hydrogen and 100° C. for 2 hours. The mixture is filtered and concentrated in vacuo. Purification by column chromatography gives the title compound ((1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione).

### Example 7

### (1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione

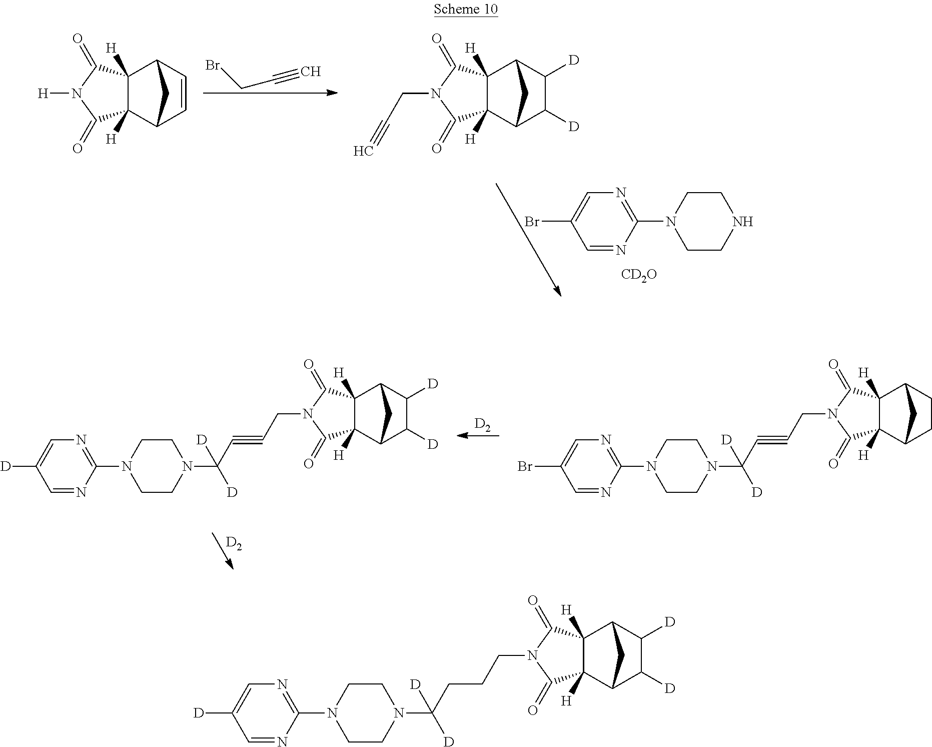

To a stirred solution of (1R,2S,6R,7S)-4-(prop-2-yn-1-yl)-4-azatricyclo[5.2.1.0^{2,6}]dec-8-ene-3,5-dione (1 mmol), 1-((5-bromopyrimidin-2-yl)piperazin (1 mmol) and 0.5 ml formalin-D₂ (20% w/w in D₂O) in 2 ml dioxane is added drop wise a solution of copper sulphate (0.05 mmol) in D₂O (1 ml) at room temperature. The mixture is then stirred at 70-80° C. for 70 minutes, cooled to room temperature and concentrated in vacuo. The product treated with toluene, filtered and concentrated in vacuo. Purification by column chromatography gives (1R,2S,6R,7S)-4-{4-[4-(5-bromopyrimidin-2-yl)piperazin-1-yl]-(4-²H₂)but-2-yn-1-yl}-4-azatricyclo[5.2.1.0^{2,6}]dec-8-ene-3,5-dione.

A mixture of (1R,2S,6R,7S)-4-{4-[4-(5-bromopyrimidin-2-yl)piperazin-1-yl]-(4-²H₂)but-2-yn-1-yl}-4-azatricyclo[5.2.1.0^{2,6}]dec-8-ene-3,5-dione (1 mmol) and 50% water-containing 10% palladium on charcoal (0.8 g) in ethanol is treated with D₂ gas at room temperature. Upon filtration the mixture is concentrated in vacuo to give (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione.

A solution of (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione (0.5 mmol) in methanol (50 ml) is hydrogenated over 10% palladium on charcoal (10 mg) at 8 atm of hydrogen and 100° C. for 2 hours. The mixture is filtered and concentrated in vacuo. Purification by column chromatography gives the title compound ((1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione).

### Example 8

### Pharmacological Methods

Metabolic stability of compounds of the present invention can be evaluate by procedures know to persons skilled in the art. Several examples of such procedures can be found in Methling et al: Drug Metabolism and Disposition (2009), 37:479-493.

### Pharmacological Methods for Determining Pharmacokinetic Properties

A person skilled in the art will recognize that commercially available apparatus mentioned as part of the methods below are interchangeable with comparative apparatus for similar purposes obtained through other producers or vendors.

Degradation by CYP450 Isoenzymes Incubation with recombinant human CYP isoforms (i.e. CYP2D6 CYP3A4, CYP2C9, CYP2C19 and CYP1A2) is performed as described in the art, (for instance as described by Suzuki et al. 1999), in brief:

The basic incubation medium contained 10 μmol/ml, 15 mM MgCl₂, 1.3 mM NADP, 3.3 mM glucose 6-phosphate, 0.4 I.U./ml glucose 6-phosphate dehydrogenase, 100 mM potassium phosphate buffer (pH 7.4), 0.1 mM EDTA, and 1 μM of compounds of the present invention, in a final volume of 200 ml. The mixture is incubated at 37° C. in a shaking water bath for 60 min. The reaction is terminated by addition of 10 ml of perchloric acid and 50 ml of a methanolic solution of the internal standard. After termination of the incubation, the mixtures are centrifuged at 10,000 rpm for 1 min, and the supernatants are analyzed by HPLC-MS/MS.

Stability in Rat Liver Microsomes: Incubations of Compounds or Pharmacological Compositions with Rat Liver Microsomes and S9

Microsomes from non-induced and from dexamethasone-, rifampicin-, phenobarbital-, and β-naphthoflavone-induced rat livers are used (Walter et al. 2003). The NADPH-regenerating system and NADPH negative controls are incubated for 5 min at 37° C. in open tubes. Next, the required volume of the substrates is added, and the solutions mixed and divided into 2-ml reaction vials. The reactions are started by the addition of the microsomal suspension to give a final concentration of 2 mg/ml protein (2-8 mg/ml protein for S9). The final volume of each incubation mixture is 1 ml containing 20 μM substrate, 0.5 mM NADP⁺, 5 mM glucose 6-phosphate, 10 mM MgCl₂.6H₂O, 5 mM EDTA, and 3.5 IU/ml glucose-6-phosphate dehydrogenase. Incubations are continued at 37° C. At the appropriate times (0, 15, and 30 min) 180 μl is removed and added to 180 μl of ice-cold acetonitrile. After mixing, the samples are placed on ice for 30 min to facilitate protein precipitation. Finally, the samples are centrifuged at 14,000 g at 0° C. for 10 min. The centrifuged samples are stored at −32° C. Immediately before HPLC analysis the samples are mixed and centrifuged once more. The supernatant are analyzed by quantitative HPLC analysis method.

### HPLC/HRMS Analysis

All chromatographic separations for HRMS measurements and the isolation of metabolites are done with a HPLC system (e.g. Agilent 1100) consisting of a quaternary gradient pump, an autosampler, and a solvent degasser. The column is connected to the BNMI-HP unit for beam splitting (20:1) followed by the Bruker diode array UV detector (Bruker BioSpin GmbH, Rheinstetten, Germany) in parallel with the micrOTOF mass spectrometer (Bruker Daltonics, Bremen, Germany). The micrOTOF mass spectrometer is equipped with an electrospray ion source (temperature 180° C.).

Mass spectra are acquired with a scan range from 50 to 1500 m/z. All measurements are done in the positive mode. For all separations, a 125×4 mm LiChrospher 100 RP-18e (5 μm) column (Merck, Darmstadt, Germany) preceded by a pre-column of the same material are used. The flow rate is 0.5 ml/min. The chromatography is performed at 23±2° C. Detection is done at λ=204, 247, and 319 nm (maxima of absorption) and 362 nm (minimum of absorption) for analytes. Metabolite fractions for MS/MS analysis with an API 4000 mass spectrometer is collected manually. Eluents used in the gradients are acetonitrile (solvent B) and 50 mM ammonium acetate adjusted to pH 7.5 with 2.5% ammonia (solvent D). Solvent gradients for all chromatographic separations run from 10 to 100% solvent B in 25 min, with the shapes of the gradients optimized for separations. These methods are used in the analysis of incubations of tandospirone in the presence of microsomes with UDP-GA or in the presence of HRP and H₂O₂ with GSH.

### HPLC/MS/MS Analysis

The MS/MS analysis of metabolites of compounds of the invention can be done using the following procedure. The equipment can consist of an Agilent gradient pump 1100, a column oven, an autosampler, and a linear ion trap quadrupole mass spectrometer 3200 Q TRAP (Applied Biosystems/MDS Sciex, Foster City, Calif.). The source type is Turbo Spray with a source temperature of 450° C. For all measurements the positive mode is used. A Phenomenex Synergi Hydro RP column, 150×2 mm (4 μm), is used for the chromatography with a flow rate of 0.3 ml/min. Separations are performed using 95% A and 5% B for 30 s as a gradient, followed by a linear increase to 100% B over 15.5 min and then by 2 min of 100% B. Afterward the column is reconstituted to the starting conditions over 7 min. Solvent A used in the gradient is 5 mM ammonium acetate and solvent B is methanol containing 5 mM ammonium acetate. The column is heated to 30° C.

### MS/MS Analysis

MS/MS analyses metabolites of compounds of the present invention also could be done with an API 4000 mass spectrometer (AB/MDS Sciex). Purified metabolite fractions are analyzed by flow injection analysis by using a solvent flow of acetonitrile-50 mM ammonium acetate buffer (pH=7.5) (solvent ratios resulting from the further separations) at flow rates of 10 and 20 μl/min, respectively. The mass spectrometer is equipped with an electrospray ion source (temperature 300° C.). Collision-induced dissociation (CID) spectra are acquired for all metabolites with nitrogen as the collision gas. Collision energies used are in a range between 20 and 65 eV.

Incubations of Compounds with Human Liver Microsomes and S9

Incubations are done under the same conditions as described for rat liver microsomes. Modifications are as follows: final protein concentration is 4 mg/ml for S9; final volume of each incubation mixture is 320 μl; and sample volume 80 μl.

### Stability in Human Hepatocytes

In vitro stability in the presence of hepatocytes is conducted as follows. Fresh or cryopreserved hepatocytes are thawed if necessary, isolated from shipping media and diluted to a viable cell density of 2×106 cells/mL according to the supplier's guidelines using Krebs-Henseleit buffer (KHB, pH 7.3, Sigma) supplemented with amikacin (84 μg/mL), calcium chloride (1 mM), gentamicin (84 μg/mL), HEPES (20 mM), heptanoic acid (4.2 μM) and sodium bicarbonate (2.2 mg/mL). Viability is determined by trypan blue exclusion using a hemacytometer (3500 Hausser, VWR). A 10-mM DMSO stock solution of each drug is diluted to 2 μM using supplemented KHB buffer to create the working standard. A 50-μL aliquot of test compound or control are added to each test well of a 96-well polypropylene plate (Costar) immediately followed by the addition of 50 μL of the hepatocyte suspension. One incubation plate is prepared for each timepoint (i.e., 0, 30, 60, and 120 minutes) with samples being prepared in duplicate.

For these determinations, experiments are conducted in triplicate. Incubations are conducted at 37° C., 5% CO₂ and 100% relative humidity in an incubator (Model 2300, VWR). At each timepoint, one incubation plate is removed from the incubator, and a solution containing internal standard (100 μL, 2 μM labetalol) is added to each well. The plate is mixed at 700 rpm for 2 minutes on a plate shaker (IKA MTS 2/4 Digital Microtiter Shaker, VWR) and immediately centrifuged at 2,000×g for 10 minutes using an Allegra benchtop centrifuge (Beckman Coulter). A 150-μL aliquot of the supernatant is transferred from each well to a 96-well shallow plate (Costar). The plates are sealed using reusable plate mats. Quantitation is performed using an ion trap LC-MS/MS method (Finnigan). Chromatographic separation is achieved using a YMC ODS AQ C18 column (2.1×30 mm, 3 μm, 180 Å) in conjunction with a 6-minute gradient using mobile phases A (aqueous 0.1% formic acid containing 1% isopropanol) and B (0.1% formic acid in acetonitrile containing 1% isopropanol). Mass spectrometric detection of the analytes is accomplished using ESI+ or APCI+ ionization modes. Analyte responses are measured using extracted ion chromatograms of characteristic fragments from the [M+H]⁺ ion. Calculations are performed using Excel (Microsoft).

### Pharmacokinetic and Bioavailability Analysis of Tandospirone Compounds Following Oral and Intravenous Administration to Rats

Three male Sprague-Dawley rats (200-250 g each) are cannulated in the jugular vein and administered a single dose containing 2 mg/kg each of the compounds of the present invention. Three additional male Sprague-Dawley rats (200-250 g each) are administered a single dose containing 2 mg/kg of the compounds of the present invention by oral gavage.

Blood (0.25 ml) from intravenously treated rats is collected retro-orbitally at 2, 5, 15, and 30 minutes, and 1, 2, 4, and 6 hours post-dosing. Blood (0.25 ml) from orally treated rats is collected retro-orbitally at 5, 15, 30, and 45 minutes and 1, 2, 4, and 6 hours post-dosing. Blood is collected into tubes containing K₂EDTA as coagulant at the above mentioned time points. Blood samples are stored on ice and then centrifuged to obtain plasma. The plasma (about 0.125 μl) is aliquoted into 96-well plats and stored at −80° C. until analysis by LC-MS/MS for example using an Applied Bio-system API 4000 mass spectrometer.

Metabolic stability of compounds of the present invention can also be evaluated by other procedures know to persons skilled in the art. Several examples of such procedures can be found in e.g. Konsoula et al: Int J. Pharm. 2008 September 1; 361(1-2): 19-25 and Methling et al.

### Stability in Human Plasma

The plasma is diluted to 80% with 0.05 M PBS (pH 7.4) at 37° C. The reactions are initiated by the addition of the compounds to 1 ml of preheated plasma solution to yield a final concentration of 200 μM. The assays are performed in a shaking water bath at 37° C. and conducted in triplicate. Samples (50 μl) were taken at 0, 15, 30, 45, 60, 90 min and added to 200 μl acetonitrile in order to deproteinize the plasma. The samples are subjected to vortex mixing for 1 min and then centrifugation at 4° C. for 15 min at 14,000 rpm. The clear supernatants are analyzed by HPLC. The values represent the mean of three independent experiments. The in vitro plasma half life (t½) is calculated using the expression t_{1/2}=0.693/b, where b is the slope found in the linear fit of the natural logarithm of the fraction remaining of the parent compound vs. incubation time.

### Example 9

### Efficacy in Patients Suffering from Atopic Dermatitis

### Measuring Effects of Treatment

Patients suffering from atopical dermatitis as determined by the SCORAD Index are selected for the study. If patients are already using other agents for treatment of atopical dermatitis such as oral antihistamines or topical corticosteroids, they are eventually allowed to continue with them. The deuterated tandospirone derivatives, which are formulated in a way that will make them suitable for oral use, are given to the patients 1-3 times daily for a period of 4 weeks. The severity of the disease is assessed before the first treatment and at days 15 and 29 of the treatment period by the investigating physician using the SCORAD index. Itching is rated twice daily by the patients themselves using a visual analogue scale (VAS).

The efficacy of the treatment is determined using the SCORAD index and the VAS in comparison to the pre-treatment values and eventually to a group of non-treated patients, to placebo treated patients or to a group of patients treated with other medications.

### Childhood Atopic Dermatitis

A 2 year old child has developed itching and rashes covering various parts of the body including the arms, legs and neck and specifically the elbows and knees.

The child is diagnosed with atopic dermatitis, and is treated with deuterated tandospirone tablets of 10 mg of given 2 times daily for a period of 4 weeks.

During this treatment period is observed a visual effect on the rashes, and an improved sleeping pattern. The efficacy of the treatment is determined using the SCORAD index which decreases significantly during the treatment.

### Adult Dermatitis

A 25 years old man working as a chef is diagnosed with undefined dermatitis and has symptoms of severe itching, dry, rough and scaly skin patches and cracked skin on the hands.

The patient is treated with a combination of a steroid ointment applied topically 2 times a day and tablets of deuterated tandospirone 20 mg given 2 times daily for a period of 4 weeks.

During this treatment period is observed a visual effect on the rashes which are significantly reduced in size, and the cracks in the skin are healed.

### Example 10

### Synthesis of (1R,2R,6S,7S)-4-{4-[4-((5-²H)pyrimidin-2-yl)piperazin-1-yl]butyl}-4-azatricyclo[5.2.1.0^{2,6}]decane-3,5-dione

### Method 3

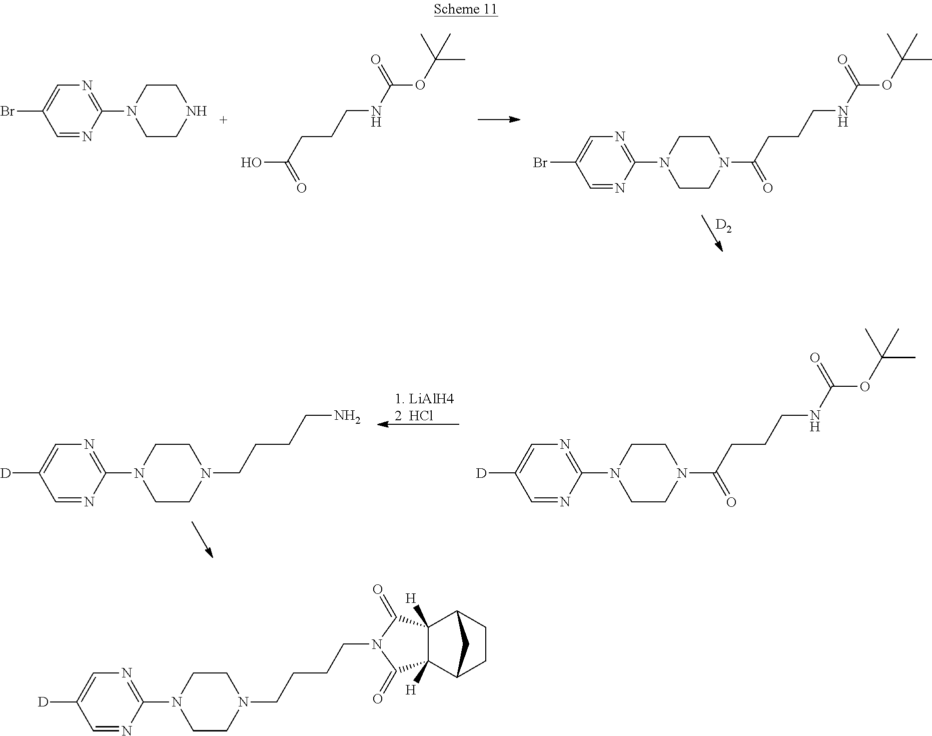

### Preparation of tert-butyl 4-oxo-4-(4-(5-bromopyrimidin-2-yl)piperazin-1-yl)butylcarbamate

A solution of 4-(tert-butoxycarbonylamino)butanoic acid (0.609 g, 3 mmol) in dichloromethane (DCM) (20 ml) is added 2-(1H-7-azabenzotriazol-1-yl)-1,1,3,3-tetramethyl uronium hexafluorophosphate methanaminium (HATU) (2.28 g, 6 mmol) and Et₃N (1.66 ml, 12 mmol). The mixture is stirred for 0.5 h and 2-(piperazin-1-yl)-(5-bromo)pyrimidine (3 mmol) is added. The resulting mixture is stirred at room temperature overnight. The reaction mixture is concentrated in vacuo. The residue is purified by chromatography on silica gel (100% EtOAc) to afford tert-butyl 4-oxo-4-(4-(5-bromo)(pyrimidin-2-yl)piperazin-1-yl)butylcarbamate as a clear oil, which solidifies while standing at room temperature.

### Preparation of tert-butyl 4-oxo-4-(4-(5-2H)pyrimidin-2-yl)piperazin-1-yl)butylcarbamate

A mixture of tert-butyl 4-oxo-4-(4-(pyrimidin-2-yl)piperazin-1-yl)butylcarbamate (0.1 mol) and 50% water-containing 10% palladium on charcoal (0.8 g) in tetrahydrofuran mol) and 50% water-containing 10% palladium on charcoal (0.8 g) in tetrahydrofuran (THF) is treated with D₂ gas at room temperature. Upon filtration the mixture is concentrated in vacuo to give tert-butyl 4-oxo-4-(4-(5-²H)pyrimidin-2-yl)piperazin-1-yl)butylcarbamate.

### Preparation of 4-(4-(5-²H)pyrimidin-2-yl)piperazin-1-yl) butan-1-amine, hydrochloride

A solution of tert-butyl 4-oxo-4-(4-(5-(H²)pyrimidin-2-yl)piperazin-1-yl)butylcarbamate (10 mmol) in methanol (50 ml) is treated with 3.0 M HCl/MeOH (50 ml, 250 mmol) for 3 hrs. The reaction mixture is concentrated in vacuo to give 4-(4-(5-²H)pyrimidin-2-yl)piperazin-1-yl) butan-1-amine, hydrochloride

### Preparation of (1R,2R,6S,7S)-4-{4-[4-((5-²H)pyrimidin-2-yl)piperazin-1-yl]butyl}-4-azatricyclo[5.2.1.02,6]decane-3,5-dione

A solution of bicyclo[2.2.1]heptane-2,3-dicarboxylic anhydride (4 mmol) and 4-(4-(5-²H)pyrimidin-2-yl)piperazin-1-yl)butan-1-amine, hydrochloride (4.8 mmol) in pyridine (15 ml) is heated at reflux for an hour. The reaction mixture was concentrated in vacuo to remove the solvent. The residue is purified by column chromatography and (1R,2R,6S,7S)-4-{4-[4-((5-²H)pyrimidin-2-yl)piperazin-1-yl]butyl}-4-azatricyclo[5.2.1.02,6]decane-3,5-dione.

### Preparation of tert-butyl 4-oxo-4-(4-(5-bromopyrimidin-2-yl)piperazin-1-yl)butylcarbamate

A solution of 4-(tert-butoxycarbonylamino)butanoic acid (0.609 g, 3 mmol) in dichloromethane (DCM) (20 ml) is added 2-(1H-7-azabenzotriazol-1-yl)-1,1,3,3-tetramethyl uronium hexafluorophosphate methanaminium (HATU) (2.28 g, 6 mmol) and Et₃N (1.66 ml, 12 mmol). The mixture is stirred for 0.5 h and 2-(piperazin-1-yl)-(5-bromo)pyrimidine (3 mmol) is added. The resulting mixture is stirred at room temperature overnight. The reaction mixture is concentrated in vacuo. The residue is purified by chromatography on silica gel (100% EtOAc) to afford tert-butyl 4-oxo-4-(4-(pyrimidin-2-yl)piperazin-1-yl)butylcarbamate as a clear oil, which solidifies while standing at room temperature.

### Example 11

### Synthesis of (1R,2R,6S,7S)-4-{4-[4-(pyrimidin-2-yl)piperazin-1-yl]-(4,4-²H₂)butyl}-4-azatricyclo[5.2.1.0^{2,6}]decane-3,5-dione

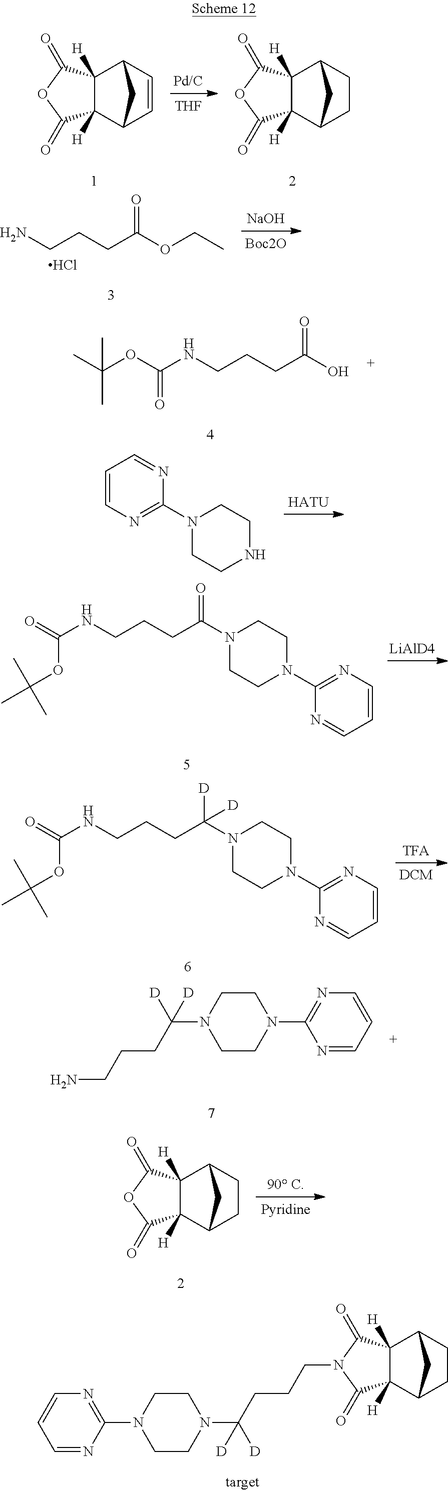

### Preparation of bicyclo[2.2.1]heptane-2,3-endo-dicarboxylic anhydride (Intermediate 2)

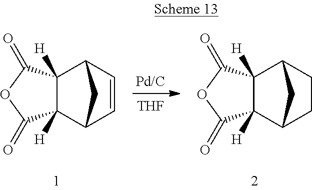 [SMILES: O=C1OC(=O)[C@H]2[C@@H]1[C@H]1C=C[C@@H]2C1 ; O=C1OC(=O)[C@H]2[C@H]3CC[C@H](C3)[C@@H]12 ; "THF"]

A solution of starting compound bicyclo[2.2.1]hept-5-ene-2,3-endo-dicarboxylic anhydride (0.82 g, 5 mmol) in THF (30 ml) was added Pd/C (10%, 0.53 g, 0.5 mmol). The mixture was stirred at room temperature under H₂ for 16 hours. The mixture was filtered. The filtrate was concentrated under reduced pressure to give intermediate bicyclo[2.2.1]heptane-2,3-endo-dicarboxylic anhydride (0.82 g, 4.9 mmol, 99% yield) as white solid.

Melting point: 163.8˜164.6° C.

¹HNMR (400 MHz, CDCl3): δ 3.41 (s, 2H), 2.86 (s, 2H), 1.69-1.73 (m, 4H), 1.45-1.51 (m, 2H)

### Preparation of 4-(tert-butoxycarbonylamino)butanoic acid (Intermediate 4)

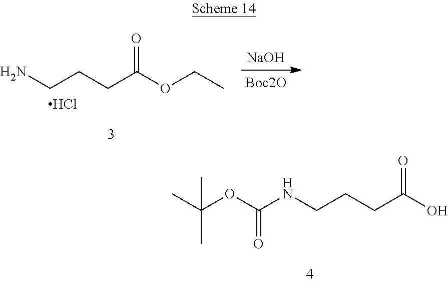

Ethyl 4-aminobutanoate hydrochloride (1.68 g, 10 mmol) was added to the solution of NaOH (1.2 g, 30 mmol) in H₂O (20 ml). The mixture was heated at 80° C. for 4 hours. After the reaction mixture was cooled to room temperature, Boc₂O (2.3 g, 10.5 mmol) in 1,4-dioxane (10 ml) was added. The mixture was stirred for another 4 hours. The reaction mixture was concentrated under reduced pressure. The residue was adjusted to pH=3 with dilute HCl solution, and then extracted with EtOAc (10 ml*4). The combined EtOAc was washed with brine (20 ml), dried over Na₂SO₄, and concentrated in vacuo to afford 4-(tert-butoxycarbonylamino)butanoic acid (1.6 g, 67%) as a clear oil, which solidified while standing at room temperature.

Melting point: 36.9˜56° C.

LCMS: RT=1.37 min, m/z=226 (M+Na)

¹HNMR (400 MHz, DMSO-d6): δ 12.05 (s, 1H), 6.85 (m, 1H), 2.92 (m, 2H), 2.19 (m, 2H), 1.58 (m, 2H), 1.38 (s, 9H).

### Preparation of tert-butyl 4-oxo-4-(4-(pyrimidin-2-yl)piperazin-1-yl) butylcarba-mate (Intermediate 5)

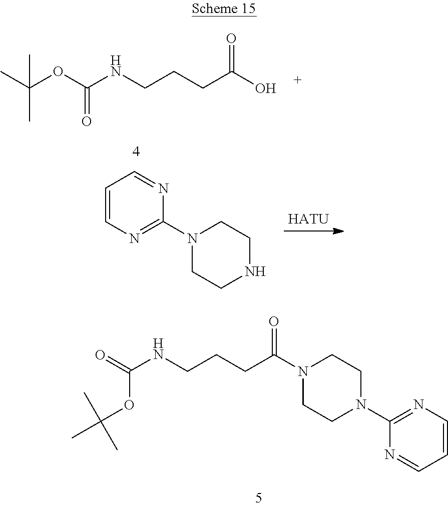

A solution of 4-(tert-butoxycarbonylamino)butanoic acid (0.609 g, 3 mmol) in DCM (20 ml) was added HATU (2.28 g, 6 mmol) and Et₃N (1.66 ml, 12 mmol). After the mixture was stirred for 0.5 h, 2-(piperazin-1-yl)pyrimidine (0.49 g, 3 mmol) was added. The resulting mixture was stirred at room temperature overnight. The reaction mixture was concentrated in vacuo. The residue was purified by chromatography on silica gel (100% EtOAc) to afford tert-butyl 4-oxo-4-(4-(pyrimidin-2-yl)piperazin-1-yl) butylcarbamate (1.6 g, 97% yield) as a clear oil, which solidified while standing at room temperature.

Melting point: 106.2˜124.4° C.

LCMS: RT=1.42 min, m/z=350.1 (M+1).

¹HNMR (400 MHz, CDCl3): δ 8.39 (d, 2H), 6.84 (m, 1H), 6.68 (m, 1H), 3.76 (m, 4H); 3.52 (m, 4H), 2.96 (m, 2H), 2.37 (M, 2H), 1.64 (m, 2H), 1.37 (s, 9H)

### Preparation of tert-butyl 4-(4-(pyrimidin-2-yl)piperazin-1-yl) (4,4-²H₂)-butylcarbamate (Intermediate 6)

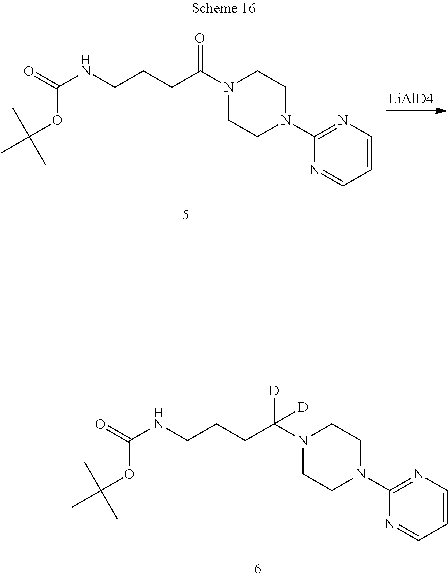 [SMILES: CC(C)(C)OC(=O)NCCCC(=O)N1CCN(c2ncccn2)CC1 ; "LiAlD4" ; [2H]C([2H])(CCCNC(=O)OC(C)(C)C)N1CCN(c2ncccn2)CC1]

A solution of tert-butyl 4-oxo-4-(4-(pyrimidin-2-yl)piperazin-1-yl) butylcarbamate (1 g, 2.9 mmol) in THF (30 ml) was added LiAlD₄ (0.13 g, 3.1 mmol) at 0° C. The resulting mixture was stirred at room temperature overnight. The reaction mixture was added Na₂SO₄.10H₂O (1.3 g) in portions. After the mixture was stirred for one hour, it was filtered to remove the solid. The filtrate was concentrated in vacuo to give tert-butyl 4-(4-(pyrimidin-2-yl)piperazin-1-yl) (4,4-²H₂)-butylcarbamate (0.94 g, 96% yield) as a colorless oil, which was used in next step without further purification.

LCMS: RT=1.48 min, m/z=338.1 (M+1)

### Preparation of 4-[4-(pyrimidin-2-yl)piperazin-1-yl]-(4,4-2H₂)— butan-1-amine (Intermediate 7)

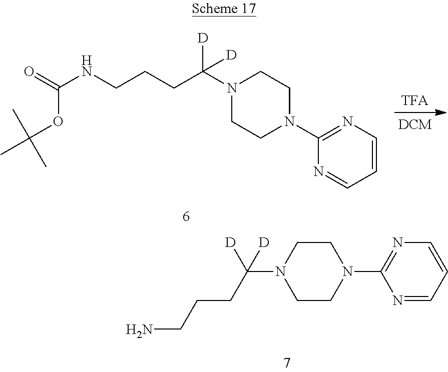

A solution of tert-butyl 4-(4-(pyrimidin-2-yl)piperazin-1-yl) (4,4-²H₂)-butylcarbamate (940 mg, 2.8 mmol) in DCM (16 ml) was added TFA (12 ml). The mixture was stirred at room temperature for 3 hrs, and then concentrated in vacuo to give 4-[4-(pyrimidin-2-yl)piperazin-1-yl]-(4,4-²H₂)— butan-1-amine as TFA salt form (1.2 g, 100% yield) as brown oil, which was used in next step without further purification.

LCMS: RT=1.08 min, m/z=238.2 (M+1).

### Preparation of (1R,2S,6R,7S)-4-{4-[4-(pyrimidin-2-yl)piperazin-1-yl]-(4,4-²H₂)but-2-yn-1-yl}-4-azatricyclo[5.2.1.0^{2,6}]decane-3,5-dione

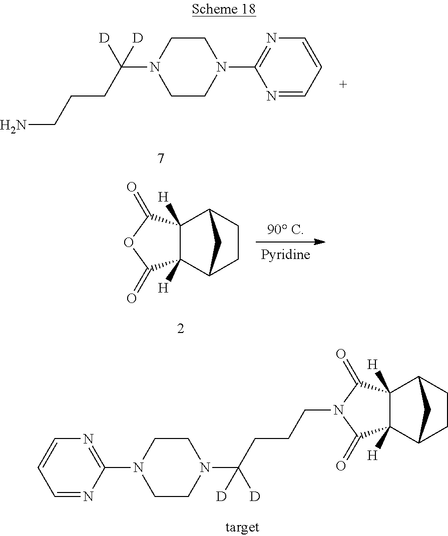 [SMILES: [2H]C([2H])(CCCN)N1CCN(c2ncccn2)CC1 ; O=C1OC(=O)[C@H]2[C@H]3CC[C@H](C3)[C@@H]12 ; "Pyridine" ; [2H]C([2H])(CCCN1C(=O)[C@@H]2[C@@H]3CC[C@@H](C3)[C@@H]2C1=O)N1CCN(c2ncccn2)CC1]

The crude 4-[4-(pyrimidin-2-yl)piperazin-1-yl]-(4,4-²H₂)— butan-1-amine as TFA salt form (1.2 g, 2.8 mmol) was added pyridine (10 ml). The solution was added intermediate 2 (bicyclo[2.2.1]heptane-2,3-endo-dicarboxylic anhydride) (0.47 g, 2.8 mmol) whereupon the mixture was heated in 90° C. oil bath for 4 hours. The reaction mixture was concentrated in vacuo to remove the solvent. The crude product was purified by chromatography on silica gel (30 g, DCM:MeOH=50:1,500 ml; 30:1, 500 ml; 25:1, 1.0 L) to give the expected product, which was purified further by prep-HPLC to give the pure tittle compound (220 mg, 20% yield) as colourless oil.

LCMS: RT=1.55 min, m/z=386.2 (M+1)

HPLC: RT=4.579 min, 100% (214 nm, 254 nm)

¹HNMR (400 MHz, CDCl3): δ 8.32 (d, 2H), 6.49 (t, 1H), 4.85 (m, 3H), 3.85 (br, 4H), 3.51 (t, 2H), 3.08 (s, 2H), 2.78 (s, 2H), 2.50 (br, 4H), 1.56˜1.67 (m, 7H), 1.25 (m, 3H).

### Preparation of (1R,2S,6R,7S)-4-{4-[4-(pyrimidin-2-yl)piperazin-1-yl]-(4,4-²H₂)but-2-yn-1-yl}-4-azatricyclo[5.2.1.0^{2,6}]decane-3,5-dione citrate

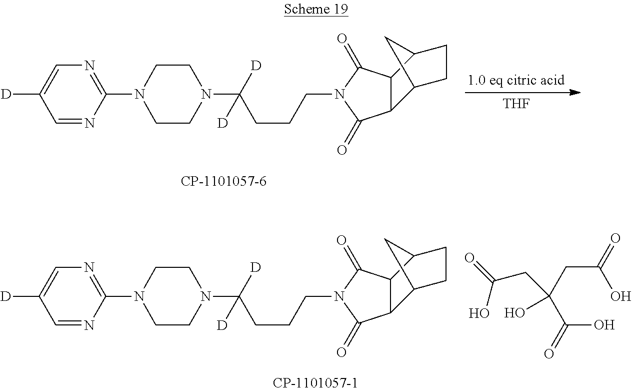

A solution of (1R,2S,6R,7S)-4-{4-[4-(pyrimidin-2-yl)piperazin-1-yl]-(4,4-²H₂)but-2-yn-1-yl}-4-azatricyclo[5.2.1.0^{2,6}]decane-3,5-dione (280 mg, 0.725 mol) and citric acid (152.4 mg, 0.725 mol) in THF (50 ml) was stirred at room temperature for 2 hours, and then the reaction solution was let to stay for 2 days. The solid was separated by filtration. The collected solid was re-dissolved in water and then dried over by lyophilization.

Mp: 60.5˜68.1° C.

LCMS: RT=1.041 min, m/z=387.1 (M+1)

¹HNMR (400 MHz, CD3OD): δ 8.41 (s, 2H); 4.11 (m, 4H); 3.53 (t, 2H); 3.26 (m, 4H); 3.18 (m, 2H); 2.84˜2.89 (d, 2H); 2.74˜2.78 (d, 2H); 2.71 (m, 2 h); 1.59˜1.81 (m, 8H); 1.20 (m, 2H)

### Example 12

### Synthesis of (1R,2R,6S,7S)-4-{4-[4-((5-²H)pyrimidin-2-yl)piperazin-1-yl]-(4-²H₂)butyl}-4-azatricyclo[5.2.1.0^{2,6}]decane-3,5-dione

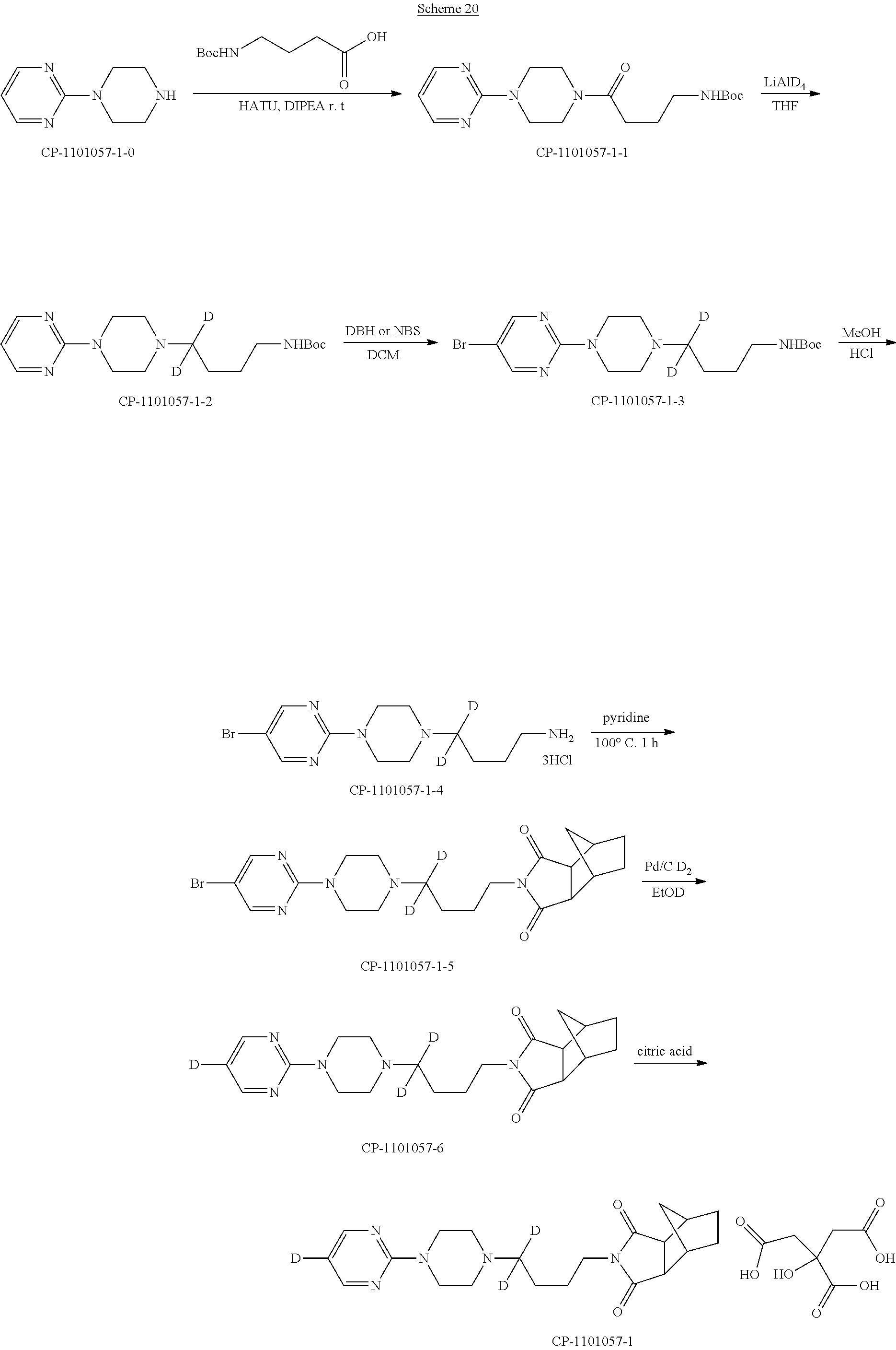

### Preparation of ethyl 4-(tert-butoxycarbonylamino)butanoate

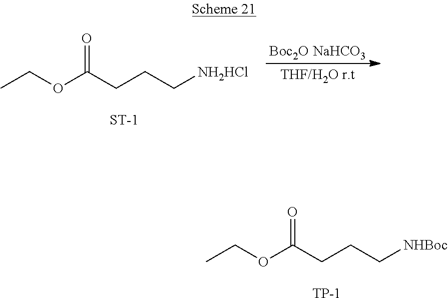

A solution of ethyl 4-aminobutanoate hydrochloride (3.0 g, 18 mmol), Boc₂O (3.91 g, 18 mmol) and K₂CO₃ (7.5 g, 54 mmol) in THF/H₂O (50 ml, 1:1) was stirred at room temperature for 4 hrs. The reaction mixture was diluted with water (100 ml), extracted with ethyl acetate (100 ml×2), the combined organic phase was washed with brine, dried over Na₂SO₄, concentrated in vacuo to give ethyl 4-(tert-butoxycarbonylamino) butanoate (3.6 g, 86.6% yield) as a white oil.

LCMS: RT=1.304 min, m/z=118 (M−100)

### Preparation of 4-(tert-butoxycarbonylamino)butanoic acid

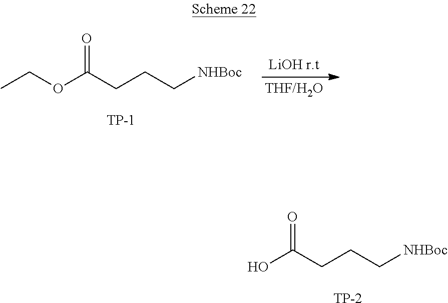

A suspension of ethyl 4-(tert-butoxycarbonylamino)butanoate (3.6 g, 15.58 mmol) and LiOHH₂O (1.96 g, 46.74 mmol) in THF/H₂O (60 ml, 1:1) was stirred at room temperature for 4 hrs. The reaction mixture was diluted with water (150 ml), extracted with Et₂O (100 ml×2). The aqueous was acidified with 2.0 N HCl to PH 3-4, and then extracted with ethyl acetate (150 ml×2), the combined organic phase was dried over Na₂SO₄, concentrated in vacuo to afford 4-(tert-butoxycarbonylamino)butanoic acid (4.5 g, 100%) as a white oil.

LCMS: RT=1.376 min, m/z=226 (M+Na)

HNMR (400 MHz, CDCl3): δ: 12.05 (s, 1H); 6.85 (br, 1H); 2.94 (m, 2H); 2.21 (m, 2H); 1.60 (m, 2H); 1.38 (s, 9H).

### Preparation of tert-butyl 4-oxo-4-(4-(pyrimidin-2-yl)piperazin-1-yl) butylcarbamate

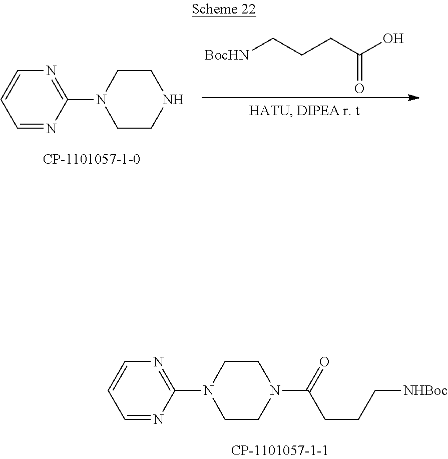 [SMILES: c1cnc(N2CCNCC2)nc1 ; CC(C)(C)OC(=O)NCCCC(=O)O ; "HATU, DIPEA r. t" ; CC(C)(C)OC(=O)NCCCC(=O)N1CCN(c2ncccn2)CC1]

A solution of 4-(tert-butoxycarbonylamino)butanoic acid (10.4 g, 21.5 mmol), HATU (10.4 g, 27.4 mmol) and DIPEA (9.54 ml, 54.9 mmol) in DCM (100 ml) was stirred at room temperature for 10 min, and then 2-(piperazin-1-yl)pyrimidine (3.0 g, 18.6 mmol) was added. The resulting mixture was stirred at room temperature for overnight. The reaction mixture was diluted with DCM (100 ml), washed with water (150 ml×2). The organic phase was dried over Na₂SO₄, concentrated in vacuo to give the crude product which was purified by combiflash column (100% EtOAc) to afford tert-butyl 4-oxo-4-(4-(pyrimidin-2-yl)piperazin-1-yl) butylcarbamate (6.43 g, ˜100% yield) as a white oil.

LCMS: RT=1.23 min, m/z=350.1 (M+1).

HNMR (400 MHz, DMSO-d6): δ: 8.39 (d, 2H); 6.84 (m, 1H); 6.68 (m, 1H); 3.76 (m, 4H); 3.52 (m, 4H); 2.96 (m, 2H); 2.37 (M, 2H); 1.64 (m, 2H); 1.37 (s, 9H)

### Preparation of tert-butyl 4-(4-(pyrimidin-2-yl)piperazin-1-yl) (4,4-²H₂)butylcarbamate

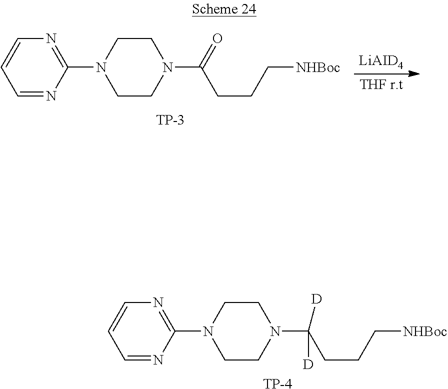

A solution of tert-butyl 4-oxo-4-(4-(pyrimidin-2-yl)piperazin-1-yl) butylcarbamate (6.43 g, 18.4 mmol) in THF (100 ml) was treated with LiAlD₄ (0.85 mg, 20.3 mmol) at 0° C. The resulting mixture was stirred at room temperature for overnight. The reaction mixture was treated with Na₂SO₄.10H₂O for 1.5 hrs. Filtered to remove the solid, the filtrate was concentrated in vacuo to give tert-butyl 4,4-di-deuterated-4-(4-(pyrimidin-2-yl)piperazin-1-yl) (4,4-²H₂)butylcarbamate (5.41 g, 87.2% yield) as a colorless oil, which was used in next step without further purification.

LCMS: RT=0.894 min m/z=338.2 (M+1)

### Preparation of tert-butyl 4-(4-(5-bromopyrimidin-2-yl)piperazin-1-yl) (4,4-²H₂)butyl carbamate

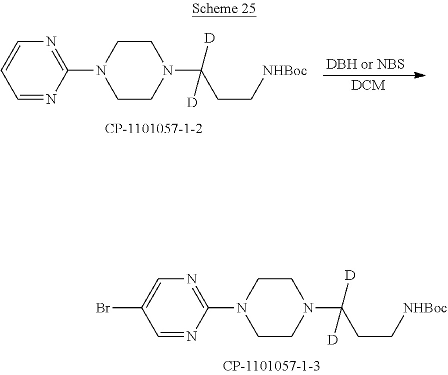

A solution of tert-butyl 4-(4-(pyrimidin-2-yl)piperazin-1-yl) (4,4-²H₂)butylcarbamate (8.204 g, 24.34 mmol) and NBS (5.2 g, 29.21 mmol) in DCM (250 ml) was stirred at room temperature for overnight. The reaction mixture was diluted with DCM (200 ml), washed with a saturated solution of NaHCO₃ twice, and then with brine, dried over MgSO₄, concentrated in vacuo to give tert-butyl 4-(4-(5-bromopyrimidin-2-yl)piperazin-1-yl) (4,4-²H₂)butylcarbamate (4.1 g, 40.4% yield). The crude product was used in next step without further purification.

LCMS: RT=1.071 min m/z=416.2 (M+1).

### Preparation of 4-(4-(5-bromopyrimidin-2-yl)piperazin-1-yl) (4,4-²H₂)butan-1-amine

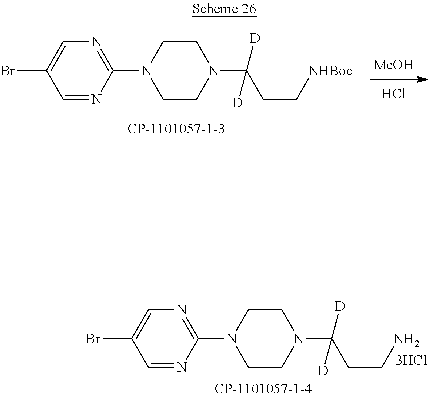 [SMILES: "MeOH" ; [2H]C([2H])(CCNC(=O)OC(C)(C)C)N1CCN(c2ncc(Br)cn2)CC1 ; Cl ; [2H]C([2H])(CCN)N1CCN(c2ncc(Br)cn2)CC1]

A solution of tert-butyl 4-(4-(5-bromopyrimidin-2-yl)piperazin-1-yl) (4,4-²H₂)butyl carbamate (4.1 g, 9.9 mmol) in methanol (50 ml) was treated with 3.0 M HCl/MeOH (50 ml, 250 mmol) for 3 hrs. And then the reaction mixture was concentrated in vacuo to give 4-(4-(5-bromopyrimidin-2-yl)piperazin-1-yl) (4,4-²H₂)butan-1-amine hydrochloride (3.41 g, ˜100% yield).

LCMS: RT=0.925 min m/z=318.0 (M+1).

### Preparation of N-[4-[4-(5-bromopyrimidin-2-yl)-1-piperazinyl]-(4,4-²H₂)butyl]-2,3-endo-bicyclo[2.2.1]heptanedicarboximide

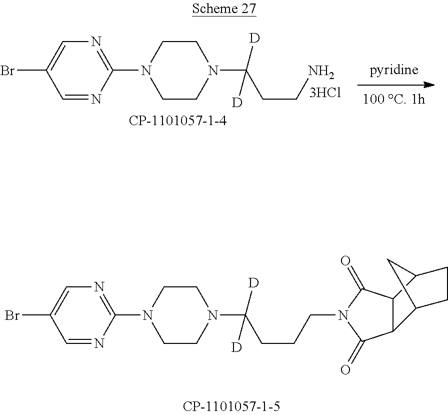 [SMILES: [2H]C([2H])(CCN)N1CCN(c2ncc(Br)cn2)CC1 ; "3HCl" ; "pyridine" ; [2H]C([2H])(CCCN1C(=O)C2C3CCC(C3)C2C1=O)N1CCN(c2ncc(Br)cn2)CC1]

A solution of bicyclo[2.2.1]heptane-2,3-dicarboxylic anhydride (0.67 g, 4.01 mmol) and 4-(4-(5-bromopyrimidin-2-yl)piperazin-1-yl) (4,4-²H₂)butan-1-amine hydrochloride (2.043 g, 4.81 mmol) in pyridine (15 ml) was heated at reflux for an hour. The reaction mixture was concentrated in vacuo to remove the solvent. The residue was diluted with ethyl acetate (300 ml) and then washed with water (100 ml×2), dried over Na₂SO₄, concentrated in vacuo to give N-[4-[4-(5-bromopyrimidin-2-yl)-1-piperazinyl]-(4,4-²H₂)butyl]-2,3-endo-bicyclo[2.2.1]heptanedicarboximide (1.686 g, 75% yield) as colorless oil.

LCMS: RT=1.849 min, m/z=464.1 (M+1)

### Preparation of N-[4-[4-(5-deuteratedpyrimidin-2-yl)-1-piperazinyl]-(4,4-²H₂)butyl]-2,3-endo-bicyclo[2.2.1]heptanedicarboximide

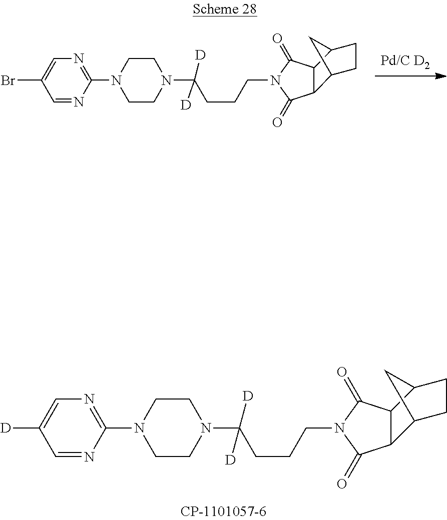 [SMILES: [2H]C([2H])(CCCN1C(=O)C2C3CCC(C3)C2C1=O)N1CCN(c2ncc(Br)cn2)CC1 ; [2H]c1cnc(N2CCN(C([2H])([2H])CCCN3C(=O)C4C5CCC(C5)C4C3=O)CC2)nc1]

A solution of N-[4-[4-(5-bromopyrimidin-2-yl)-piperazin-1-yl]-(4,4-²H₂)butyl]-2,3-endo-bicyclo[2.2.1]heptanedicarboximide (1.7 g, 3.7 mmol) in EtOD (30 ml) was treated with Pd/C (170 mg, 10% Pd on C, treated with D₂O before used) at 50° C. for 2 hours. The reaction mixture was cooled to room temperature and filtered to remove the solid. The filtrate was purified by prep-HPLC directly (A: water contained 10 mMol NH₄HCO₃ B: CH₃CN) to give N-[4-[4-(5-(²H)pyrimidin-2-yl)-1-piperazinyl]-(4,4-²H₂)butyl]-2,3-endo-bicyclo[2.2.1]heptane dicarboximide (120 mg, 8.4%)

LCMS: RT=1.666 min, m/z=387.2 (M+1)

HPLC: RT=4.118 min, 100% (254 nm), 97.6% (214 nm)

¹HNMR (400 MHz, CDCl3): δ 8.31 (s, 2H); 3.82 (m, 4H); 3.51 (m, 2H); 3.1 (s, 2H); 2.76 (m, 2H); 2.47 (d, 4H); 1.5˜1.62 (m, 10H)

### Preparation of N-[4-[4-(5-(²H)pyrimidin-2-yl)-1-piperazinyl]-(4,4-²H₂)butyl]-2,3-endo-bicyclo[2.2.1]heptanedicarboximide citric salt

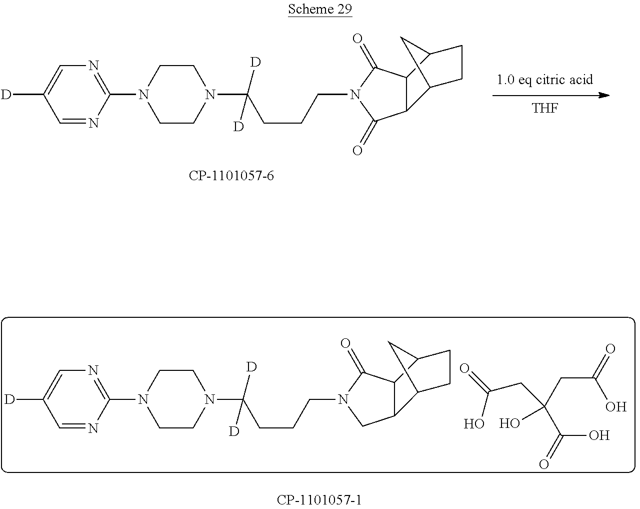

A solution of N-[4-[4-(5-(²H)pyrimidin-2-yl)-piperazin-1-yl]-(4,4-²H₂)butyl]-2,3-endo-bicyclo[2.2.1]heptane dicarboximide (280 mg, 0.725 mol) and citric acid (152.4 mg, 0.725 mol) in THF (50 ml) was stirred at room temperature for 2 hours, and then the reaction solution was let to stay for 2 days. The solid was separated by filtration. The collected solid was re-dissolved in water and then dried by lyophilization.

Mp: 60.5˜68.1° C.,

LCMS: RT=1.041 min, m/z=387.1 (M+1)

¹HNMR (400 MHz, CD3OD): δ 8.41 (s, 2H); 4.11 (m, 4H); 3.53 (t, 2H); 3.26 (m, 4H); 3.18 (m, 2H); 2.84˜2.89 (d, 2H); 2.74˜2.78 (d, 2H); 2.71 (m, 2h); 1.59˜1.81 (m, 8H); 1.20 (m, 2H).

### Example 13

### Synthesis of (1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione

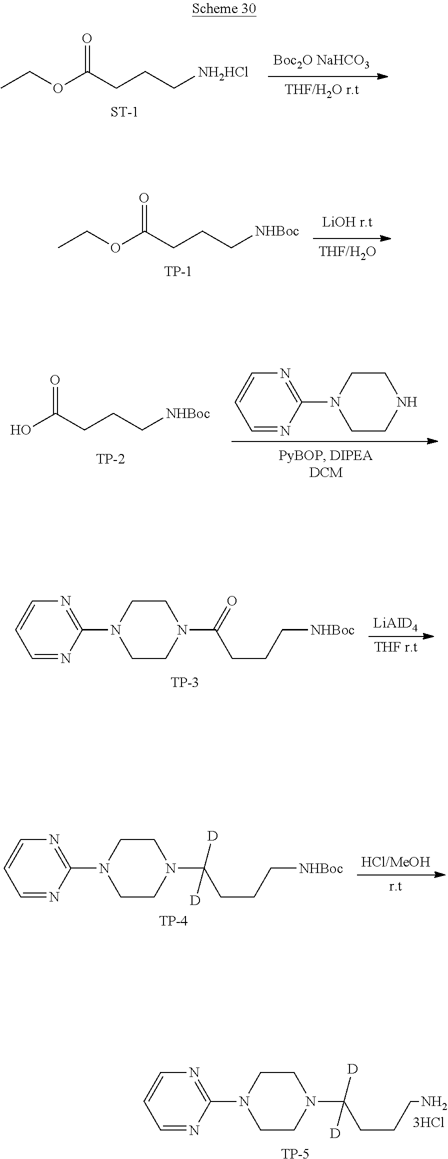

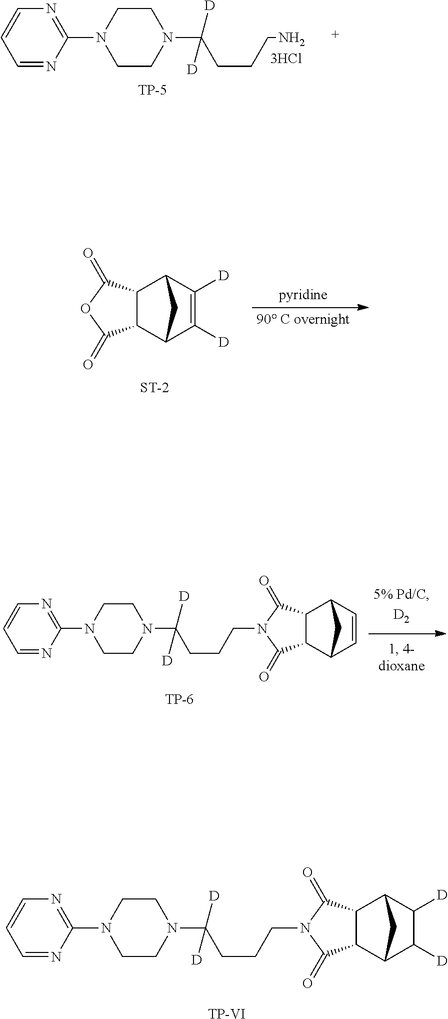

### Preparation of tert-butyl 4-oxo-4-(4-(pyrimidin-2-yl)piperazin-1-yl) butylcarbamate

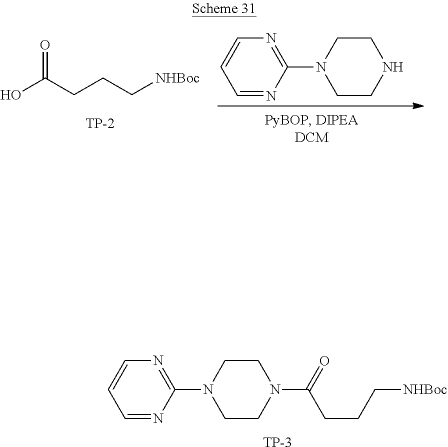

A solution of 4-(tert-butoxycarbonylamino)butanoic acid (4.54 g, 22.4 mmol), PyBOP (14.3 g, 27.4 mmol) and DIPEA (7.1 g, 54.9 mmol) in DCM (100 ml) was stirred at room temperature for 10 min, and then 2-(piperazin-1-yl)pyrimidine (3.0 g, 18.6 mmol) was added. The resulting mixture was stirred at room temperature overnight. The reaction mixture was diluted with DCM (100 ml), washed with water (150 ml×2). The organic phase was dried over Na₂SO₄, concentrated in vacuo to give the crude product which was purified by combiflash column (100% EtOAc) to afford tert-butyl 4-oxo-4-(4-(pyrimidin-2-yl)piperazin-1-yl)butylcarbamate (3.57 g, 55.1% yield) as a white oil.

LCMS: RT=1.23 min, m/z=350.1 (M+1).

¹HNMR (400 MHz, DMSO-d6): δ 8.39 (d, 2H), 6.84 (m, 1H), 6.68 (m, 1H), 3.76 (m, 4H), 3.52 (m, 4H), 2.96 (m, 2H), 2.37 (m, 2H), 1.64 (m, 2H), 1.37 (s, 9H)

### Preparation of tert-butyl 4-(4-(pyrimidin-2-yl)piperazin-1-yl)-(4,4-²H₂)butylcarbamate

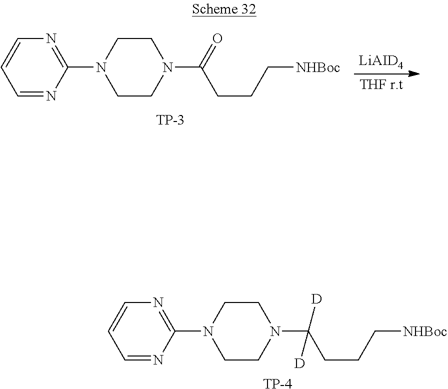

A solution of tert-butyl 4-oxo-4-(4-(pyrimidin-2-yl)piperazin-1-yl)butylcarbamate (0.5 g, 1.433 mmol) in THF (80 ml) was treated with LiAlD₄ (60.2 mg, 1.433 mmol) at 0° C. The resulting mixture was stirred at room temperature for overnight. The reaction mixture was treated with Na₂SO₄.10H₂O for 1.5 hrs. Reaction mixture was filtered to remove the solid, the filtrate was concentrated in vacuo to give tert-butyl 4-(4-(pyrimidin-2-yl)piperazin-1-yl)-(4,4-²H₂)— butylcarbamate (420 mg, 87% yield) as a colorless oil, which was used in next step without further purification.

LCMS: RT=0.894 min, m/z=338.2 (M+1).

### Preparation of 4-(4-(pyrimidin-2-yl)piperazin-1-yl)-(4,4-²H₂)butan-1-amine

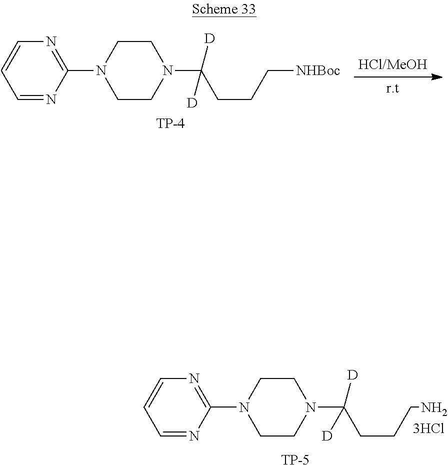

A solution of tert-butyl 4-(4-(pyrimidin-2-yl)piperazin-1-yl)-(4,4-²H₂)— butylcarbamate (420 mg, 1.246 mmol) in methanol (50 ml) was treated with 3.0 M HCl/MeOH (50 ml, 150 mmol) for 3 hrs. Then the reaction mixture was concentrated in vacuo to give 4-(4-(pyrimidin-2-yl)piperazin-1-yl)-(4,4-²H₂)— butan-1-amine (310 mg, 80.8% yield)

LCMS: RT=0.309 min, m/z=238.2 (M+1).

¹HNMR (400 MHz, CD3OD): δ 8.73 (d, 2H), 7.17 (dd, 1H), 4.87 (m, 1H), 4.27 (m, 1H), 3.86 (m, 3H), 3.49 (m, 2H), 3.31 (m, 1H), 3.08 (m, 2H), 1.97 (m, 2H), 1.81 (m, 2H)

### Preparation of N-[4-[4-(pyrimidin-2-yl)piperazin-1-yl]-(4,4-²H₂)-butyl]-2,3-endo-bicyclo[2.2.1]hept-5-ene-dicarboximide

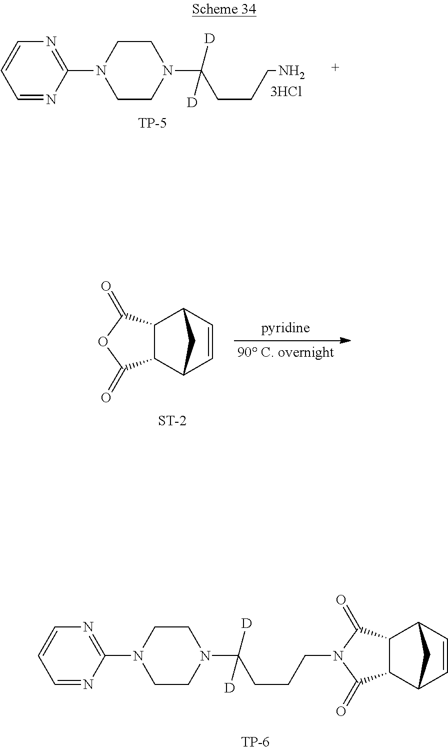

A solution of 4-(4-(pyrimidin-2-yl)piperazin-1-yl)-(4,4-²H₂)butan-1-amine (200 mg, 1.22 mmol) and bicyclo[2.2.1]hept-5-ene-2,3-dicarboxylic acid anhydride (420 mg, 1.22 mmol) in pyridine (5 ml) was heated to 100° C. for 3 hrs. The reaction mixture was cooled to room temperature, and then diluted with EtOAc (150 ml), washed with water. The organic phase was dried over Na₂SO₄, and concentrated in vacuo to give N-[4-[4-(pyrimidin-2-yl)piperazin-1-yl]-(4,4-²H₂)butyl]-2,3-endo-bicyclo[2.2.1]hept-5-ene-dicarboximide (284 mg, 60.8% yield) as a colorless oil.

LCMS: RT=0.872 min, m/z=384.1 (M+1).

### (1R,2R,6S,7S)-4-{4-[4-(pyrimidin-2-yl)piperazin-1-yl]-(4,4-²H₂)butyl}-(8,9-²H₂)-4-azatricyclo[5.2.1.0^{2,6}]decane-3,5-dione

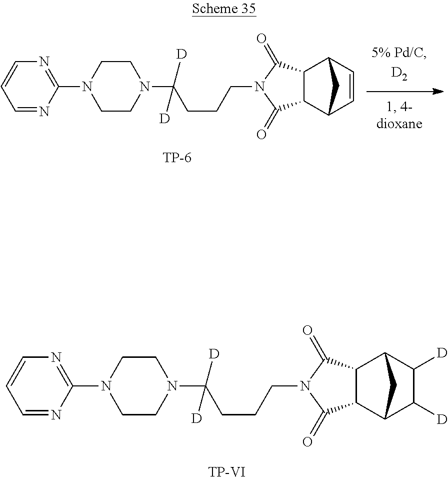

A solution of N-[4-[4-(pyrimidin-2-yl)piperazin-1-yl]-(4,4-²H₂)butyl]-2,3-endo-bicyclo[2.2.1]hept-5-ene-dicarboximide (284 mg, 0.742 mmol) in 1,4-dioxane (100 ml) was treated with Pd/C (30 mg, 5%) under 1 atm of D₂ at room temperature overnight. The reaction mixture was filtered to remove the catalyst, and the filtrate was concentrated in vacuo to give the crude product, which was purified by prep-HPLC to afford (1R,2R,6S,7S)-4-{4-[4-(pyrimidin-2-yl)piperazin-1-yl]-(4,4-²H₂)butyl}-(8,9-²H₂)-4-azatricyclo[5.2.1.0^{2,6}]decane-3,5-dione (21 mg, 73.1% yield) as a colorless oil.

LCMS: RT=0.889 min, m/z=388.1 (M+1)

HPLC: RT=4.739 min, 100% (214 nm, 254 nm)

¹HNMR (400 MHz, CD₃OD): δ 8.30 (dd, 2H), 6.59 (t, 1H), 5.76 (m, 1H), 4.80 (m, 2H), 3.39˜3.60 (m, 6H), 3.02 (m, 2H), 2.68˜2.72 (m, 4H), 1.72 (m, 2H), 1.49˜1.63 (m, 4H), 1.10 (m, 2H).

### Example 14

### Synthesis of (1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione

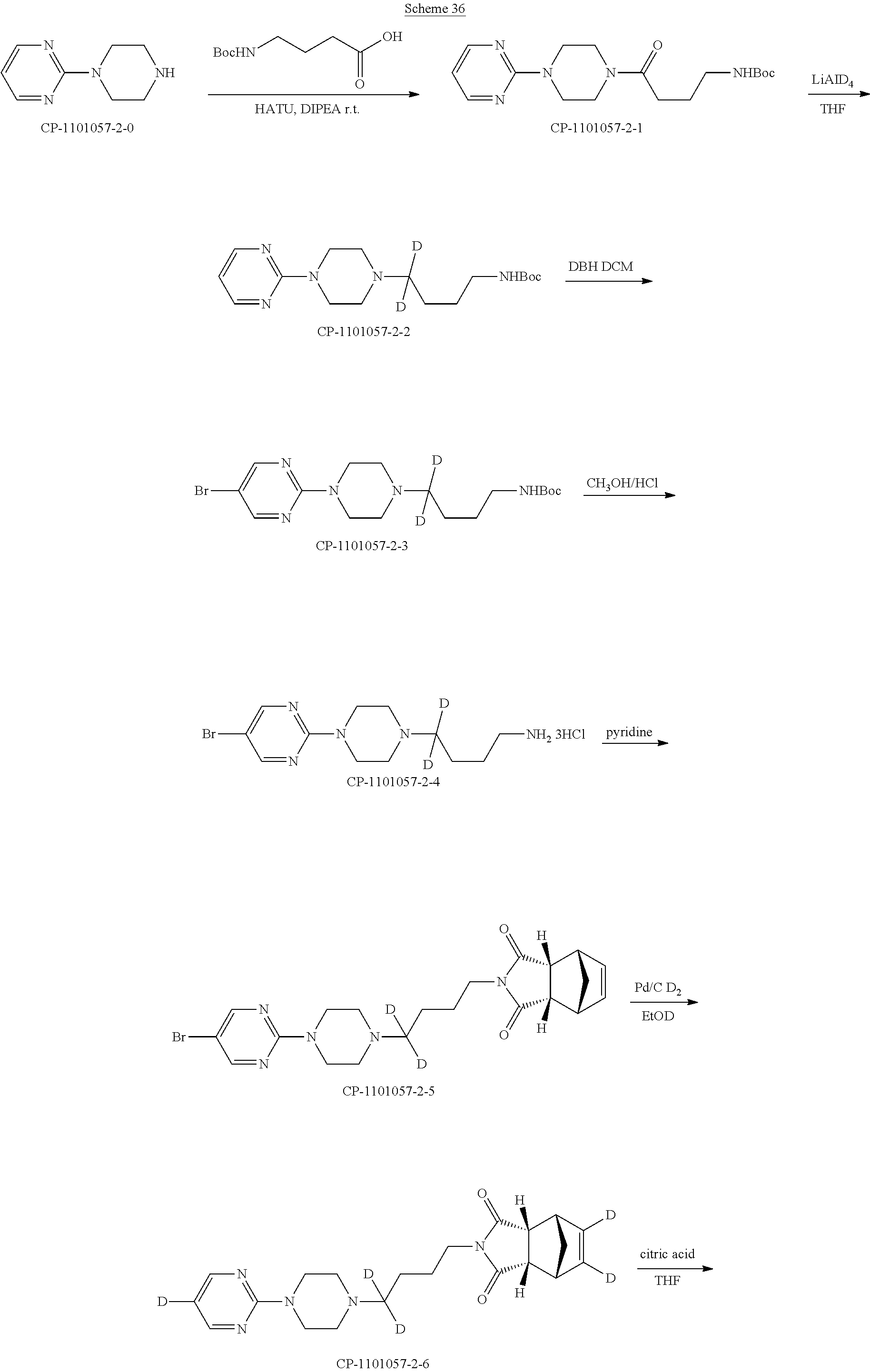

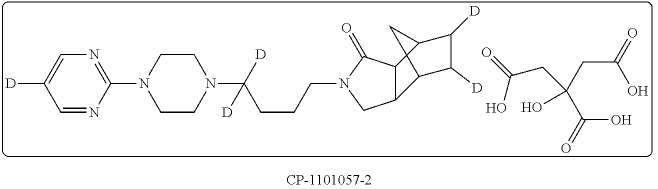

### Preparation of ethyl-4-(tert-butoxycarbonylamino)butanoate

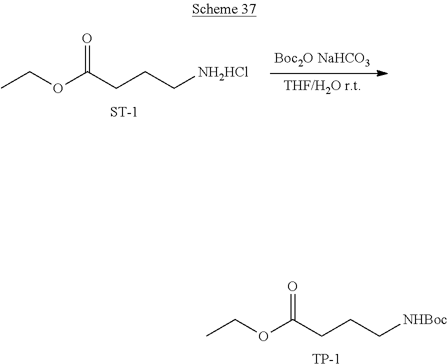

A solution of ethyl 4-aminobutanoate hydrochloride (3.0 g, 18 mmol), Boc₂O (3.91 g, 18 mmol) and K₂CO₃ (7.5 g, 54 mmol) in THF/H₂O (50 ml, 1:1) was stirred at room temperature for 4 hrs. The reaction mixture was diluted with water (100 ml), extracted with ethyl acetate (100 ml×2). The combined organic phase was washed with brine, dried over Na₂SO₄, and concentrated in vacuo to give ethyl 4-(tert-butoxycarbonylamino) butanoate (3.6 g, 86.6% yield) as a white oil.

LC-MS: RT=1.304 min, m/z=118 (M−100)

### Preparation of 4-(tert-butoxycarbonylamino)butanoic acid

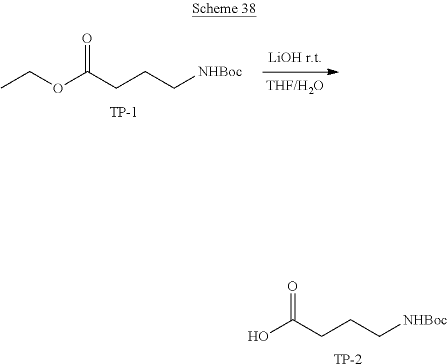

A suspension of ethyl 4-(tert-butoxycarbonylamino)butanoate (3.6 g, 15.58 mmol) and LiO.HH₂O (1.96 g, 46.74 mmol) in THF/H₂O (60 ml, 1:1) was stirred at room temperature for 4 hrs. The reaction mixture was diluted with water (150 ml), extracted with Et₂O (100 ml×2). The aqueous was acidified with 2.0 N HCl to pH 3-4, and then extracted with ethyl acetate (150 ml×2). The combined organic phase was dried over Na₂SO₄, and concentrated in vacuo to afford 4-(tert-butoxycarbonylamino)butanoic acid (4.5 g, 100%) as a white oil.

LC-MS: RT=1.376 min, m/z=226 (M+Na)

¹H-NMR (400 MHz, CDCl3): δ12.05 (s, 1H), 6.85 (br, 1H), 2.94 (m, 2H), 2.21 (m, 2H), 1.60 (m, 2H), 1.38 (s, 9H).

### Preparation of tert-butyl 4-oxo-4-(4-(pyrimidin-2-yl)piperazin-1-yl) butylcarbamate

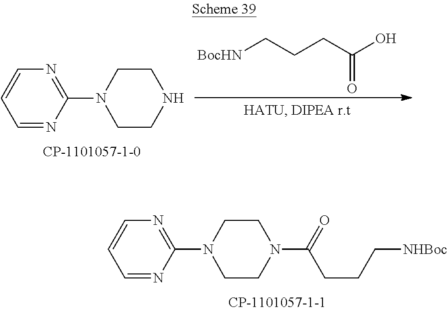 [SMILES: c1cnc(N2CCNCC2)nc1 ; CC(C)(C)OC(=O)NCCCC(=O)O ; CC(C)(C)OC(=O)NCCCC(=O)N1CCN(c2ncccn2)CC1]

A solution of 4-(tert-butoxycarbonylamino)butanoic acid (10.4 g, 21.5 mmol), HATU (10.4 g, 27.4 mmol) and DIPEA (9.54 ml, 54.9 mmol) in DCM (100 ml) was stirred at room temperature for 10 min and then 2-(piperazin-1-yl)pyrimidine (3.0 g, 18.6 mmol) was added. The resulting mixture was stirred at room temperature for overnight. The reaction mixture was diluted with DCM (100 ml), washed with water (150 ml×2). The organic phase was dried over Na₂SO₄, concentrated in vacuo to give the crude product which was purified by combiflash column (100% EtOAc) to afford tert-butyl 4-oxo-4-(4-(pyrimidin-2-yl)piperazin-1-yl) butylcarbamate (6.43 g, ˜100% yield) as a white oil.

LC-MS: RT=1.23 min, m/z=350.1 (M+1).

¹H-NMR (400 MHz, DMSO-d6): δ: 8.39 (d, 2H); 6.84 (m, 1H); 6.68 (m, 1H); 3.76 (m, 4H); 3.52 (m, 4H); 2.96 (m, 2H); 2.37 (M, 2H); 1.64 (m, 2H); 1.37 (s, 9H)

### Preparation of tert-butyl 4-(4-(pyrimidin-2-yl)piperazin-1-yl) (4-²H₂)butylcarbamate

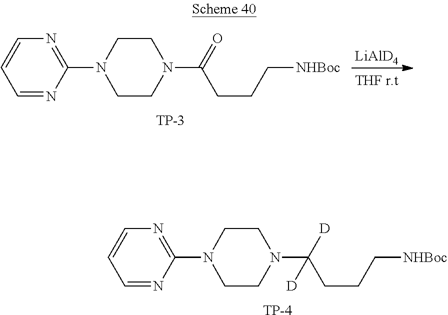

A solution of tert-butyl 4-oxo-4-(4-(pyrimidin-2-yl)piperazin-1-yl) butylcarbamate (6.43 g, 18.4 mmol) in THF (100 ml) was treated with LiAlD₄ (0.85 mg, 20.3 mmol) at 0° C., and then the resulting mixture was stirred at room temperature for overnight. The reaction mixture was treated with Na₂SO₄.10H₂O for 1.5 hrs. And then filter to remove the solid, the filtrate was concentrated in vacuo to give tert-butyl 4-(4-(pyrimidin-2-yl)piperazin-1-yl) (4-²H₂)butylcarbamate (5.41 g, 87.2% yield) as a colorless oil, which was used in next step without further purification.

The end-product was analyzed to determine LCMS retention time and mass:

LC-MS: RT=0.894 min m/z=338.2 (M+1)

### Preparation of tert-butyl 4-(4-(5-bromopyrimidin-2-yl)piperazin-1-yl) (4-²H₂)— butylcarbamate

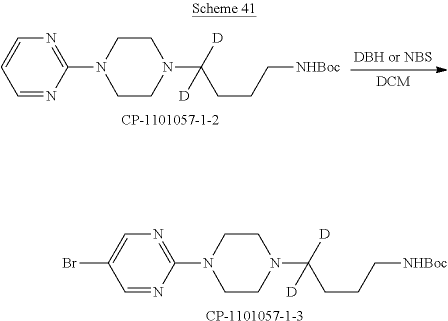

A solution of tert-butyl 4-(4-(pyrimidin-2-yl)piperazin-1-yl) (4-²H₂)butyl carbamate (8.204 g, 24.34 mmol) and NBS (5.2 g, 29.21 mmol) in DCM (250 ml) was stirred at room temperature for overnight. The reaction mixture was diluted with DCM (200 ml), washed with a saturated solution of NaHCO₃ twice, and then with brine, dried over MgSO₄, concentrated in vacuo to give tert-butyl 4-(4-(5-bromopyrimidin-2-yl)piperazin-1-yl) (4-²H₂)butylcarbamate (4.1 g, 40.4% yield). The crude product was used in next step without further purification.

The end-product was analyzed to determine LCMS retention time and mass:

LCMS: RT=1.071 min m/z=416.2 (M+1).

### Preparation of 4-(4-(5-bromopyrimidin-2-yl)piperazin-1-yl) (4-²H₂)butan-1-amine

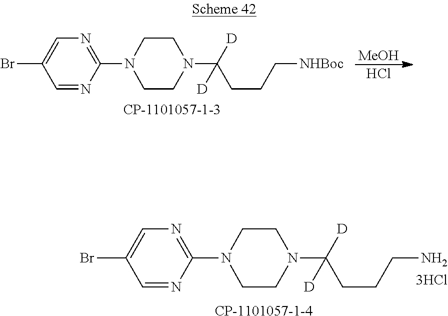

A solution of tert-butyl 4-(4-(5-bromopyrimidin-2-yl)piperazin-1-yl) (4-²H₂)— butylcarbamate (4.1 g, 9.9 mmol) in methanol (50 ml) was treated with 3.0 M HCl/MeOH (50 ml, 250 mmol) for 3 hrs. And then the reaction mixture was concentrated in vacuo to give 4-(4-(5-bromopyrimidin-2-yl)piperazin-1-yl) (4-²H₂)butan-1-amine hydrochloride (3.41 g, ˜100% yield).

LC-MS: RT=0.925 min m/z=318.0 (M+1).

### Preparation of N-[4-[4-(5-bromopyrimidin-2-yl)piperazin-1-yl]-(4-²H₂)butyl]-2,3-endo-bicyclo[2.2.1]hept-5-ene-dicarboximide

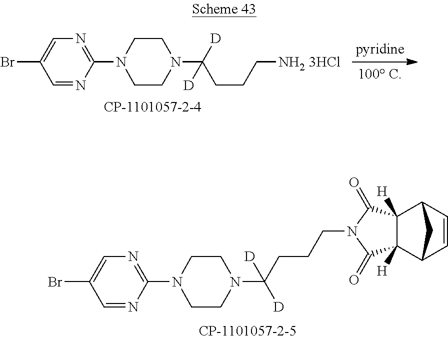

A solution of bicyclo[2.2.1]heptane-5-ene-2,3-dicarboxylic anhydride (0.335 g, 2.05 mmol) and 4-(4-(5-bromopyrimidin-2-yl)piperazin-1-yl) (4-²H₂)butan-1-amine hydrochloride (0.87 g, 2.05 mmol) in pyridine (6 ml) was heated at reflux for an hour. The reaction mixture was concentrated in vacuo to remove the solvent. The residue was diluted with ethyl acetate (100 ml) and then washed with water (50 ml×2), dried over Na₂SO₄, concentrated in vacuo to give N-[4-[4-(5-bromopyrimidin-2-yl)piperazin-1-yl]-(4-²H₂)butyl]-2,3-endo-bicyclo[2.2.1]heptane-5-ene-dicarboximide (0.74 g, 78.1% yield) as colorless oil.

The end-product was analyzed to determine the LCMS retention time and mass:

LC-MS: RT=1.041 min, m/z=462.0 (M+1)

### Preparation of ((1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione)

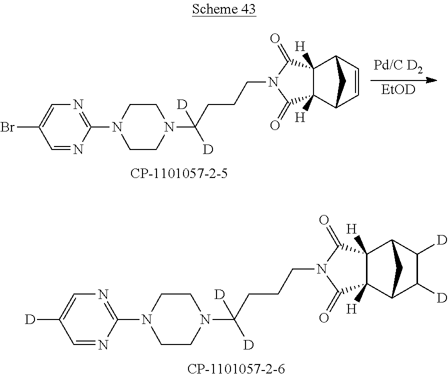 [SMILES: "EtOD" ; [2H]C([2H])(CCCN1C(=O)[C@@H]2[C@H](C1=O)[C@@H]1C=C[C@H]2C1)N1CCN(c2ncc(Br)cn2)CC1 ; [2H]c1cnc(N2CCN(C([2H])([2H])CCCN3C(=O)[C@@H]4[C@H](C3=O)[C@H]3C[C@@H]4C([2H])C3[2H])CC2)nc1]

A solution of N-[4-[4-(5-bromopyrimidin-2-yl)piperazin-1-yl]-(4-²H₂)butyl]-2,3-endo-bicyclo[2.2.1]heptane-5-ene-dicarboximide (0.74 g, 1.61 mmol) in EtOD (6 ml) was treated with Pd/C (74 mg, 5% Pd on C treated with D₂O before used) under 1 atm. of D₂ at 40° C. for 2 hours. The reaction mixture was filtered to remove the catalyst, and the filtrate was concentrated in vacuo to give the crude product, which was purified by prep-HPLC to afford (1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione (43 mg, 6.9% yield) as a colorless oil.

The end-product was analyzed to determine the melting point (Mp), LCMS retention time and mass, and the ¹H-NMR chemical shifts:

LC-MS: RT=1.665 min, m/z=389.2 (M+1)

HPLC: RT=4.088 min 100% (254 nm), 97.6% (214 nm)

¹H-NMR (400 MHz, CCl₃D): δ 8.31 (s, 2H); 3.82 (m, 4H); 3.50 (m, 2H); 3.063 (s, 2H); 2.75 (s, 2H); 2.49 (m, 4H); 1.61 (m, 8H)

### Preparation of ((1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione) citrate

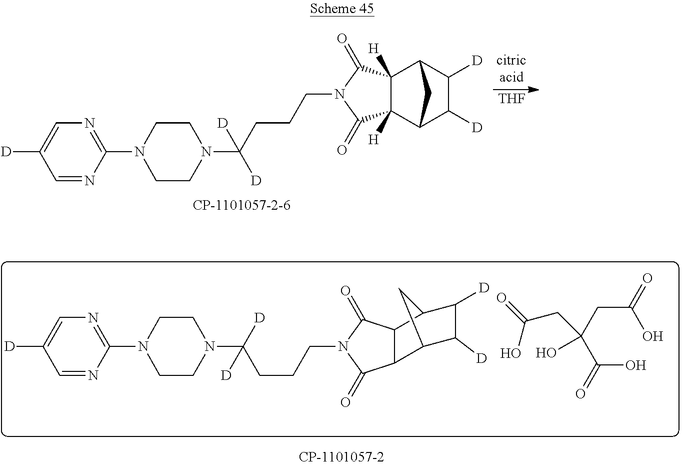

A solution of ((1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione) (190 mg, 0.49 mmol) and citric acid (102.9 mg, 0.49 mmol) in THF (50 ml) was stirred at room temperature for 2 hours, and then the reaction solution was let to stay for 2 days. The solid was separated by filtration. The collected solid was re-dissolved in water and then lyophilized.

The end-product was analyzed to determine the melting point (Mp), LCMS retention time and mass, and the ¹H-NMR chemical shifts:

Mp: 46.4° C.˜57.3° C.

LC-MS: RT=1.018 min, m/z=389.2 (M+1)

¹H-NMR (400 MHz, CD₃OD): δ 8.43 (s, 2H); 4.15 (br, 4H); 3.53 (t, 2H); 3.37 (m, 4H); 3.19 (m, 2H); 2.89˜2.93 (d, 2H); 2.77˜2.82 (d, 2H); 2.70 (m, 2H); 1.63˜1.89 (m, 6H); 1.18 (m, 2H)

### Example 15

### Synthesis of (1R,2R,6S,7S)-4-{4-[4-((5-²H)pyrimidin-2-yl)piperazin-1-yl]-(4,4-²H₂)butyl}-(2,6-²H₂)-4-azatricyclo[5.2.1.0^{2,6}]decane-3,5-dione

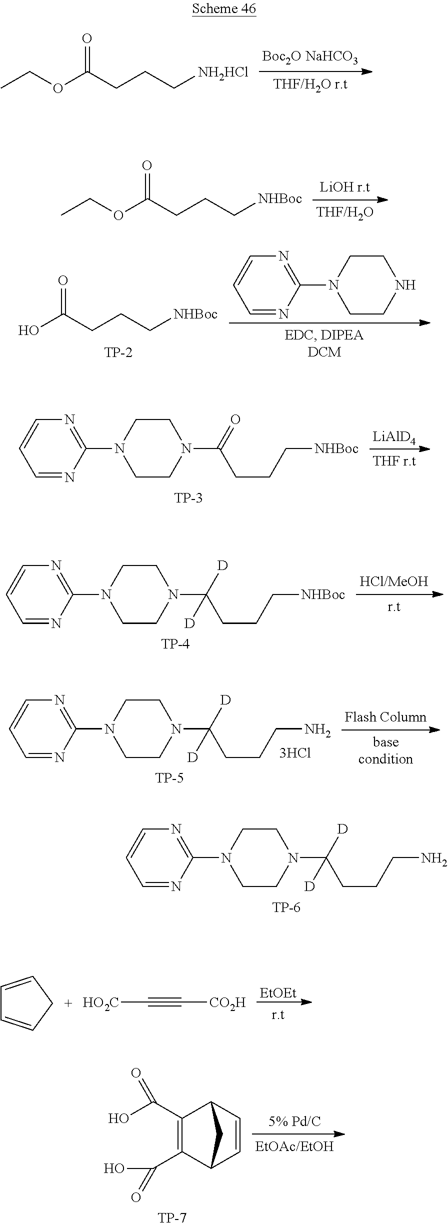

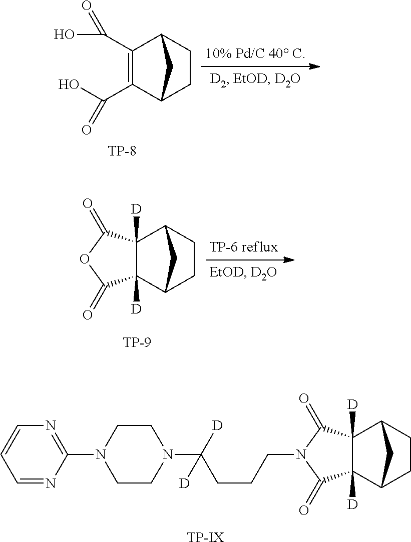

### Preparation of bicyclo[2.2.1]hepta-2,5-diene-2,3-dicarboxylic acid

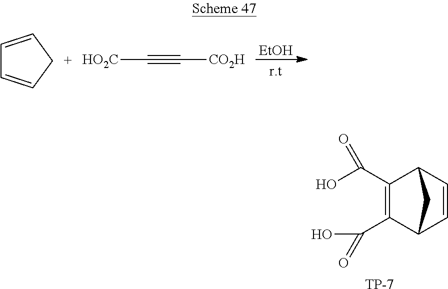

To a solution of but-2-ynedioic acid (5.0 g, 43.8 mmol) in ether (40 ml) was added dropwise of freshly distilled cyclopenta-1,3-diene (3.8 ml, 45.6 mmol) at room temperature under N₂ atmosphere. The reaction mixture, which warmed slightly after a short period of time, was stirred at room temperature. Evaporation of the solvent in vacuo and trituration of the residue in petroleum ether, which was then crystallized in water to give bicyclo[2.2.1]hepta-2,5-diene-2,3-dicarboxylic acid (2.6 g, 40% yield) as a white solid.

LCMS: RT=0.78 min, m/z=181.0 (M+1).

### Preparation of bicyclo[2.2.1]hept-2-ene-2,3-dicarboxylic acid

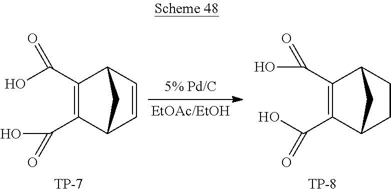

A solution of bicyclo[2.2.1]hepta-2,5-diene-2,3-dicarboxylic acid (2.5 g, 139 mmol) in EtOAc (36 ml) and EtOH (6 ml) was treated with Pd/C (250 mg) at room temperature under H₂ atmosphere for 3 hrs. The reaction mixture was filtered to remove the solid. The filtrate was concentrated in vacuo to give bicyclo[2.2.1]hept-2-ene-2,3-dicarboxylic acid (2.6 g, 100% yield) as a white solid.

LCMS: RT=0.863, min m/z=183.0 (M+1).

### Preparation of 2,3-²H₂-bicyclo[2.2.1]heptane-2,3-dicarboxylic acid

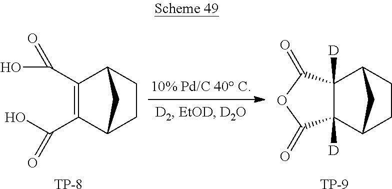

A solution of bicyclo[2.2.1]hept-2-ene-2,3-dicarboxylic acid (400 mg, 2.2 mmol) in EtOD (15 ml) and a few drops of D₂O was treated with Pd/C (40 mg, 10% treated with D₂O before used) under 1 atm of D₂ at 45° C. for 3 hrs. After removal of the solid, the reaction solution was used in next step directly.

LCMS: RT=1.259 min, m/z=169.0 (M+1)

### Preparation of (1R,2R,6S,7S)-4-{4-[4-((5-²H)pyrimidin-2-yl)piperazin-1-yl]-(4,4-²H₂)butyl}-(2,6-²H₂)-4-azatricyclo[5.2.1.0^{2,6}]decane-3,5-dione

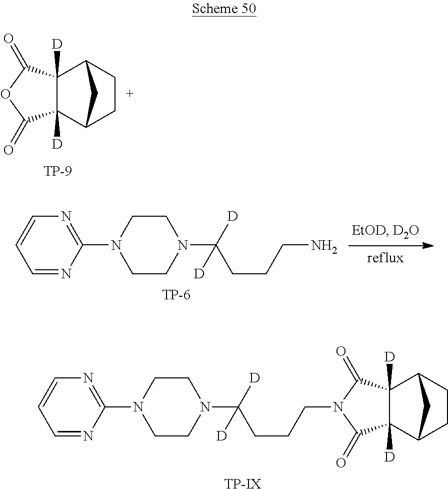

A solution of 2,3-²H₂-bicyclo[2.2.1]heptane-2,3-dicarboxylic acid (2.2 mmol) and 4,4-²H₂-4-(4-(pyrimidin-2-yl)piperazin-1-yl)butan-1-amine (564.3 mg, 2.38 mmol) in EtOD (50 ml) was heated at reflux overnight. The reaction mixture was concentrated in vacuo to remove the solvent. The crude product was purified by prep-HPLC to give the title compound (140 mg, 16.4% yield) as colourless oil.

The end-product was analyzed to determine the melting point (Mp), LCMS retention time and mass, and the ¹H-NMR chemical shifts:

LCMS: RT=0.886 min, m/z=388.2 (M+1)

HPLC: RT=5.073 min, 100% (214 nm, 254 nm)

¹HNMR (400 MHz, CDCl₃): δ 8.36 (d, 2H), 6.65 (t, 1H), 4.85 (m, 3H), 3.46˜3.67 (m, 5H), 2.74 (m, 3H), 2.63 (m, 1H), 1.56˜1.81 (m, 7H), 1.16˜1.26 (m, 3H).

### Example 16

### (1R,2R,6S,7S)-4-{4-[4-(pyrimidin-2-yl)piperazin-1-yl]-(2,2,3,3,4,4-²H₆)butyl}-4-azatricyclo[5.2.1.0^{2,6}]decane-3,5-dione

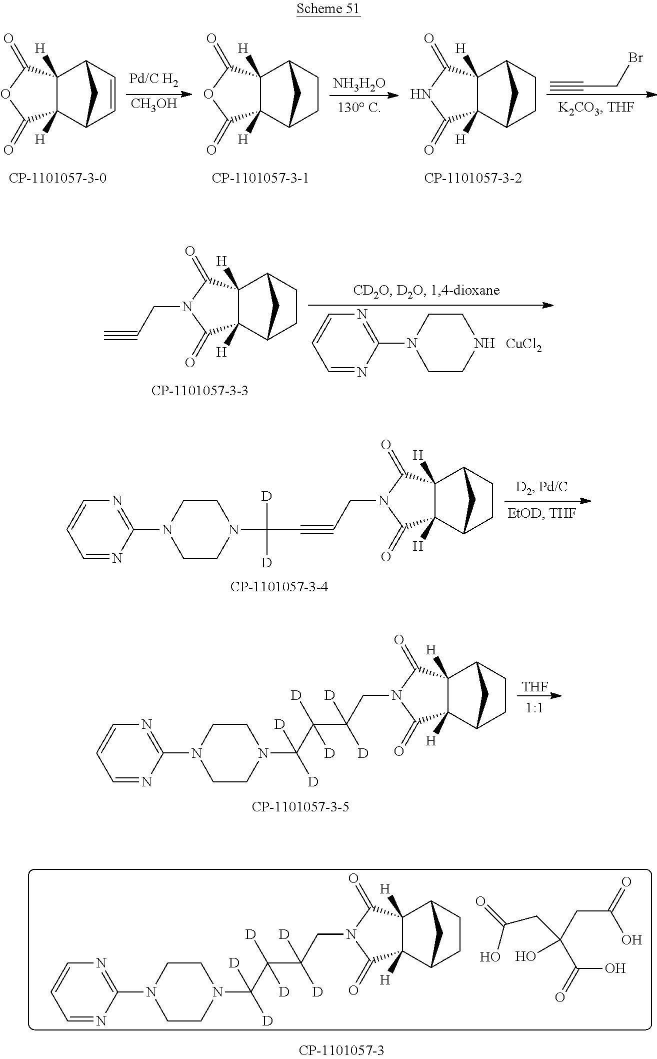

### Preparation of bicyclo[2.2.1]heptane-2,3-dicarboxylic acid anhydride

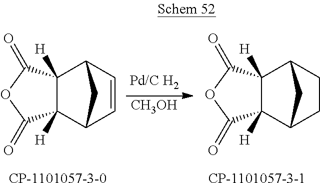

A solution of bicyclo[2.2.1]hept-5-ene-2,3-dicarboxylic acid anhydride (6.0 g, 36.6 mmol) in methanol (150 ml) was treated with Pd/C (0.6 g, 10% Pd on C) at room temperature under 1 atm of H₂ atmosphere for overnight. The reaction mixture was filtered to remove the solid; the filtrate was concentrated in vacuo to give the target (6.1 g, ˜100% yield) as a white solid.

LC-MS: RT=1.488 min, m/z=167 (M+1)

### Preparation of bicyclo[2.2.1]heptane-2,3-dicarboximide

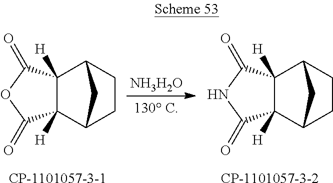

To a stirred solution of bicyclo[2.2.1]heptane-2,3-dicarboxylic acid anhydride (2.0 g, 12.05 mmol) in THF (15 ml) was added 30% aqueous ammonia (5 ml) at room temperature. The mixture was slowly heated to 130° C., kept for 2 hours at the same temperature and cooled. The resulting precipitated was collected by filtration and washed with n-hexane to give bicyclo[2.2.1]heptane-2,3-dicarboximide (1.502 g, 75.54% yield).

LC-MS: RT=0.992 min, m/z=166.1 (M+1)

### Preparation of N-propargylbicyclo[2.2.1]heptane-2,3-dicarboximide

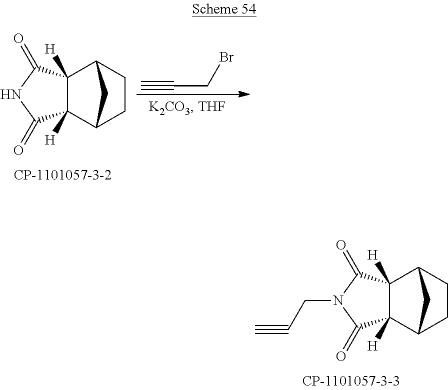

A mixture of bicyclo[2.2.1]heptane-2,3-dicarboximide (1.504 g, 9.12 mmol), propargyl bromide (1.19 g, 10.03 mmol) and anhydrous K₂CO₃ (1.9 g, 13.7 mmol) in CH₃CN was refluxed with stirring under N₂ for 3 hours, cooled and filtered. The filtrated was diluted with ethyl acetate (200 ml), washed with brine twice, the organic phase was dried over Na₂SO₄, concentrated in vacuo to give N-propargylbicyclo[2.2.1]heptane-2,3-dicarboximide (1.63 g, 88% yield).

LC-MS: RT=1.235 min, m/z=204.1 (M+1).

### Preparation of N-[4-[4-(pyrimidin-2-yl)piperazin-1-yl]-(4,4-²H₂)butan-2-yl]-2,3-endo-bicyclo[2.2.1]heptanedicarboximide

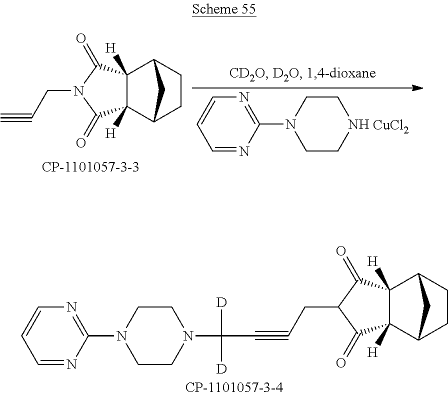

To a stirred solution of N-propargylbicyclo[2.2.1]heptane-2,3-dicarboximide (1.63 g, 8.03 mmol), 1-(2-pyrimidiny)piperazine (1.6 g, 9.64 mmol) and CD₂O (0.8 g, 24.09 mmol) in dioxane (20 ml) was added dropwise a solution of CuCl₂ (0.11 g, 0.803 mmol) in D₂O (10 ml) at room temperature. The reaction mixture was heated with stirring at 80° C. for overnight. After evaporation of the solvents, the residue was diluted with toluene; the insoluble materials were removed by filtration. The filtrated was washed with brine, and extracted with diluted HCl. The acidic extracts were neutralized with sat. NaHCO₃ solution and extracted with DCM. The combined DCM extracts were dried over Na₂SO₄, concentrated in vacuo to give N-[4-[4-(pyrimidin-2-yl)piperazin-1-yl]-(4,4-²H₂)butan-2-yl]-2,3-endo-bicyclo[2.2.1]heptanedicarboximide (1.32 g, 43.1% yield).

LC-MS: RT=0.953 min, m/z=382.1 (M+1).

### Preparation of (1R,2R,6S,7S)-4-{4-[4-(pyrimidin-2-yl)piperazin-1-yl]-(2,2,3,3,4,4-²H₆)butyl}-4-azatricyclo[5.2.1.0^{2,6}]decane-3,5-dione

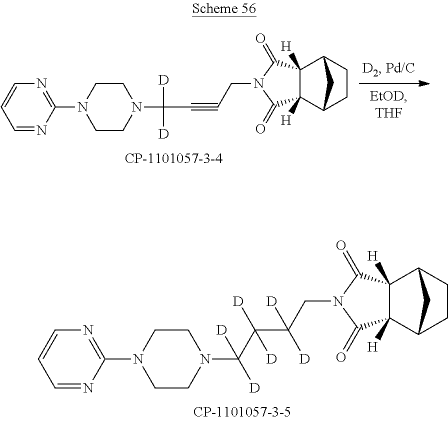 [SMILES: [2H]C([2H])(C#CCN1C(=O)[C@@H]2[C@@H]3CC[C@@H](C3)[C@@H]2C1=O)N1CCN(c2ncccn2)CC1 ; [2H]C([2H])(CN1C(=O)[C@@H]2[C@@H]3CC[C@@H](C3)[C@@H]2C1=O)C([2H])([2H])C([2H])([2H])N1CCN(c2ncccn2)CC1]

A solution of N-[4-[4-(pyrimidin-2-yl)piperazin-1-yl]-(4,4-²H₂)butyl-2-nyl]-2,3-endo-bicyclo[2.2.1]heptanedicarboximide (1.32 g, 3.46 mmol) in THF (50 ml) and EtOD (10 ml) was treated with Pd/C (0.13 g, 10% Pd on C) at 50° C. under 1 atm. of D₂ for overnight. The reaction mixture was filtered to remove the solid and the filtrate was concentrated in vacuo. The residue was purified by prep-HPLC to give N-[2,2,3,3,4,4-²H₆-4-[4-(pyrimidin-2-yl)piperazin-1-yl]-butyl]-2,3-endo-bicyclo[2.2.1]heptanedicarboximide (100 mg, 7.4% yield)

The end-product was analyzed to determine the melting point (Mp), LCMS retention time and mass, and the ¹H-NMR chemical shifts:

LC-MS: RT=1.017 min, m/z=390.2 (M+1)

HPLC: RT=4.092 min 96.85% (254 nm), 99.5% (214 nm)

¹H-NMR (400 MHz, CD₃OD): δ8.25 (d, 2H), 6.52 (t, 1H), 3.73 (t, 4H), 3.40 (s, 2H), 3.06 (m, 2H), 2.62 (s, 2H), 2.45 (t, 4H), 1.50˜1.63 (m, 4H), 1.15 (m, 2H).

### Preparation of N-[2,2,3,3,4,4-²H₆-4-[4-(pyrimidin-2-yl)piperazin-1-yl]-butyl]-2,3-endo-bicyclo[2.2.1]heptanedicarboximide; citric salt

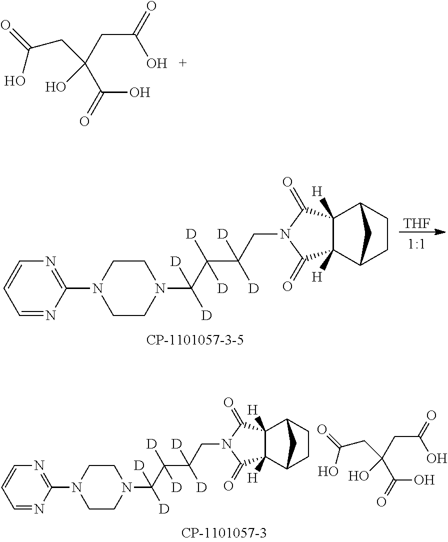

A solution of N-[2,2,3,3,4,4-²H₆-4-[4-(pyrimidin-2-yl)piperazin-1-yl]-butyl]-2,3-endo-bicyclo[2.2.1]heptanedicarboximide (210 mg, 0.54 mmol) and citric acid (113.4 mg, 0.54 mmol) in THF (50 ml) was stirred at room temperature for 2 hours, and then the reaction solution was let to stay for 2 days. The solid was separated by filtration. The collected solid was re-dissolved in water and then lyophilized.

The end-product was analyzed to determine the melting point (MP), LCMS retention time and mass, and the ¹H-NMR chemical shifts:

Mp: 59.0˜70.6° C.

LC-MS: RT=1.026 min, m/z=390.2 (M+1)

¹H-NMR (400 MHz, CD₃OD): 8.42 (d, 2H); 6.73 (t, 1H); 4.07 (br, 4H); 3.51 (s, 2H); 3.26 (br, 4H); 3.17 (s, 2H); 2.85˜2.89 (d, 2H); 2.75˜2.79 (d, 2H); 2.71 (s, 2H); 1.59˜1.66 (m, 4H); 1.22 (m, 2H)

### Example 17

### Synthesis of (1R,2R,6S,7S)-4-{4-[4-((5-²H)pyrimidin-2-yl)piperazin-1-yl]-(4,4-²H₂)butyl}-(2,6-²H₂)-4-azatricyclo[5.2.1.0^{2,6}]decane-3,5-dione

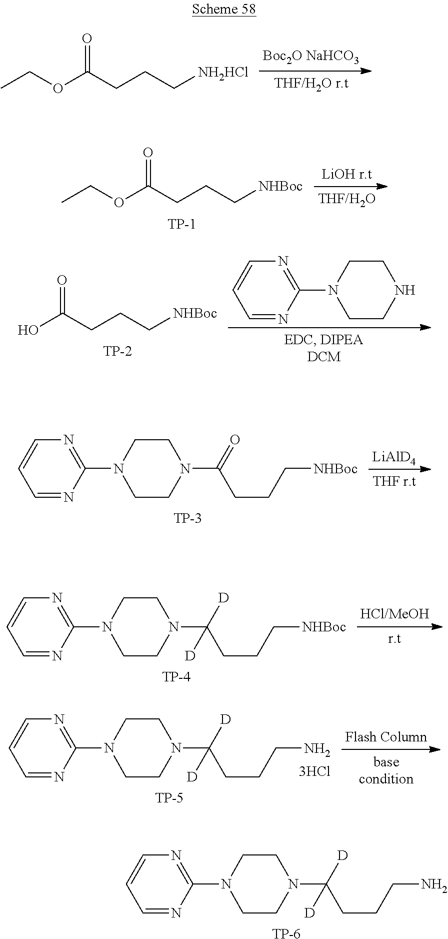

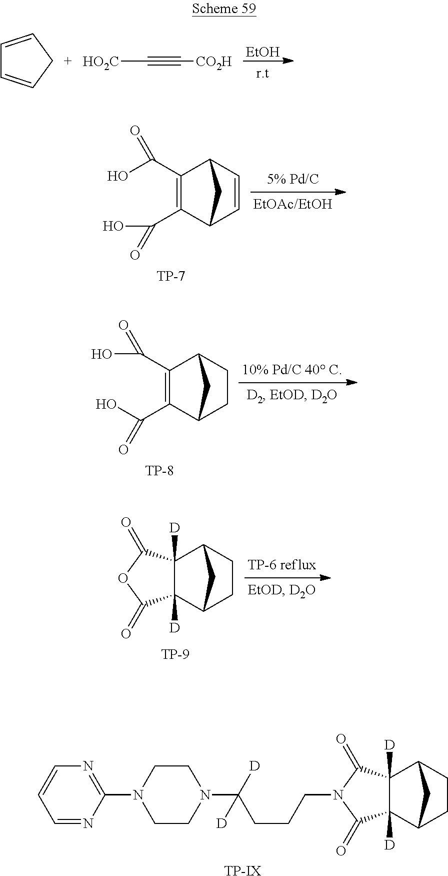

### Preparation of ethyl 4-(tert-butoxycarbonylamino)butanoate

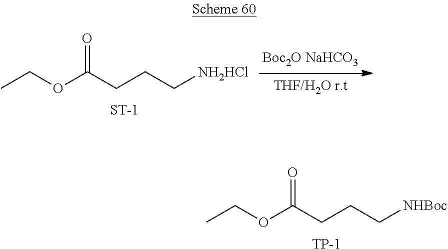

A solution of ethyl 4-aminobutanoate hydrochloride (3.0 g, 18 mmol), Boc₂O (3.91 g, 18 mmol) and K₂CO₃ (7.5 g, 54 mmol) in THF/H₂O (50 ml, 1:1) was stirred at room temperature for 4 hrs. The reaction mixture was diluted with water (100 ml), extracted with ethyl acetate (100 ml×2), the combined organic phase was washed with brine, dried over Na₂SO₄, concentrated in vacuo to give ethyl 4-(tert-butoxycarbonylamino) butanoate (3.6 g, 86.6% yield) as a white oil.

LCMS: t_{R}=1.304 min, m/z=118 (M−100)

### Preparation of 4-(tert-butoxycarbonylamino)butanoic acid

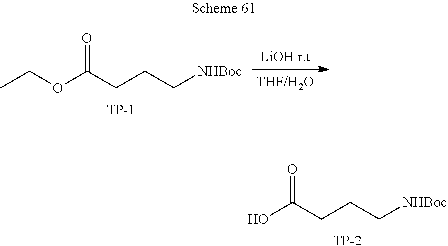

A suspension of ethyl 4-(tert-butoxycarbonylamino)butanoate (3.6 g, 15.58 mmol) and LiOHH₂O (1.96 g, 46.74 mmol) in THF/H₂O (60 ml, 1:1) was stirred at RT for 4 hrs. The reaction mixture was diluted with water (150 ml), extracted with Et₂O (100 ml×2). The aqueous was acidified with 2.0 N HCl to PH 3-4, and then extracted with ethyl acetate (150 ml×2), the combined organic phase was dried over Na₂SO₄, concentrated in vacuo to afford 4-(tert-butoxycarbonylamino)butanoic acid (4.5 g, 100%) as a white oil.

The end-product was analyzed to determine the melting point (Mp), LCMS retention time and mass, and the ¹H-NMR chemical shifts:

LCMS: t_{R}=1.376 min, m/z=226 (M+Na)

¹HNMR (400 MHz, CDCl3): δ: 12.05 (s, 1H); 6.85 (br, 1H); 2.94 (m, 2H); 2.21 (m, 2H); 1.60 (m, 2H); 1.38 (s, 9H).

### Preparation of tert-butyl 4-oxo-4-(4-(pyrimidin-2-yl)piperazin-1-yl) butylcarbamate

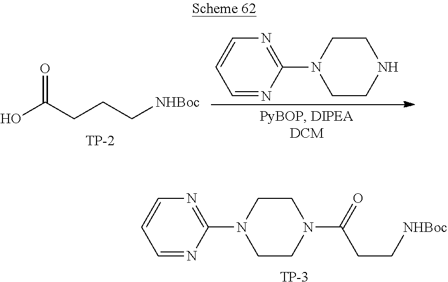

A solution of 4-(tert-butoxycarbonylamino)butanoic acid (4.54 g, 22.4 mmol), PyBOP (14.3 g, 27.4 mmol) and DIPEA (7.1 g, 54.9 mmol) in DCM (100 ml) was stirred at RT for 10 min, and then 2-(piperazin-1-yl)pyrimidine (3.0 g, 18.6 mmol) was added. The resulting mixture was stirred at RT for overnight. The reaction mixture was diluted with DCM (100 ml), washed with water (150 ml×2). The organic phase was dried over Na₂SO₄, concentrated in vacuo to give the crude product which was purified by combiflash column (100% EtOAc) to afford tert-butyl 4-oxo-4-(4-(pyrimidin-2-yl)piperazin-1-yl) butylcarbamate (3.57 g, 55.1% yield) as a white oil.

The end-product was analyzed to determine the LCMS retention time and mass, and the ¹H-NMR chemical shifts:

LCMS: t_{R}=1.23 min, m/z=350.1 (M+1).

¹HNMR (400 MHz, DMSO-d6): δ: 8.39 (d, 2H); 6.84 (m, 1H); 6.68 (m, 1H); 3.76 (m, 4H); 3.52 (m, 4H); 2.96 (m, 2H); 2.37 (M, 2H); 1.64 (m, 2H); 1.37 (s, 9H)

### Preparation of tert-butyl 4-(4-(pyrimidin-2-yl)piperazin-1-yl) (4,4-²H₂)butylcarbamate

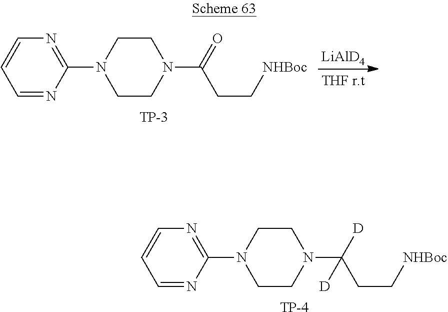

A solution of tert-butyl 4-oxo-4-(4-(pyrimidin-2-yl)piperazin-1-yl) butylcarbamate (0.5 g, 1.433 mmol) in THF (80 ml) was treated with LiAlD₄ (60.2 mg, 1.433 mmol) at 0° C.

And then the resulting mixture was stirred at RT for overnight. The reaction mixture was treated with Na₂SO₄.10H₂O for 1.5 hrs. And then filter to remove the solid, the filtrate was concentrated in vacuo to give tert-butyl 4-(4-(pyrimidin-2-yl)piperazin-1-yl) (4,4-²H₂)butylcarbamate (420 mg, 87% yield) as a colorless oil, which was used in next step without further purification.

LCMS: t_{R}=0.894 min m/z=338.2 (M+1)

### Preparation of 4-(4-(pyrimidin-2-yl)piperazin-1-yl)(4,4-²H₂)butan-1-amine

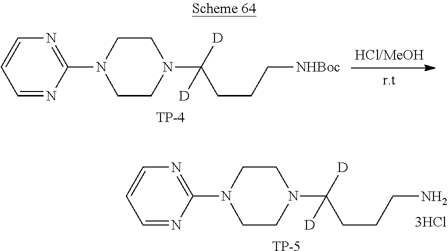

A solution of tert-butyl 4-(4-(pyrimidin-2-yl)piperazin-1-yl) (4,4-²H₂)butylcarbamate (420 mg, 1.246 mmol) in methanol (50 ml) was treated with 3.0 M HCl/MeOH (50 ml, 150 mmol) for 3 hrs. And then the reaction mixture was concentrated in vacuo to give 4-(4-(pyrimidin-2-yl) (4,4-²H₂)piperazin-1-yl) butan-1-amine hydrochloride (310 mg, 80.8 yield).

The hydrochloride salt was purified by flash column (base condition) to give the free amine TP-6.

The end-product was analyzed to determine the LCMS retention time and mass, and the ¹H-NMR chemical shifts:

LCMS: t_{R}=0.309 min m/z=238.2 (M+1).

¹HNMR (400 MHz, MeOD): δ: 8.73 (d, 2H); 7.17 (dd, 1H); 4.87 (m, 1H); 4.27 (m, 1H); 3.86 (m, 3H); 3.49 (m, 2H); 3.31 (m, 1H); 3.08 (m, 2H); 1.97 (m, 2H); 1.81 (m, 2H)

### Preparation of bicyclo[2.2.1]hepta-2,5-diene-2,3-dicarboxylic acid

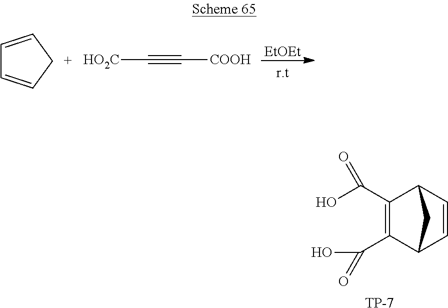

To a solution of but-2-ynedioic acid (5.0 g, 43.8 mmol) in ether (40 ml) was added dropwise 3.8 ml (45.6 mmol) of freshly distilled cyclopenta-1,3-diene at RT under a N₂ atmosphere. The reaction mixture, which warmed slightly after a short period of time, was stirred at RT Evaporation of the solvent in vacuo and trituration of the residue in petroleum ether, which was then crystallized in water to give bicyclo[2.2.1]hepta-2,5-diene-2,3-dicarboxylic acid (2.6 g, 40% yield) as a white solid.

LCMS: t_{R}=0.78 min m/z=181.0 (M+1).

### Preparation of bicyclo[2.2.1]hept-2-ene-2,3-dicarboxylic acid

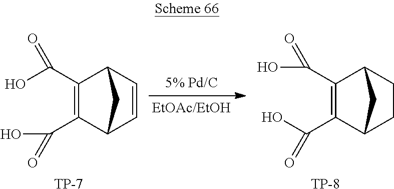

A solution of bicyclo[2.2.1]hepta-2,5-diene-2,3-dicarboxylic acid (2.5 g, 139 mmol) in EtOAc (36 ml) and EtOH (6 ml) was treated with Pd/C (250 mg) at RT under H₂ atmosphere for 3 hrs. The reaction mixture was filtered to remove the solid and filtrate was evaporated in vacuo to give bicyclo[2.2.1]hept-2-ene-2,3-dicarboxylic acid (2.6 g, 100% yield) as a white solid.

LCMS: t_{R}=0.863 min m/z=183.0 (M+1).

### Preparation of 2,3-²H₂-bicyclo[2.2.1]heptane-2,3-dicarboxylic acid

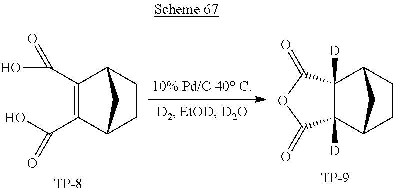

A solution of bicyclo[2.2.1]hept-2-ene-2,3-dicarboxylic acid (400 mg, 2.2 mmol) in EtOD (15 ml) and a few drops of D₂O was treated with Pd/C (40 mg, 10% treated with D₂O before used) under 1 atm of D₂ at 45° C. for 3 hrs. after filtrated to remove the solid, the reaction solution was used in next step without further purification.

LCMS: Rt=1.259 min m/z=169.0 (M+1).

### Preparation of (1R,2R,6S,7S)-4-{4-[4-((5-²H)pyrimidin-2-yl)piperazin-1-yl]-(4,4-²H₂)butyl}-(2,6-²H₂)-4-azatricyclo[5.2.1.0^{2,6}]decane-3,5-dione

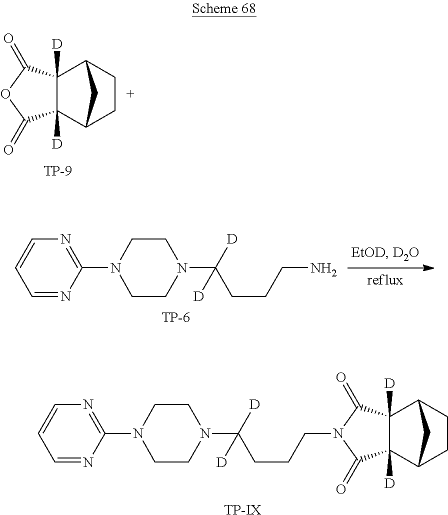

A solution of 2,3-²H₂-bicyclo[2.2.1]heptane-2,3-dicarboxylic acid (˜2.2 mmol) and 4-(4-(pyrimidin-2-yl)piperazin-1-yl)-(4,4-²H₂)butan-1-amine (564.3 mg, 2.38 mmol) in EtOD (50 ml) was heated to reflux for overnight. The reaction mixture was concentrated in vacuo to remove the solvent. The crude product was purified by prep-HPLC to give D-tandospirone (140 mg, 16.4%) as colourless oil.

The crude product was analyzed to determine the HPLC retention time, LCMS retention time and mass, and the ¹H-NMR chemical shifts:

LCMS: t_{R}=0.886 min m/z=388.2 (M+1)

HPLC: t_{R}=5.073 min 100% (214 nm, 254 nm)

¹H NMR (400 MHz, CDCl₃): δ 8.36 (d, 2H); 6.65 (t, 1H); 4.85 (m, 3H); 3.46˜3.67 (m, 5H); 2.74 (m, 3H); 2.63 (m, 1H); 1.56˜1.81 (m, 7H); 1.16˜1.26 (m, 3H).

### Example 19

### Metabolic Stability in Human Liver Microsomes

Elimination half-life of tandospirone is inverse proportional to the hepatic clearance rate. Intrinsic clearance rate has been determined for 6 deuterated compounds in human liver microsomes, using tandospirone as comparison, Testosterone (a CYP3A4 substrate), Propafenone (CYP2D6 substrate) and Diclofenac (CYP2C9 substrate) as reference.

Test compounds (at 1 μM) was incubated at 37° C. in a 100 mM potassium phosphate buffer, pH 7.4, 10 mM MgCl₂, with pooled human liver microsomes (Cat No. H0630, Lot No. 0910398, Xenotech) at 0.7 mg/mL microsomal protein in a 96-well format. The mixture of compound and microsome was pre-incubated for 30 minutes, initiating the reaction by addition of NADPH regenerating system (1 unit/mL Isocitric dehydrogenase). At time point 0, 5, 15, 30, 45 and 60 minutes the reactions were stopped by addition of 300 μL cold acetonitrile (40° C.) containing 100 ng/mL tolbutamide as internal standard (IS). The samples were immediately mixed and centrifuged at 4000 rpm for 20 min. 100 μL supernatant was transferred to a fresh 96-well containing 300 μL HPLC-grade water for quantitative analysis by LC-MS/MS (LC: Shimatzu LC 10 AD; MS/MS detection: Sciex API4000), using positive ion electrospray. Triplicate experiments (n=3) was performed for each compound. A single-exponential curve (Cₜ=C₀*e^{-kt}) was fitted to the concentration-time data points to calculate the elimination rate constant (k). The following equation 1 was used to calculate the total hepatic intrinsic clearance (CLint):

$${CL}_{int} = k \cdot \frac{1}{0.7 mg \text{/} ml microsomalproteinin incubation} \cdot \frac{45 mg microsomes}{g liver} \cdot \frac{22 g liver}{kg body weight} \qquad Equation 1$$

The calculated CLint are collected and shown in Table V below. The tested compounds of the present invention (Compound II, III, VI, VII, XV and XVIII of tables II and III) all displayed a statistically significant lower intrinsic clearance compared to tandospirone (P<0.05; One-way ANOVA followed by Dunnett's Multiple Comparison Test vs Tandospirone).

TABLE V
Mean hepatic intrinsic clearance (CLint) with standard error of mean
(sem) of new deuteriated analogues compared to tandospirone in
human liver microsomes following incubation of 1 μM test compound
(n = 3). Testosterone, Diclofenac and propafenone are included
as positive controls for CYP3A4, CYP2C9 and CYP2D6 activity,
respectively.

| Compound | CLint (mL/min/kg) | sem |
|---|---|---|
| III | 219** | 4.4 |
| VII | 231* | 3.4 |
| XVIII | 224** | 7.6 |
| II | 235* | 4.1 |
| VI | 240* | 4.3 |
| XV | 229** | 6.0 |
| Tandospirone | 282 | 23 |
| Testosterone | 70 | 1.3 |
| Diclofenac | 90 | 0.42 |
| Propafenone | 202 | 2.4 |

*P < 0.05;
**P < 0.01, One-way ANOVA with Dunnett's Multiple Comparison Test vs Tandospirone

This study demonstrates an increased metabolic stability by the substitution of hydrogen with deuterium for the tested compounds, which will likely contribute to a longer plasma half-life for the new deuterated analogues compared to tandospirone.

### Example 20

### Plasma Protein Binding

Binding to human plasma proteins have been evaluated for six deuterated tandospirone compounds in an equilibrium dialysis assay.

Human plasma was obtained from Bioreclamation (Lot No. BRH354026). A 96-Well Micro-equilibrium Dialysis Device (HT-Dialysis LLC, Model HTD 96 b) separated by dialysis membrane (MWCO 12-14k, HT Dialysis), was used to study the protein binding. The dialysis membrane strips were soaked in distilled water for 1 hour and then in 20% ethanol in water (v/v) for another 20 minutes. The membrane strips were subsequently rinsed 20 min in pure water before use. The studied compounds were individually spiked into human plasma to a final concentration of 0.2 μM, and pre-incubated for 30 min. Aliquots (150 μL) of phosphate buffer (100 mM sodium phosphate, pH 7.4) and spiked plasma was placed in the receiver and donor compartment, respectively. The dialysis block was sealed and placed in a shaker (150 rpm) for 6 hours at 37° C. Each experiment was performed in triplicates (n=3). Aliquot 50 μL from both the donor sides and receiver sides of the dialysis devise was transferred into new sample preparation plates and mixed with the aliquots with same volume of opposite matrixes (blank buffer to plasma and visa verse). The samples were subsequently quenched with 200 μL acetonitrile (ACN) containing internal standard (tolbutamide), vortexed at 800 rpm for 20 min followed by centrifugation at 3220 RCF for 20 min. Hundred μL of the supernatant was transferred to a new 96-well plate and mixed with 200 μL of Milli-Q water containing 0.1% FA. Drug content was quantified with LC/MS/MS (Sciex API4000).

Percent bound to plasma is calculated as follows:

% Bound=100−(100×[Receiver]₆ₕ/[Donor]₆ₕ.) Equation 2

Mass recovery of test compound from the HTDialysis after 6 h is calculated as follows:

% Recovery=100×([Donor]₆ₕ+[Receiver]₆ₕ)/[Donor]₀ₕ Equation 3

Results are collected in Table VI. The observed unbound fraction of tandospirone (30%) lies within that reported in the literature (30.4%) (Miller et al., 1992). Compounds II, III, VI, VII, XV and XVIII of tables II and Ill were tested. Five (II, III, VI, IX and XVIII) out of the six tested compounds of the current invention possessed statistically significant lower degree of protein binding in human plasma. The higher unbound fraction of compound II, III, VI, XV and XVIII, may in vivo contribute to higher diffusion rate into the target organ (such as the brain), in addition to higher availability of unbound drug for interaction with the target receptors for example within the CNS.

TABLE VI
Mean protein binding in human plasma, following 5 h dialysis equilibrium
with selected test compounds of this invention, compared to tandospirone.
Wafarin is included as reference control.

| | Protein binding | | | |
|---|---|---|---|---|
| Compound | (%) | sem | Recovery (%) | sem |
| III | 49*** | 0.42 | 86 | 3.1 |
| VII | 69 | 1.8 | 94 | 2.4 |
| XVIII | 53*** | 3.6 | 111 | 7.1 |
| II | 58** | 0.12 | 95 | 1.7 |
| VI | 57** | 0.058 | 96 | 2.3 |
| XV | 62* | 0.44 | 92 | 0.75 |
| Tandospirone | 70 | 2.8 | 82 | 3.9 |
| Warfarin | 99 | 0.067 | 89 | 1.9 |

*P < 0.05;
**P < 0.01:
***P < 0.001, One-way ANOVA followed by Dunnett's Multiple Comparison Test vs Tandospirone

### Example 21

### Permeability Over MDR1-Transfected MDCK Cell Monolayer

The ability of molecules to pass a biological membrane, like the gastro-intestinal epithelia and the blood-brain barrier endothelia is essential for orally delivered drugs targeting diseases in the brain. The Madin-Darby Canine Kidney (MDCK) cell line is used as an industry standard to evaluate biomembrane passage properties of drugs. MDCK-MDR1 cells (obtained from Piet Borst at the Netherlands Cancer Institute; passage 15) were seeded onto polyethylene membranes (PET) in 96-well BD insert systems at 2×105 cells/cm2 until to 4-6 days for confluent cell monolayer formation. Test compounds were diluted with the transport buffer (HBSS+1% BSA) from a 10 mM stock solution to a concentration of 2 μM and applied to the apical (A) or basolateral (B) side of the cell monolayer.

Permeation of the test compounds from A to B direction or B to A direction was determined in triplicate over a 150-minute incubation at 37° C. and 5% CO₂ with a relative humidity of 95%. In addition, the efflux ratio of each compound was also determined. Test and reference compounds were quantified by LC-MS/MS analysis (LC: Shimatzu LC 10 AD; MS/MS detection: Sciex API4000) based on the peak area ratio of analyte/IS (tolbutamide 250 ng/ml).

The apparent biomembrane permeability coefficient Papp (cm/s) was calculated using the equation:

Papp=(dCr/dt)×Vr/(A×C₀) Equation 4

Where dCr/dt is the cumulative concentration of compound in the receiver chamber as a function of time (μM/s); Vr is the solution volume in the receiver chamber (0.075 mL on the apical side, 0.25 mL on the basolateral side); A is the surface area for the transport, i.e. 0.084 cm2 for the area of the monolayer; Co is the initial concentration in the donor chamber.

The efflux ratio was calculated using the equation:

Efflux Ratio=Papp(BA)/Papp(AB) Equation 5

A B-A/A-B efflux ratio>2 indicates that the compound may be a substrate for an efflux transporter like P-gp.

Percent recovery was calculated using the equation:

% Recovery=100×[(Vr×Cr)+(Vd×Cd)]/(Vd×C0) Equation 6

Where Vd is the volume in the donor chambers (0.075 mL on the apical side, 0.25 mL on the basolateral side); Cd and Cr are the final concentrations of transport compound in donor and receiver chambers, respectively.

Results from the permeability study are presented in Table VII. Compound III (of Table II) displays statistically significant higher Pₐₚₚ, A-B compared to tandospirone (P<0.05; One-way ANOVA followed by Dunnett's Multiple Comparison Test). Thus, suggesting that compound III may have improved absorption in the gastro-intestinal tract following oral administration in addition to higher passage rate into the brain.

TABLE VII
Mean apparent biomembrane permeability (Pₐₚₚ) of deuterated
tandospirone compound III of Table II compared to tandospirone over a
confluent MDR1-MDCK cell monolayer (2 μM, n = 3). Recovery
in the donor and receiver chamber is displayed in addition to efflux ratio
(B-A/A-B Pₐₚₚ) Fenoterol, propanolol and digoxin (n = 2) are used
as reference compounds for MDR1-MDCK confluency/functionality.

| | | | Pₐₚₚ, A-B | | B-A/A-B | |
|---|---|---|---|---|---|---|
| | Recovery | | (×10⁻⁶ | | efflux | |
| Compound | (%) | sem | cm/s) | sem | ratio | sem |
| III | 112 | 2.3 | 35* | 0.48 | 0.8 | 0.02 |
| Tandospirone | 107 | 0.60 | 30 | 0.87 | 0.9 | 0.03 |
| Fenoterol | 109 | 1.1 | 0.92 | 0.08 | nd | |
| Propranolol | 84 | 1.7 | 15 | 0.28 | 62 | 2.0 |
| Digoxin | 98 | 5.1 | 0.19 | 0.02 | nd | |

*P < 0.05, One-way ANOVA with Dunnett's Multiple Comparison Test vs Tandospirone
nd: not determined

### Example 22

### Inhibition of Cytochrome P 3A4

Inhibition of the main drug metabolizing enzymes in human liver may lead to clinically significant drug-drug interactions. If two drugs are given in combination and are metabolised by the same enzymes, competition for metabolism may give rise to increased plasma concentrations and therefore possible adverse effects (Lin et al., 1997). The inhibitory potential of tandospirone and the novel structures in this invention have been tested towards the cytochrome P450 enzyme, CYP3A4, that is most frequently associated with drug metabolism and constitute the quantitative majority of P450 enzymes in the human liver (Shimada et al., 1994). CYP3A4 is the major enzyme involved in tandospirone metabolism (Niwa et al., 2005).

The inhibition potential of tandospirone and compound XI on the cytochrome P450 isoform, CYP3A4, was evaluated in recombinant CYP3A4 supersomes (BD Gentest, Cat No. 456202, Lot No. 48844). Midazolam (2 μM) was used as substrate, monitoring the formation of the 1′-hydroxymidazolam metabolite by LC-MS/MS analysis (LC: Shimatzu LC 10 AD; MS/MS detection: Sciex API4000).

The incubation was performed with a 5 μmol CYP supersome and 2 μM midazolam solution (100 μL) in a potassium phosphate buffer (100 mM, pH 7.4), followed by the addition of 2 μL test compound (0, 1, 10, 50 or 100 μM) into a 96-well plate. The reaction was subsequently initiated by the addition of 98 μL cofactor mixture (3.14 mM MgCl₂, 2.82 mM G6P, 1.25 mM NADP in phosphate buffer). The incubation was terminated after 3 min by the addition of 200 μL IS-fortified stop solution (0.2 μM 1′-Hydroxymidazolam-[¹³C₃] in 97% ACN, 3% FA). The plate was centrifuged at 4000 rpm for 20 min and 100 μL supernatant removed and mixed with 300 μL HPLC-grade water for quantitative analysis.

IC₅₀ were calculated from curve fit of the following equation 7 to the 1′-hydroxymidazolam elimination rate relative to control vs inhibitor concentration, where min and max is defined as the respectively minimum and maximum values of % of control activity in curve:

$$y = min + \frac{max - min}{1 + {( \frac{x}{IC 50} )}^{- Hillslope}} \qquad Equation 7$$

The IC₅₀ for CYP3A4 inhibition of the deuterated tandospirone compound XV of Table II in addition to tandospirone is reported in Table VIII. A statistically significant higher IC₅₀ was found for compound XV compared to the non-deuterated tandospirone (P<0.05, One-way ANOVA followed by Dunnett's Multiple Comparison Test vs Tandospirone). Therefore, Compound XV may have lower propensity for drug-drug interaction with drugs metabolised by the cytochrome P 3A4 isoform.

TABLE VIII
Inhibition of CYP3A4-mediated metabolism of midazolam, monitoring
transition to 1′-hydroxymidazolam. IC₅₀ estimated from the compound
XV concentrations between 1-100 μM (n = 3) using ketoconazole
as positive control

| Compound | CYP3A4 IC₅₀ (μM) | sem |
|---|---|---|
| XV | 39* | 2.1 |
| Tandospirone | 27 | 2.9 |
| Ketoconazole | 0.013 | 0.0007 |

*P < 0.05, One-way ANOVA with Dunnett's Multiple Comparison Test vs Tandospirone

### REFERENCES

1. Hamik; Oksenberg, D; Fischette, C; Peroutka, S J (1990. Biological psychiatry 28 (2): 99-109.
2. Tanaka; Tatsuno, T; Shimizu, H; Hirose, A; Kumasaka, Y; Nakamura, M (1995). General pharmacology 26 (8): 1765-72.
3. Yabuuchi, Kazuki; Tagashira, Rie; Ohno, Yukihiro (2004). Biogenic Amines 18: 319.
4. Blier; Curet, O; Chaput, Y; De Montigny, C (1991). Neuropharmacology 30 (7): 691-701.
5. Miller; Thompson, M L; Byrnes, J J; Greenblatt, D J; Shemer, A (1992). Journal of clinical psychopharmacology 12 (5): 341-5.
6. Natsui et al: Eur J Drug Metab Pharmacokinet. 2007; 32(3):131-7
7. Nishikawa et al: Prog Neuropsychopharmacol Biol Psychiatry. 2007; 31(4):926-31;
8. Nishikawa et al: Psychiatry Clin Neurosci. 2008; 62(5):591-6.
9. Gannes L Z et al., Comp. Biochem Physiol Mol Integr Physiol. 1998, 119: 725
10. Remington: The Science and Practice of Pharmacy, 20^{th} Edition, Gennaro, Ed., Mack Publishing Co., Easton, Pa., 2000.
11. Berge et al: J. Pharm. Sci. 1977, 66, 2
12. Ishizumi, K.; Kojima, A.; Antoku, F. Chem Pharm Bull. 1991, 39(9): 2288.
13. Suzuki et al. (1999) Drug Metab. Dispos., 27: 1254-1259
14. Walter et al., Dug Metab Dispos. 2003; 31(6):714-7
15. Konsoula et al: Int JPharm. 2008; 361(1-2): 19-25
16. Lin J H, Lu A Y (1997). Pharmacol Rev. 49(4): 403-49
17. Lin F (2011). Anal Bioanal Chem 400:2881-2887.
18. Miller L G, Thompson M L, Byrnes J J, Greenblatt D J, Shemer A (1992). J Clin Psychopharmacol. 12(5):341-5.
19. Nakashima M, Kanemaru M (1992). Kiso to Rinsh 26:4143-4165.
20. Niwa T, Shiraga T, Ishii I, Kagayama A, Takagi A (2005). Biol Pharm Bull. 28(9):1711-1716
21. Shimada T, et al. (1994). J Pharmacol Exp Ther 270(1): 414-23

1.-29. (canceled)

30. A deuterated compound of the Formula I 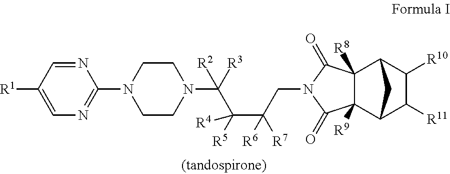
wherein R1, R4, R5, R6, R7, R8, R9, R10 and are individually selected from the group consisting of hydrogen (H) and deuterium (D) and one or both of positions R2 and R3 are deuterium.

31. The compound according to claim 30 wherein both of positions R2 and R3 are deuterium (D) and the positions R1, R4, R5, R6, R7, R8, R9, R10 and R11 are individually selected from deuterium (D) or hydrogen (H).

32. The compound according to the claim 30, wherein R2 is deuterium (D), R3 is hydrogen (H), and the positions R1, R4, R5, R6, R7, R8, R9, R10 and R11 are individually selected from deuterium (D) or hydrogen (H).

33. The compound according to the claim 30, wherein R3 is deuterium (D), R2 is hydrogen (H), and the positions R1, R4, R5, R6, R7, R8, R9, R10 and R11 are individually selected from deuterium (D) or hydrogen (H)

34. The compound according to claim 30, wherein one or both of positions R2 and R3 are deuterium (D) and the positions R1, R4, R5, R6, R7, R8, R9, R10 and R11 are hydrogen (H).

35. The compound according to claim 30, wherein said compound is selected from the group consisting of:
2R,6S,7S)-4-{4-[4-(pyrimidin-2-yl)piperazin-1-yl]-(4,4-²H₂)butyl}-4-azatricyclo-[5.2.1.0^{2,6}]decane-3,5-dione (II),
(1R,2R,6S,7S)-4-{4-[4-((5-²H)pyrimidin-2-yl)piperazin-1-yl]-(4,4-²H₂)butyl}-4-azatricyclo[5.2.1.0^{2,6}]decane-3,5-dione (III),
(1R,2R,6S,7S)-4-{4-[4-(pyrimidin-2-yl)piperazin-1-yl]-(4,4-²H₂)butyl}-(8,9-²H₂)-4-azatricyclo[5.2.1.0^{2,6}]decane-3,5-dione (VI),
(1R,2R,6S,7S)-4-{4-[4-(((5-²H)pyrimidin-2-yl)piperazin-1-yl]-(4,4-²H₂)butyl}-(2,6-H₂)-4-azatricyclo[5.2.1.0^{2,6}]decane-3,5-dione (VII),
(1R,2R,6S,7S)-4-{4-[4-(pyrimidin-2-yl)piperazin-1-yl]-(4-²H₂)butyl}-(2,6-²H₂)-4-azatricyclo[5.2.1.0^{2,6}]decane-3,5-dione (XV),
(1R,2R,6S,7S)-4-{4-[4-(pyrimidin-2-yl)piperazin-1-yl]-(2,2,3,3,4,4-²H₆)butyl}-4-azatricyclo[5.2.1.0^{2,6}]decane-3,5-dione (XVIII),
(1R,2R,6S,7S)-4-{4-[4-((5-²H)pyrimidin-2-yl)piperazin-1-yl]-(4,4-²H₂)butyl}-(2,6-²H₂)-4-azatricyclo[5.2.1.0^{2,6}]decane-3,5-dione,
(1R,2R,6S,7S)-4-{4-[4-((5-²H)pyrimidin-2-yl)piperazin-1-yl]-(2,2,3,3,4,4-²H₆)butyl}-4-azatricyclo[5.2.1.0^{2,6}]decane-3,5-dione,
(1R,2R,6S,7S)-4-{4-[4-((5-²H)pyrimidin-2-yl)piperazin-1-yl]-(2,2,3,3,4,4-²H₆)butyl}-(2,6-²H₂)-4-azatricyclo[5.2.1.0^{2,6}]decane-3,5-dione,
(1R,2R,6S,7S)-4-{4-[4-(pyrimidin-2-yl)piperazin-1-yl]-(2,2,3,3,4,4-²H₆)butyl}-(2,6-²H₂)-4-azatricyclo[5.2.1.0^{2,6}]decane-3,5-dione,
(1R,2R,6S,7S)-4-{4-[4-(pyrimidin-2-yl)piperazin-1-yl]-(2,2,3,3,4,4-²H₆)butyl}-(8,9-²H₂)-4-azatricyclo[5.2.1.0^{2,6}]decane-3,5-dione,
(1R,2R,6S,7S)-4-{4-[4-((5-²H)pyrimidin-2-yl)piperazin-1-yl]-(2,2,3,3,4,4-²H₆)butyl}-(8,9-H₂)-4-azatricyclo[5.2.1.0^{2,6}]decane-3,5-dione,
(1R,2R,6S,7S)-4-{4-[4-((5-²H)pyrimidin-2-yl)piperazin-1-yl]-(2,2,3,3,4,4-²H₆)butyl}-4-azatricyclo[5.2.1.0^{2,6}]decane-3,5-dione,
(1R,2R,6S,7S)-4-{4-[4-((5-²H)pyrimidin-2-yl)piperazin-1-yl]-(2,2,3,3,4,4-²H₆)butyl}-(2,6-²H₂)—4-azatricyclo[5.2.1.0^{2,6}]decane-3,5-dione,
(1R,2R,6S,7S)-4-{4-[4-((5-²H)pyrimidin-2-yl)piperazin-1-yl]-(2,2,3,3,4,4-²H₆)butyl}-(2,6-²H₂)— (8,9-²H₂)-4-azatricyclo[5.2.1.0^{2,6}]decane-3,5-dione,
(1R,2R,6S,7S)-4-{4-[4-(pyrimidin-2-yl)piperazin-1-yl]-(2,2,3,3,4,4-²H₆)butyl}-(2,6-²H₂-(8,9-²H₂)-4-azatricyclo[5.2.1.0^{2,6}]decane-3,5-dione,
(1R,2R,6S,7S)-4-{4-[4-((5-²H)pyrimidin-2-yl)piperazin-1-yl]-(4,4-²H₂)butyl}-(2,6-²H₂)-4-azatricyclo[5.2.1.0^{2,6}]decane-3,5-dione,
(1R,2R,6S,7S)-4-{4-[4-((5-²H)pyrimidin-2-yl)piperazin-1-yl]-(4,4-²H₂)butyl}-(2,6-²H₂)— (8,9-²H₂)-4-azatricyclo[5.2.1.0^{2,6}]decane-3,5-dione, and
(1R,2R,6S,7S)-4-{4-[4-(pyrimidin-2-yl)piperazin-1-yl]-(4,4-²H₂)butyl}-(2,6-²H₂)-(8,9-²H₂)-4-azatricyclo[5.2.1.0^{2,6}]decane-3,5-dione.

36. A pharmaceutical composition comprising a compound according to claim 30.

37. A method for treatment of diseases or conditions wherein activation of the serotonin 5-HT1A receptor has a beneficial therapeutic effect, or for treatment of diseases associated with dysfunction of the serotonin 5-HT1A receptor, said method comprising administering an effective amount of a compound according to claim 30 to an individual in need thereof.

38. A method for treatment of dermatological disorders, wherein said dermatological disorder may be selected from the group consisting of atopic dermatitis, seborrhoeic dermatitis, diaper dermatitis, allergic contact dermatitis, irritant contact dermatitis, unspecified contact dermatitis, infective dermatitis, exfoliative dermatitis, lichen simplex chronicus, lichen planus, pruritus/itch, pityriasis rosea, rosacea, psoriasis, urticaria (allergic and unspecified), erythema, sunburn, pemphigus and other acantholytic disorders, dermatological disorders associated with stress and dermatological disorders associated with diseases of the central nervous system such as anxiety and depressions, said method comprising administering an effective amount of a compound according to claim 30 to an individual in need thereof.

39. A method for treatment of a condition selected from the group consisting of disorders of the central nervous system, cognitive impairment/dysfunction disorders, eating disorders, dyspepsia, treatment of development of tolerance to the treatment effects of morphine, opiates and alcohol, treatment of dependence of alcohol or tobacco smoking, treatment of dyspepsia, acute, chronic or idiopathic cough, age related macular degeneration (AMD) and sexual dysfunction, impairments, or dysfunctions caused by cerebral ischemia, movement disorders, pain and postoperative nausea and vomiting (PONV), said method comprising administering an effective amount of a compound according to claim 30 to an individual in need thereof.

40. The pharmaceutical composition according to claim 36, said composition further comprising one or more second active ingredient(s).

41. The pharmaceutical composition according to claim 40, wherein the second active ingredient is selected from the group consisting of serotonin reuptake inhibitors, corticosteroids, antibiotics, antihistamines, immunomodulators, vitamin derivatives and biologics.

42. The pharmaceutical composition according to claim 40, wherein the second active ingredient is an analgesic selected from the group consisting of analgesic medication classes including NSAIDs, COX-2 inhibitors, acetaminophen, other anti-inflammatory, tricyclic antidepressants, anticonvulsant agents, voltage gated calcium channel blockers, N-type calcium channel blockers, other calcium channel modulators, SNRI and other monoamine reuptake inhibitors, sodium channel blockers, NMDA antagonists, AMPA antagonists, other glutamate modulators, GABA modulators, CRMP-2 modulators, NK-1 antagonists, TRPV1 agonists, cannabinoids, adenosine agonists, nicotinic agonists, p38 MAP kinase inhibitors, corticosteroids, triptans used for treatment and prevention of migraine, and strong and weak opioids such as fentanyl, oxycodone, codeine, dihydrocodeine, hydrocodone, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papvereturn, alfentanil, buprenorphine and tramadol, and other analgesic drug classes

43. The pharmaceutical composition according to claim 40, wherein the second active ingredient is an opioid, wherein said opioid may be selected from the group consisting of hydrocodone, oxycodone, codeine or tramadol.

44. The pharmaceutical composition according to claim 40, wherein the second active ingredient is an antiemetic agents, wherein said antiemetic agent may be selected from the group consisting of 5-HT3 receptor antagonists, NK-1 antagonists, dopamine antagonists, H1 histamine receptor antagonists, cannabinoids, benzodiazepines, anticholinergic compounds and steroid compounds.

45. The compound according to claim 30, wherein said compound is to be administered by oral, rectal, nasal, pulmonary, buccal, sublingual, transdermal or parenteral administration.

46. The compound according to claim 30 for administering to a patient in need thereof in a therapeutically effective amount from 0.001 to 1000 mg.

47. The compound according to claim 46, wherein the therapeutically effective amount is from 0.01 to 600 mg.

48. The compound according to claim 46, wherein the therapeutically effective amount is from 0.5 mg to 200 mg.

49. A method for synthesis of a deuterated compound according to Formula I: 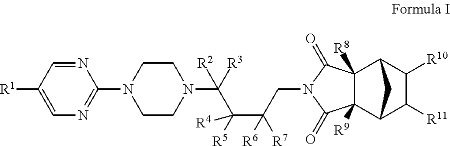
wherein R1, R4, R5, R6, R7, R8, R9, R10 and R11 are individually selected from the group consisting of hydrogen (H) and deuterium (D) and one or both of positions R2 and R3 are deuterium, and wherein said method comprises one or more of the following steps:
a) treating a mixture of reagent and 50% water-containing 10% palladium on charcoal in tetrahydrofuran (THF) by using D₂ gas,
b) stirring a solution of reagents and formalin-D₂ in dioxane and further adding drop wise a solution of copper sulphate in D₂O to form a mixture, subsequently stirring mixture, followed by concentration in vacuo and treatment with toluene to obtain a product which is further filtered and concentrated in vacuo,
c) hydrogenation over 10% palladium on charcoal of a reagent to give a mixture, subsequently filtering said mixture and concentrating said filtrate in vacuo to give a product,
d) mixing reagents by stirring a mixture with anhydrous K₂CO₃ and KI in anhydrous DMF, followed by stirring, cooling to room temperature, pouring mixture into water and separating the product in the organic phase using EtOAc, subsequently drying said organic phase and concentrating said product in vacuo,
e) refluxing a mixture of reagent, dibromobutane and anhydrous K₂CO₃ in acetone prior to cooling and filtration, subsequently concentrating filtrate in vacuo,
f) refluxing a mixture of reagents and pyridine, followed by cooling, and concentration of the obtained product in vacuo,
g) refluxing a mixture of reagent, propagyl bromide and anhydrous K₂CO₃ in anhydrous acetone under nitrogen, followed by cooling and filtertration, subsequent concentration of filtrate in vacuo and recrystallization from n-hexane to give a product,
wherein said reagent is selected from the group consisting of 1-(5-bromopyrimidin-2-yl)piperazin, (1R,2S,6R,7S)-4-(4-bromobutyl)-4-azatricyclo[5.2.1.0^{2,6}]decane-3,5-dione, 1-((5-²H)pyrimidin-2-yl)piperazin, (1R,2R,6S,7S)-4-{4-[4-(5-bromopyrimidin-2-yl)piperazin-1-yl]butyl}-4-azatricyclo[5.2.1.0^{2,6}]decane-3,5-dione, (1R,2S,6R,7S)-4-(prop-2-yn-1-yl)-4-azatricyclo[5.2.1.0^{2,6}]decane-3,5-dione, (1R,2S,6R,7S)-4-{4-[4-(pyrimidin-2-yl)piperazin-1-yl]-(4-²H₂)but-2-yn-1-yl}-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]decane-3,5-dione, 4-oxatricyclo[5.2.1.0^{2,6}]dec-8-ene-3,5-dione, 4-[4-(pyrimidin-2-yl)piperazin-1-yl]-4-butan-1-amine, (8,9-²H₂)-4-oxatricyclo[5.2.1.0^{2,6}]decane-3,5-dione, 4-[4-(5-bromopyrimidin-2-yl)piperazin-1-yl]-4-butan-1-amine, (8,9-²H₂)-4-azatricyclo[5.2.1.0^{2,6}]decane-3,5-dione, (1R,2R,6S,7S)-4-{4-[4-(5-bromopyrimidin-2-yl)piperazin-1-yl]butyl}-(8,9-²H₂)-4-azatricyclo[5.2.1.0^{2,6}]decane-3,5-dione, (1R,2S,6R,7S)-4-azatricyclo[5.2.1.0^{2,6}]dec-8-ene-3,5-dione, 1-(5-bromopyrimidin-2-yl)piperazin, (1R,2S,6R,7S)-4-(4-bromobutyl)-4-azatricyclo[5.2.1.0^{2,6}]dec-8-ene-3,5-dione, (1R,2R,6S,7S)-4-{4-[4-(5-bromopyrimidin-2-yl)piperazin-1-yl]-(4-²H₂)butyl}-(8,9-²H₂)-4-azatricyclo[5.2.1.0^{2,6}]decane-3,5-dione, (1R,2S,6R,7S)-4-(prop-2-yn-1-yl)-4-azatricyclo[5.2.1.0^{2,6}]dec-8-ene-3,5-dione, of (1R,2S,6R,7S)-4-{4-[4-(pyrimidin-2-yl)piperazin-1-yl]-(4-²H₂)but-2-yn-1-yl}-4-azatricyclo[5.2.1.0^{2,6}]dec-8-ene-3,5-dione, (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione, (1R,2S,6R,7S)-4-azatricyclo[5.2.1.0^{2,6}]dec-8-ene-3,5-dione, (8,9-²H₂)-4-azatricyclo[5.2.1.0^{2,6}]decane-3,5-dione, (1R,2S,6R,7S)-4-(prop-2-yn-1-yl)-4(8,9-²H₂)-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione, (1R,2S,6R,7S)-4-(prop-2-yn-1-yl)-4-azatricyclo[5.2.1.0^{2,6}]dec-8-ene-3,5-dione, 1-((5-bromopyrimidin-2-yl)piperazin, (1R,2S,6R,7S)-4-{4-[4-(5-bromopyrimidin-2-yl)piperazin-1-yl]-(4-²H₂)but-2-yn-1-yl}-4-azatricyclo[5.2.1.0^{2,6}]dec-8-ene-3,5-dione, (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione, and
wherein said product is selected from the group consisting of 4-((5-²H)pyrimidin-2-yl)piperazin, (1R,2R,6S,7S)-4-{4-[4-((5-²H)pyrimidin-2-yl)piperazin-1-yl]butyl}-4-azatricyclo[5.2.1.0^{2,6}]decane-3,5-dione, (1R,2S,6R,7S)-4-{4-[4-(pyrimidin-2-yl)piperazin-1-yl]-(4-²H₂)but-2-yn-1-yl}-4-azatricyclo[5.2.1.0^{2,6}]decane-3,5-dione, (1R,2R,6S,7S)-4-{4-[4-(pyrimidin-2-yl)piperazin-1-yl]-(4-²H₂)butyl}-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]decane-3,5-dione, ((1R,2R,6S,7S)-4-{4-[4-((5-²H)pyrimidin-2-yl)piperazin-1-yl]-(4-²H₂)butyl}-4-azatricyclo[5.2.1.0^{2,6}]decane-3,5-dione, (8,9-²H₂)-4-oxatricyclo[5.2.1.0^{2,6}]-decane-3,5-dione, (1R,2R,6S,7S)-4-{4-[4-(pyrimidin-2-yl)piperazin-1-yl]butyl}-(8,9-²H₂)-4-azatricyclo[5.2.1.0^{2,6}]decane-3,5-dione, compound (1R,2R,6S,7S)-4-{4-[4-(5-bromopyrimidin-2-yl)piperazin-1-yl]butyl}-(8,9-²H₂)-4-azatricyclo[5.2.1.0^{2,6}]decane-3,5-dione, (1R,2R,6S,7S)-4-{4-[4-((5-²H)pyrimidin-2-yl)piperazin-1-yl]butyl}-(8,9-²H₂⁾⁻4-azatricyclo[5.2.1.0^{2,6}]decane-3,5-dione), (1R,2S,6R,7S)-4-(4-bromobutyl)-4-azatricyclo[5.2.1.0^{2,6}]dec-8-ene-3,5-dione, (1R,2R,6S,7S)-4-{4-[4-(5-bromopyrimidin-2-yl)piperazin-1-yl]-(4-²H₂)butyl}-(8,9-²H₂)-4-azatricyclo[5.2.1.0^{2,6}]decane-3,5-dione, (1R,2R,6S,7S)-4-{4-[4-((5-²H)pyrimidin-2-yl)piperazin-1-yl]butyl}-(8,9-²H₂)-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]dec-8-ene-3,5-dione, (1R,2S,6R,7S)-4-{4-[4-(pyrimidin-2-yl)piperazin-1-yl]-(4-²H₂)but-2-yn-yl}-(8,9-²H₂)-4-azatricyclo[5.2.1.0^{2,6}]decane-3,5-dione, (1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione, (8,9-²H₂)-4-azatricyclo[5.2.1.0^{2,6}]decane-3,5-dione, (1R,2S,6R,7S)-4-(prop-2-yn-1-yl)-4(8,9-²H₂)-4-azatricyclo[5.2.1.0^{2,6}]decane-3,5-dione, (1R,2S,6R,7S)-4-{4-[4-((5-²H)pyrimidin-2-yl)piperazin-1-yl]-(4-²H₂)but-2-yn-1-yl}-4-azatricyclo[5.2.1.0^{2,6}]dec-8-ene-3,5-dione, ((1R,2R,6S,7S)-4-{4-[4-(pyrimidin-2-yl)piperazin-1-yl]-(4-²H₂)butyl}-(8,9-²H₂)-4-azatricyclo[5.2.1.0^{2,6}]decane-3,5-dione, 1R,2S,6R,7S)-4-{4-[4-(5-bromopyrimidin-2-yl)piperazin-1-yl]-(4-²H₂)but-2-yn-1-yl}-4-azatricyclo[5.2.1.0^{2,6}]dec-8-ene-3,5-dione, (1R,2S,6R,7S)-4-{4-[4-((5-²H)pyrimidin-2-yl)piperazin-1-yl]-(4-²H₂)but-2-yn-1-yl}-(8,9-²H₂)-4-azatricyclo[5.2.1.0^{2,6}]decane-3,5-dione and ((1R,2R,6S,7S)-4-{4-[4-((5-²H)pyrimidin-2-yl)piperazin-1-yl]-(4-²H₂)butyl}-(8,9-²H₂)-4-azatricyclo[5.2.1.0^{2,6}]decane-3,5-dione.
